SEQUENCE LISTING (52 sequences)

SEQ ID NO: 1 - DNA, 1664 bp, Homo sapiens
  source 1..1664: mol_type = genomic DNA
cttcttctttttcctacagctcctgggcaacgtgctggttattgtgctgtctcatcattttggcaaagaattcctcgaagatccgaaggggttcaagcttatcatcatgatagtgtttgtcaggttcaactccagccatggtttcccagtggaggtcgattctgacaccagcatcttccagctcaaggaggtggttgctaagcgacagggggttccggctgaccagttgcgtgtgattttcgcagggaaggagctgaggaatgactggactgtgcagaattgtgacctggatcagcagagcattgttcacattgtgcagagaccgtggagaaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgacagcaggaaggactcaccaccagctggaagtccagcaggtagatcaatctacaacagcttttatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacatcagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgttgggaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactggttcgacgtgtaaagtcgacaacttgtttattgcagcttataatggttacaaataaagcaatagcatcacaaatttcacaaataaagcatttttttcactgcattctagttgtggtttgtccaaactcatcaatgtatcttatcatgtctggatcagatctgaggaaccccta

SEQ ID NO: 2 - protein, 465 aa, Homo sapiens
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDV

SEQ ID NO: 3 - DNA, 1664 bp, synthetic construct
  misc_feature 1..1664: note = W183Y PARK2 mutant cDNA
  source 1..1664: mol_type = other DNA
cttcttctttttcctacagctcctgggcaacgtgctggttattgtgctgtctcatcattttggcaaagaattcctcgaagatccgaaggggttcaagcttatcatcatgatagtgtttgtcaggttcaactccagccatggtttcccagtggaggtcgattctgacaccagcatcttccagctcaaggaggtggttgctaagcgacagggggttccggctgaccagttgcgtgtgattttcgcagggaaggagctgaggaatgactggactgtgcagaattgtgacctggatcagcagagcattgttcacattgtgcagagaccgtggagaaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgacagcaggaaggactcaccaccagctggaagtccagcaggtagatcaatctacaacagcttttatgtgtattgcaaaggcccctgtcaaagagtgcagccgggaaaactcagggtacagtgcagcacctgcaggcaggcaacgctcaccttgacccagggtccatcttgctacgatgatgttttaattccaaaccggatgagtggtgaatgccaatccccacactgccctgggactagtgcagaatttttctttaaatgtggagcacaccccacctctgacaaggaaacatcagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgttgggaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactggttcgacgtgtaaagtcgacaacttgtttattgcagcttataatggttacaaataaagcaatagcatcacaaatttcacaaataaagcatttttttcactgcattctagttgtggtttgtccaaactcatcaatgtatcttatcatgtctggatcagatctgaggaaccccta

SEQ ID NO: 4 - protein, 465 aa, synthetic construct
  REGION 1..465: note = W183Y PARK2 mutant polypeptide
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCYDDVLIPNRMSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDV

SEQ ID NO: 5 - DNA, 1664 bp, synthetic construct
  misc_feature 1..1664: note = F208Y PARK2 mutant cDNA
  source 1..1664: mol_type = other DNA
cttcttctttttcctacagctcctgggcaacgtgctggttattgtgctgtctcatcattttggcaaagaattcctcgaagatccgaaggggttcaagcttatcatcatgatagtgtttgtcaggttcaactccagccatggtttcccagtggaggtcgattctgacaccagcatcttccagctcaaggaggtggttgctaagcgacagggggttccggctgaccagttgcgtgtgattttcgcagggaaggagctgaggaatgactggactgtgcagaattgtgacctggatcagcagagcattgttcacattgtgcagagaccgtggagaaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgacagcaggaaggactcaccaccagctggaagtccagcaggtagatcaatctacaacagcttttatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatacttctttaaatgtggagcacaccccacctctgacaaggaaacatcagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgttgggaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactggttcgacgtgtaaagtcgacaacttgtttattgcagcttataatggttacaaataaagcaatagcatcacaaatttcacaaataaagcatttttttcactgcattctagttgtggtttgtccaaactcatcaatgtatcttatcatgtctggatcagatctgaggaaccccta

SEQ ID NO: 6 - protein, 465 aa, synthetic construct
  REGION 1..465: note = F208Y PARK2 mutant polypeptide
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEYFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDV

SEQ ID NO: 7 - DNA, 1664 bp, synthetic construct
  misc_feature 1..1664: note = F463Y PARK2 mutant cDNA
  source 1..1664: mol_type = other DNA
cttcttctttttcctacagctcctgggcaacgtgctggttattgtgctgtctcatcattttggcaaagaattcctcgaagatccgaaggggttcaagcttatcatcatgatagtgtttgtcaggttcaactccagccatggtttcccagtggaggtcgattctgacaccagcatcttccagctcaaggaggtggttgctaagcgacagggggttccggctgaccagttgcgtgtgattttcgcagggaaggagctgaggaatgactggactgtgcagaattgtgacctggatcagcagagcattgttcacattgtgcagagaccgtggagaaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgacagcaggaaggactcaccaccagctggaagtccagcaggtagatcaatctacaacagcttttatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacatcagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgttgggaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactggtacgacgtgtaaagtcgacaacttgtttattgcagcttataatggttacaaataaagcaatagcatcacaaatttcacaaataaagcatttttttcactgcattctagttgtggtttgtccaaactcatcaatgtatcttatcatgtctggatcagatctgaggaaccccta

SEQ ID NO: 8 - protein, 465 aa, synthetic construct
  REGION 1..465: note = F463Y PARK2 mutant polypeptide
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWYDV

SEQ ID NO: 9 - DNA, 4718 bp, Dependoparvovirus Adeno-associated virus - 1
  source 1..4718: mol_type = other DNA
ttgcccactccctctctgcgcgctcgctcgctcggtggggcctgcggaccaaaggtccgcagacggcagagctctgctctgccggccccaccgagcgagcgagcgcgcagagagggagtgggcaactccatcactaggggtaatcgcgaagcgcctcccacgctgccgcgtcagcgctgacgtaaattacgtcataggggagtggtcctgtattagctgtcacgtgagtgcttttgcgacattttgcgacaccacgtggccatttagggtatatatggccgagtgagcgagcaggatctccattttgaccgcgaaatttgaacgagcagcagccatgccgggcttctacgagatcgtgatcaaggtgccgagcgacctggacgagcacctgccgggcatttctgactcgtttgtgagctgggtggccgagaaggaatgggagctgcccccggattctgacatggatctgaatctgattgagcaggcacccctgaccgtggccgagaagctgcagcgcgacttcctggtccaatggcgccgcgtgagtaaggccccggaggccctcttctttgttcagttcgagaagggcgagtcctacttccacctccatattctggtggagaccacgggggtcaaatccatggtgctgggccgcttcctgagtcagattagggacaagctggtgcagaccatctaccgcgggatcgagccgaccctgcccaactggttcgcggtgaccaagacgcgtaatggcgccggaggggggaacaaggtggtggacgagtgctacatccccaactacctcctgcccaagactcagcccgagctgcagtgggcgtggactaacatggaggagtatataagcgcctgtttgaacctggccgagcgcaaacggctcgtggcgcagcacctgacccacgtcagccagacccaggagcagaacaaggagaatctgaaccccaattctgacgcgcctgtcatccggtcaaaaacctccgcgcgctacatggagctggtcgggtggctggtggaccggggcatcacctccgagaagcagtggatccaggaggaccaggcctcgtacatctccttcaacgccgcttccaactcgcggtcccagatcaaggccgctctggacaatgccggcaagatcatggcgctgaccaaatccgcgcccgactacctggtaggccccgctccgcccgcggacattaaaaccaaccgcatctaccgcatcctggagctgaacggctacgaacctgcctacgccggctccgtctttctcggctgggcccagaaaaggttcgggaagcgcaacaccatctggctgtttgggccggccaccacgggcaagaccaacatcgcggaagccatcgcccacgccgtgcccttctacggctgcgtcaactggaccaatgagaactttcccttcaatgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagccgttgcaggaccggatgttcaaatttgaactcacccgccgtctggagcatgactttggcaaggtgacaaagcaggaagtcaaagagttcttccgctgggcgcaggatcacgtgaccgaggtggcgcatgagttctacgtcagaaagggtggagccaacaaaagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaagacatgcgagagaatgaatcagaatttcaacatttgcttcacgcacgggacgagagactgttcagagtgcttccccggcgtgtcagaatctcaaccggtcgtcagaaagaggacgtatcggaaactctgtgccattcatcatctgctggggcgggctcccgagattgcttgctcggcctgcgatctggtcaacgtggacctggatgactgtgtttctgagcaataaatgacttaaaccaggtatggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaattacgtgttaatcaataaaccggttgattcgtttcagttgaactttggtctcctgtccttcttatcttatcggttaccatggttatagcttacacattaactgcttggttgcgcttcgcgataaaagacttacgtcatcgggttacccctagtgatggagttgcccactccctctctgcgcgctcgctcgctcggtggggcctgcggaccaaaggtccgcagacggcagagctctgctctgccggccccaccgagcgagcgagcgcgcagagagggagtgggcaa

SEQ ID NO: 10 - DNA, 4679 bp, Dependoparvovirus Adeno-associated virus - 2
  source 1..4679: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 11 - DNA, 4722 bp, Dependoparvovirus Adeno-associated virus - 3
  source 1..4722: mol_type = other DNA
tggccactccctctatgcgcactcgctcgctcggtggggcctggcgaccaaaggtcgccagacggacgtgctttgcacgtccggccccaccgagcgagcgagtgcgcatagagggagtggccaactccatcactagaggtatggcagtgacgtaacgcgaagcgcgcgaagcgagaccacgcctaccagctgcgtcagcagtcaggtgacccttttgcgacagtttgcgacaccacgtggccgctgagggtatatattctcgagtgagcgaaccaggagctccattttgaccgcgaaatttgaacgagcagcagccatgccggggttctacgagattgtcctgaaggtcccgagtgacctggacgagcacctgccgggcatttctaactcgtttgttaactgggtggccgagaaggaatgggagctgccgccggattctgacatggatccgaatctgattgagcaggcacccctgaccgtggccgaaaagcttcagcgcgagttcctggtggagtggcgccgcgtgagtaaggccccggaggccctcttttttgtccagttcgaaaagggggagacctacttccacctgcacgtgctgattgagaccatcggggtcaaatccatggtggtcggccgctacgtgagccagattaaagagaagctggtgacccgcatctaccgcggggtcgagccgcagcttccgaactggttcgcggtgaccaaaacgcgaaatggcgccgggggcgggaacaaggtggtggacgactgctacatccccaactacctgctccccaagacccagcccgagctccagtgggcgtggactaacatggaccagtatttaagcgcctgtttgaatctcgcggagcgtaaacggctggtggcgcagcatctgacgcacgtgtcgcagacgcaggagcagaacaaagagaatcagaaccccaattctgacgcgccggtcatcaggtcaaaaacctcagccaggtacatggagctggtcgggtggctggtggaccgcgggatcacgtcagaaaagcaatggattcaggaggaccaggcctcgtacatctccttcaacgccgcctccaactcgcggtcccagatcaaggccgcgctggacaatgcctccaagatcatgagcctgacaaagacggctccggactacctggtgggcagcaacccgccggaggacattaccaaaaatcggatctaccaaatcctggagctgaacgggtacgatccgcagtacgcggcctccgtcttcctgggctgggcgcaaaagaagttcgggaagaggaacaccatctggctctttgggccggccacgacgggtaaaaccaacatcgcggaagccatcgcccacgccgtgcccttctacggctgcgtaaactggaccaatgagaactttcccttcaacgattgcgtcgacaagatggtgatctggtgggaggagggcaagatgacggccaaggtcgtggagagcgccaaggccattctgggcggaagcaaggtgcgcgtggaccaaaagtgcaagtcatcggcccagatcgaacccactcccgtgatcgtcacctccaacaccaacatgtgcgccgtgattgacgggaacagcaccaccttcgagcatcagcagccgctgcaggaccggatgtttaaatttgaacttacccgccgtttggaccatgactttgggaaggtcaccaaacaggaagtaaaggactttttccggtgggcttccgatcacgtgactgacgtggctcatgagttctacgtcagaaagggtggagctaagaaacgccccgcctccaatgacgcggatgtaagcgagccaaaacggcagtgcacgtcacttgcgcagccgacaacgtcagacgcggaagcaccggcggactacgcggacaggtaccaaaacaaatgttctcgtcacgtgggcatgaatctgatgctttttccctgtaaaacatgcgagagaatgaatcaaatttccaatgtctgttttacgcatggtcaaagagactgtggggaatgcttccctggaatgtcagaatctcaacccgtttctgtcgtcaaaaagaagacttatcagaaactgtgtccaattcatcatatcctgggaagggcacccgagattgcctgttcggcctgcgatttggccaatgtggacttggatgactgtgtttctgagcaataaatgacttaaaccaggtatggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtaatcctggttaatcaataaaccgtttaattcgtttcagttgaactttggctcttgtgcacttcttatcttatcttgtttccatggctactgcgtagataagcagcggcctgcggcgcttgcgcttcgcggtttacaactgctggttaatatttaactctcgccatacctctagtgatggagttggccactccctctatgcgcactcgctcgctcggtggggccggacgtgcaaagcacgtccgtctggcgacctttggtcgccaggccccaccgagcgagcgagtgcgcatagagggagtggccaa

SEQ ID NO: 12 - DNA, 4767 bp, Dependoparvovirus Adeno-associated virus - 4
  source 1..4767: mol_type = other DNA
ttggccactccctctatgcgcgctcgctcactcactcggccctggagaccaaaggtctccagactgccggcctctggccggcagggccgagtgagtgagcgagcgcgcatagagggagtggccaactccatcatctaggtttgcccactgacgtcaatgtgacgtcctagggttagggaggtccctgtattagcagtcacgtgagtgtcgtatttcgcggagcgtagcggagcgcataccaagctgccacgtcacagccacgtggtccgtttgcgacagtttgcgacaccatgtggtcaggagggtatataaccgcgagtgagccagcgaggagctccattttgcccgcgaattttgaacgagcagcagccatgccggggttctacgagatcgtgctgaaggtgcccagcgacctggacgagcacctgcccggcatttctgactcttttgtgagctgggtggccgagaaggaatgggagctgccgccggattctgacatggacttgaatctgattgagcaggcacccctgaccgtggccgaaaagctgcaacgcgagttcctggtcgagtggcgccgcgtgagtaaggccccggaggccctcttctttgtccagttcgagaagggggacagctacttccacctgcacatcctggtggagaccgtgggcgtcaaatccatggtggtgggccgctacgtgagccagattaaagagaagctggtgacccgcatctaccgcggggtcgagccgcagcttccgaactggttcgcggtgaccaagacgcgtaatggcgccggaggcgggaacaaggtggtggacgactgctacatccccaactacctgctccccaagacccagcccgagctccagtgggcgtggactaacatggaccagtatataagcgcctgtttgaatctcgcggagcgtaaacggctggtggcgcagcatctgacgcacgtgtcgcagacgcaggagcagaacaaggaaaaccagaaccccaattctgacgcgccggtcatcaggtcaaaaacctccgccaggtacatggagctggtcgggtggctggtggaccgcgggatcacgtcagaaaagcaatggatccaggaggaccaggcgtcctacatctccttcaacgccgcctccaactcgcggtcacaaatcaaggccgcgctggacaatgcctccaaaatcatgagcctgacaaagacggctccggactacctggtgggccagaacccgccggaggacatttccagcaaccgcatctaccgaatcctcgagatgaacgggtacgatccgcagtacgcggcctccgtcttcctgggctgggcgcaaaagaagttcgggaagaggaacaccatctggctctttgggccggccacgacgggtaaaaccaacatcgcggaagccatcgcccacgccgtgcccttctacggctgcgtgaactggaccaatgagaactttccgttcaacgattgcgtcgacaagatggtgatctggtgggaggagggcaagatgacggccaaggtcgtagagagcgccaaggccatcctgggcggaagcaaggtgcgcgtggaccaaaagtgcaagtcatcggcccagatcgacccaactcccgtgatcgtcacctccaacaccaacatgtgcgcggtcatcgacggaaactcgaccaccttcgagcaccaacaaccactccaggaccggatgttcaagttcgagctcaccaagcgcctggagcacgactttggcaaggtcaccaagcaggaagtcaaagactttttccggtgggcgtcagatcacgtgaccgaggtgactcacgagttttacgtcagaaagggtggagctagaaagaggcccgcccccaatgacgcagatataagtgagcccaagcgggcctgtccgtcagttgcgcagccatcgacgtcagacgcggaagctccggtggactacgcggacaggtaccaaaacaaatgttctcgtcacgtgggtatgaatctgatgctttttccctgccggcaatgcgagagaatgaatcagaatgtggacatttgcttcacgcacggggtcatggactgtgccgagtgcttccccgtgtcagaatctcaacccgtgtctgtcgtcagaaagcggacgtatcagaaactgtgtccgattcatcacatcatggggagggcgcccgaggtggcctgctcggcctgcgaactggccaatgtggacttggatgactgtgacatggaacaataaatgactcaaaccagatatgactgacggttaccttccagattggctagaggacaacctctctgaaggcgttcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgcggagttccagcagcggcttcagggcgacacatcgtttgggggcaacctcggcagagcagtcttccaggccaaaaagagggttcttgaacctcttggtctggttgagcaagcgggtgagacggctcctggaaagaagagaccgttgattgaatccccccagcagcccgactcctccacgggtatcggcaaaaaaggcaagcagccggctaaaaagaagctcgttttcgaagacgaaactggagcaggcgacggaccccctgagggatcaacttccggagccatgtctgatgacagtgagatgcgtgcagcagctggcggagctgcagtcgagggcggacaaggtgccgatggagtgggtaatgcctcgggtgattggcattgcgattccacctggtctgagggccacgtcacgaccaccagcaccagaacctgggtcttgcccacctacaacaaccacctctacaagcgactcggagagagcctgcagtccaacacctacaacggattctccaccccctggggatactttgacttcaaccgcttccactgccacttctcaccacgtgactggcagcgactcatcaacaacaactggggcatgcgacccaaagccatgcgggtcaaaatcttcaacatccaggtcaaggaggtcacgacgtcgaacggcgagacaacggtggctaataaccttaccagcacggttcagatctttgcggactcgtcgtacgaactgccgtacgtgatggatgcgggtcaagagggcagcctgcctccttttcccaacgacgtctttatggtgccccagtacggctactgtggactggtgaccggcaacacttcgcagcaacagactgacagaaatgccttctactgcctggagtactttccttcgcagatgctgcggactggcaacaactttgaaattacgtacagttttgagaaggtgcctttccactcgatgtacgcgcacagccagagcctggaccggctgatgaaccctctcatcgaccagtacctgtggggactgcaatcgaccaccaccggaaccaccctgaatgccgggactgccaccaccaactttaccaagctgcggcctaccaacttttccaactttaaaaagaactggctgcccgggccttcaatcaagcagcagggcttctcaaagactgccaatcaaaactacaagatccctgccaccgggtcagacagtctcatcaaatacgagacgcacagcactctggacggaagatggagtgccctgacccccggacctccaatggccacggctggacctgcggacagcaagttcagcaacagccagctcatctttgcggggcctaaacagaacggcaacacggccaccgtacccgggactctgatcttcacctctgaggaggagctggcagccaccaacgccaccgatacggacatgtggggcaacctacctggcggtgaccagagcaacagcaacctgccgaccgtggacagactgacagccttgggagccgtgcctggaatggtctggcaaaacagagacatttactaccagggtcccatttgggccaagattcctcataccgatggacactttcacccctcaccgctgattggtgggtttgggctgaaacacccgcctcctcaaatttttatcaagaacaccccggtacctgcgaatcctgcaacgaccttcagctctactccggtaaactccttcattactcagtacagcactggccaggtgtcggtgcagattgactgggagatccagaaggagcggtccaaacgctggaaccccgaggtccagtttacctccaactacggacagcaaaactctctgttgtgggctcccgatgcggctgggaaatacactgagcctagggctatcggtacccgctacctcacccaccacctgtaataacctgttaatcaataaaccggtttattcgtttcagttgaactttggtctccgtgtccttcttatcttatctcgtttccatggctactgcgtacataagcagcggcctgcggcgcttgcgcttcgcggtttacaactgccggttaatcagtaacttctggcaaaccagatgatggagttggccacattagctatgcgcgctcgctcactcactcggccctggagaccaaaggtctccagactgccggcctctggccggcagggccgagtgagtgagcgagcgcgcatagagggagtggccaa

SEQ ID NO: 13 - DNA, 4642 bp, Dependoparvovirus Adeno-associated virus - 5
  source 1..4642: mol_type = other DNA
ctctcccccctgtcgcgttcgctcgctcgctggctcgtttgggggggtggcagctcaaagagctgccagacgacggccctctggccgtcgcccccccaaacgagccagcgagcgagcgaacgcgacaggggggagagtgccacactctcaagcaagggggttttgtaagcagtgatgtcataatgatgtaatgcttattgtcacgcgatagttaatgattaacagtcatgtgatgtgttttatccaataggaagaaagcgcgcgtatgagttctcgcgagacttccggggtataaaagaccgagtgaacgagcccgccgccattctttgctctggactgctagaggaccctcgctgccatggctaccttctatgaagtcattgttcgcgtcccatttgacgtggaggaacatctgcctggaatttctgacagctttgtggactgggtaactggtcaaatttgggagctgcctccagagtcagatttaaatttgactctggttgaacagcctcagttgacggtggctgatagaattcgccgcgtgttcctgtacgagtggaacaaattttccaagcaggagtccaaattctttgtgcagtttgaaaagggatctgaatattttcatctgcacacgcttgtggagacctccggcatctcttccatggtcctcggccgctacgtgagtcagattcgcgcccagctggtgaaagtggtcttccagggaattgaaccccagatcaacgactgggtcgccatcaccaaggtaaagaagggcggagccaataaggtggtggattctgggtatattcccgcctacctgctgccgaaggtccaaccggagcttcagtgggcgtggacaaacctggacgagtataaattggccgccctgaatctggaggagcgcaaacggctcgtcgcgcagtttctggcagaatcctcgcagcgctcgcaggaggcggcttcgcagcgtgagttctcggctgacccggtcatcaaaagcaagacttcccagaaatacatggcgctcgtcaactggctcgtggagcacggcatcacttccgagaagcagtggatccaggaaaatcaggagagctacctctccttcaactccaccggcaactctcggagccagatcaaggccgcgctcgacaacgcgaccaaaattatgagtctgacaaaaagcgcggtggactacctcgtggggagctccgttcccgaggacatttcaaaaaacagaatctggcaaatttttgagatgaatggctacgacccggcctacgcgggatccatcctctacggctggtgtcagcgctccttcaacaagaggaacaccgtctggctctacggacccgccacgaccggcaagaccaacatcgcggaggccatcgcccacactgtgcccttttacggctgcgtgaactggaccaatgaaaactttccctttaatgactgtgtggacaaaatgctcatttggtgggaggagggaaagatgaccaacaaggtggttgaatccgccaaggccatcctggggggctcaaaggtgcgggtcgatcagaaatgtaaatcctctgttcaaattgattctacccctgtcattgtaacttccaatacaaacatgtgtgtggtggtggatgggaattccacgacctttgaacaccagcagccgctggaggaccgcatgttcaaatttgaactgactaagcggctcccgccagattttggcaagattactaagcaggaagtcaaggacttttttgcttgggcaaaggtcaatcaggtgccggtgactcacgagtttaaagttcccagggaattggcgggaactaaaggggcggagaaatctctaaaacgcccactgggtgacgtcaccaatactagctataaaagtctggagaagcgggccaggctctcatttgttcccgagacgcctcgcagttcagacgtgactgttgatcccgctcctctgcgaccgctcaattggaattcaaggtatgattgcaaatgtgactatcatgctcaatttgacaacatttctaacaaatgtgatgaatgtgaatatttgaatcggggcaaaaatggatgtatctgtcacaatgtaactcactgtcaaatttgtcatgggattcccccctgggaaaaggaaaacttgtcagattttggggattttgacgatgccaataaagaacagtaaataaagcgagtagtcatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaacccattcatgtcgcataccctcaataaaccgtgtattcgtgtcagtaaaatactgcctcttgtggtcattcaatgaataacagcttacaacatctacaaaacctccttgcttgagagtgtggcactctcccccctgtcgcgttcgctcgctcgctggctcgtttgggggggtggcagctcaaagagctgccagacgacggccctctggccgtcgcccccccaaacgagccagcgagcgagcgaacgcgacaggggggagag

SEQ ID NO: 14 - DNA, 4683 bp, Dependoparvovirus Adeno-associated virus - 6
  source 1..4683: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagcgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgacttcctggtccagtggcgccgcgtgagtaaggccccggaggccctcttctttgttcagttcgagaagggcgagtcctacttccacctccatattctggtggagaccacgggggtcaaatccatggtgctgggccgcttcctgagtcagattagggacaagctggtgcagaccatctaccgcgggatcgagccgaccctgcccaactggttcgcggtgaccaagacgcgtaatggcgccggaggggggaacaaggtggtggacgagtgctacatccccaactacctcctgcccaagactcagcccgagctgcagtgggcgtggactaacatggaggagtatataagcgcgtgtttaaacctggccgagcgcaaacggctcgtggcgcacgacctgacccacgtcagccagacccaggagcagaacaaggagaatctgaaccccaattctgacgcgcctgtcatccggtcaaaaacctccgcacgctacatggagctggtcgggtggctggtggaccggggcatcacctccgagaagcagtggatccaggaggaccaggcctcgtacatctccttcaacgccgcctccaactcgcggtcccagatcaaggccgctctggacaatgccggcaagatcatggcgctgaccaaatccgcgcccgactacctggtaggccccgctccgcccgccgacattaaaaccaaccgcatttaccgcatcctggagctgaacggctacgaccctgcctacgccggctccgtctttctcggctgggcccagaaaaggttcggaaaacgcaacaccatctggctgtttgggccggccaccacgggcaagaccaacatcgcggaagccatcgcccacgccgtgcccttctacggctgcgtcaactggaccaatgagaactttcccttcaacgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgatcccacccccgtgatcgtcacctccaacaccaacatgtgcgccgtgattgacgggaacagcaccaccttcgagcaccagcagccgttgcaggaccggatgttcaaatttgaactcacccgccgtctggagcatgactttggcaaggtgacaaagcaggaagtcaaagagttcttccgctgggcgcaggatcacgtgaccgaggtggcgcatgagttctacgtcagaaagggtggagccaacaagagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaaaacatgcgagagaatgaatcagaatttcaacatttgcttcacgcacgggaccagagactgttcagaatgtttccccggcgtgtcagaatctcaaccggtcgtcagaaagaggacgtatcggaaactctgtgccattcatcatctgctggggcgggctcccgagattgcttgctcggcctgcgatctggtcaacgtggatctggatgactgtgtttctgagcaataaatgacttaaaccaggtatggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaattgtgtgttaatcaataaaccggttaattcgtgtcagttgaactttggtctcatgtcgttattatcttatctggtcaccatagcaaccggttacacattaactgcttagttgcgcttcgcgaatacccctagtgatggagttgcccactccctctatgcgcgctcgctcgctcggtggggccggcagagcagagctctgccgtctgcggacctttggtccgcaggccccaccgagcgagcgagcgcgcatagagggagtgggcaa

SEQ ID NO: 15 - DNA, 4721 bp, Dependoparvovirus Adeno-associated virus - 7
  source 1..4721: mol_type = other DNA
ttggccactccctctatgcgcgctcgctcgctcggtggggcctgcggaccaaaggtccgcagacggcagagctctgctctgccggccccaccgagcgagcgagcgcgcatagagggagtggccaactccatcactaggggtaccgcgaagcgcctcccacgctgccgcgtcagcgctgacgtaaatcacgtcataggggagtggtcctgtattagctgtcacgtgagtgcttttgcgacattttgcgacaccacgtggccatttgaggtatatatggccgagtgagcgagcaggatctccattttgaccgcgaaatttgaacgagcagcagccatgccgggtttctacgagatcgtgatcaaggtgccgagcgacctggacgagcacctgccgggcatttctgactcgtttgtgaactgggtggccgagaaggaatgggagctgcccccggattctgacatggatctgaatctgatcgagcaggcacccctgaccgtggccgagaagctgcagcgcgacttcctggtccaatggcgccgcgtgagtaaggccccggaggccctgttctttgttcagttcgagaagggcgagagctacttccaccttcacgttctggtggagaccacgggggtcaagtccatggtgctaggccgcttcctgagtcagattcgggagaagctggtccagaccatctaccgcggggtcgagcccacgctgcccaactggttcgcggtgaccaagacgcgtaatggcgccggcggggggaacaaggtggtggacgagtgctacatccccaactacctcctgcccaagacccagcccgagctgcagtgggcgtggactaacatggaggagtatataagcgcgtgtttgaacctggccgaacgcaaacggctcgtggcgcagcacctgacccacgtcagccagacgcaggagcagaacaaggagaatctgaaccccaattctgacgcgcccgtgatcaggtcaaaaacctccgcgcgctacatggagctggtcgggtggctggtggaccggggcatcacctccgagaagcagtggatccaggaggaccaggcctcgtacatctccttcaacgccgcctccaactcgcggtcccagatcaaggccgcgctggacaatgccggcaagatcatggcgctgaccaaatccgcgcccgactacctggtggggccctcgctgcccgcggacattaaaaccaaccgcatctaccgcatcctggagctgaacgggtacgatcctgcctacgccggctccgtctttctcggctgggcccagaaaaagttcgggaagcgcaacaccatctggctgtttgggcccgccaccaccggcaagaccaacattgcggaagccatcgcccacgccgtgcccttctacggctgcgtcaactggaccaatgagaactttcccttcaacgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagccgttgcaggaccggatgttcaaatttgaactcacccgccgtctggagcacgactttggcaaggtgacgaagcaggaagtcaaagagttcttccgctgggccagtgatcacgtgaccgaggtggcgcatgagttctacgtcagaaagggcggagccagcaaaagacccgcccccgatgacgcggatataagcgagcccaagcgggcctgcccctcagtcgcggatccatcgacgtcagacgcggaaggagctccggtggactttgccgacaggtaccaaaacaaatgttctcgtcacgcgggcatgattcagatgctgtttccctgcaaaacgtgcgagagaatgaatcagaatttcaacatttgcttcacacacggggtcagagactgtttagagtgtttccccggcgtgtcagaatctcaaccggtcgtcagaaaaaagacgtatcggaaactctgcgcgattcatcatctgctggggcgggcgcccgagattgcttgctcggcctgcgacctggtcaacgtggacctggacgactgcgtttctgagcaataaatgacttaaaccaggtatggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctgcaaagaagagaccggtagagccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagtgaaactgcaggtagtaccaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattcctcagtacggctacctgactctcaacaatggcagtcagtctgtgggacgttcctccttctactgcctggagtacttcccctctcagatgctgagaacgggcaacaactttgagttcagctacagcttcgaggacgtgcctttccacagcagctacgcacacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacctccaactttgaaaagcagactggtgtggactttgccgttgacagccagggtgtttactctgagcctcgccctattggcactcgttacctcacccgtaatctgtaattgcatgttaatcaataaaccggttgattcgtttcagttgaactttggtctcctgtgcttcttatcttatcggtttccatagcaactggttacacattaactgcttgggtgcgcttcacgataagaacactgacgtcaccgcggtacccctagtgatggagttggccactccctctatgcgcgctcgctcgctcggtggggcctgcggaccaaaggtccgcagacggcagagctctgctctgccggccccaccgagcgagcgagcgcgcatagagggagtggccaa

SEQ ID NO: 16 - DNA, 4393 bp, Dependoparvovirus Adeno-associated virus - 8
  source 1..4393: mol_type = other DNA
cagagagggagtggccaactccatcactaggggtagcgcgaagcgcctcccacgctgccgcgtcagcgctgacgtaaattacgtcataggggagtggtcctgtattagctgtcacgtgagtgcttttgcggcattttgcgacaccacgtggccatttgaggtatatatggccgagtgagcgagcaggatctccattttgaccgcgaaatttgaacgagcagcagccatgccgggcttctacgagatcgtgatcaaggtgccgagcgacctggacgagcacctgccgggcatttctgactcgtttgtgaactgggtggccgagaaggaatgggagctgcccccggattctgacatggatcggaatctgatcgagcaggcacccctgaccgtggccgagaagctgcagcgcgacttcctggtccaatggcgccgcgtgagtaaggccccggaggccctcttctttgttcagttcgagaagggcgagagctactttcacctgcacgttctggtcgagaccacgggggtcaagtccatggtgctaggccgcttcctgagtcagattcgggaaaagcttggtccagaccatctacccgcggggtcgagccccaccttgcccaactggttcgcggtgaccaaagacgcggtaatggcgccggcgggggggaacaaggtggtggacgagtgctacatccccaactacctcctgcccaagactcagcccgagctgcagtgggcgtggactaacatggaggagtatataagcgcgtgcttgaacctggccgagcgcaaacggctcgtggcgcagcacctgacccacgtcagccagacgcaggagcagaacaaggagaatctgaaccccaattctgacgcgcccgtgatcaggtcaaaaacctccgcgcgctatatggagctggtcgggtggctggtggaccggggcatcacctccgagaagcagtggatccaggaggaccaggcctcgtacatctccttcaacgccgcctccaactcgcggtcccagatcaaggccgcgctggacaatgccggcaagatcatggcgctgaccaaatccgcgcccgactacctggtggggccctcgctgcccgcggacattacccagaaccgcatctaccgcatcctcgctctcaacggctacgaccctgcctacgccggctccgtctttctcggctgggctcagaaaaagttcgggaaacgcaacaccatctggctgtttggacccgccaccaccggcaagaccaacattgcggaagccatcgcccacgccgtgcccttctacggctgcgtcaactggaccaatgagaactttcccttcaatgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagcctctccaggaccggatgtttaagttcgaactcacccgccgtctggagcacgactttggcaaggtgacaaagcaggaagtcaaagagttcttccgctgggccagtgatcacgtgaccgaggtggcgcatgagttttacgtcagaaagggcggagccagcaaaagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaaaacgtgcgagagaatgaatcagaatttcaacatttgcttcacacacggggtcagagactgctcagagtgtttccccggcgtgtcagaatctcaaccggtcgtcagaaagaggacgtatcggaaactctgtgcgattcatcatctgctggggcgggctcccgagattgcttgctcggcctgcgatctggtcaacgtggacctggatgactgtgtttctgagcaataaatgacttaaaccaggtatggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaattgcctgttaatcaataaaccggttgattcgtttcagttgaactttggtctctgcg

SEQ ID NO: 17 - DNA, 2211 bp, Dependoparvovirus Adeno-associated virus - 9
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 18 - DNA, 4102 bp, Dependoparvovirus Adeno-associated virus - 10
  source 1..4102: mol_type = other DNA
atgccgggcttctacgagatcgtgatcaaggtgccgagcgacctggacgagcacctgccgggcatttctgactcgtttgtgaactgggtggccgagaaggaatgggagctgcccccggattctgacatggatcggaatctgatcgagcaggcacccctgaccgtggccgagaagctgcagcgcgacttcctggtccactggcgccgcgtgagtaaggccccggaggccctcttctttgttcagttcgagaagggcgagtcctactttcacctgcacgttctggtcgagaccacgggggtcaagtccatggtcctgggccgcttcctgagtcagatcagagacaggctggtgcagaccatctaccgcggggtagagcccacgctgcccaactggttcgcggtgaccaagacgcgaaatggcgccggcggggggaacaaggtggtggacgagtgctacatccccaactacctcctgcccaagacgcagcccgagctgcagtgggcgtggactaacatggaggagtatataagcgcgtgtctgaacctcgcggagcgtaaacggctcgtggcgcagcacctgacccacgtcagccagacgcaggagcagaacaaggagaatctgaacccgaattctgacgcgcccgtgatcaggtcaaaaacctccgcgcgctacatggagctggtcgggtggctggtggaccggggcatcacctccgagaagcagtggatccaggaggaccaggcctcgtacatctccttcaacgccgcctccaactcgcggtcccagatcaaggccgcgctggacaatgccggaaagatcatggcgctgaccaaatccgcgcccgactacctggtaggcccgtccttacccgcggacattaaggccaaccgcatctaccgcatcctggagctcaacggctacgaccccgcctacgccggctccgtcttcctgggctgggcgcagaaaaagttcggtaaaaggaatacaatttggctgttcgggcccgccaccaccggcaagaccaacatcgcggaagccatcgcccacgccgtgcccttctacggctgcgtcaactggaccaatgagaactttcccttcaacgattgcgtcgacaagatggtgatctggtgggaggagggcaagatgaccgccaaggtcgtggagtccgccaaggccattctgggcggaagcaaggtgcgcgtcgaccaaaagtgcaagtcctcggcccagatcgaccccacgcccgtgatcgtcacctccaacaccaacatgtgcgccgtgatcgacgggaacagcaccaccttcgagcaccagcagcccctgcaggaccgcatgttcaagttcgagctcacccgccgtctggagcacgactttggcaaggtgaccaagcaggaagtcaaagagttcttccgctgggctcaggatcacgtgactgaggtgacgcatgagttctacgtcagaaagggcggagccaccaaaagacccgcccccagtgacgcggatataagcgagcccaagcgggcctgcccctcagttgcggagccatcgacgtcagacgcggaagcaccggtggactttgcggacaggtaccaaaacaaatgttctcgtcacgcgggcatgcttcagatgctgtttccctgcaagacatgcgagagaatgaatcagaatttcaacgtctgcttcacgcacggggtcagagactgctcagagtgcttccccggcgcgtcagaatctcaacctgtcgtcagaaaaaagacgtatcagaaactgtgcgcgattcatcatctgctggggcgggcacccgagattgcgtgttcggcctgcgatctcgtcaacgtggacttggatgactgtgtttctgagcaataaatgacttaaaccaggtatggctgctgacggttatcttccagattggctcgaggacaacctctctgagggcattcgcgagtggtgggacctgaaacctggagcccccaagcccaaggccaaccagcagaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaagctgctaagacggctcctggaaagaagagaccggtagaaccgtcacctcagcgttcccccgactcctccacgggcatcggcaagaaaggccagcagcccgctaaaaagagactgaactttgggcagactggcgagtcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctgcccacctacaacaaccacctctacaagcaaatctccaacgggacatcgggaggaagcaccaacgacaacacctacttcggctacagcaccccctgggggtattttgacttcaacagattccactgccacttctcaccacgtgactggcagcgactcatcaacaacaactggggattccggccaaaaagactcagcttcaagctcttcaacatccaggtcaaggaggtcacgcagaatgaaggcaccaagaccatcgccaataaccttaccagcacgattcaggtatttacggactcggaataccagctgccgtacgtcctcggctccgcgcaccagggctgcctgcctccgttcccggcggatgtcttcatgattccccagtacggctacctgacactgaacaatggaagtcaagccgtaggccgttcctccttctactgcctggaatattttccatctcaaatgctgcgaactggaaacaattttgaattcagctacaccttcgaggacgtgcctttccacagcagctacgcacacagccagagcttggaccgactgatgaatcctctcattgaccagtacctgtactacttatccagaactcagtccacaggaggaactcaaggtacccagcaattgttattttctcaagctgggcctgcaaacatgtcggctcaggccaagaactggctgcctggaccttgctaccggcagcagcgagtctccacgacactgtcgcaaaacaacaacagcaactttgcttggactggtgccaccaaatatcacctgaacggaagagactctctggtgaatcccggtgtcgccatggcaacccacaaggacgacgaggaacgcttcttcccgtcgagcggagtcctgatgtttggaaaacagggtgctggaagagacaatgtggactacagcagcgttatgctaacaagcgaagaagaaattaaaaccactaaccctgtagccacagaacaatacggcgtggtggctgacaacttgcagcaagccaatacagggcctattgtgggaaatgtcaacagccaaggagccttacctggcatggtctggcagaaccgagacgtgtacctgcagggtcccatctgggccaagattcctcacacggacggcaactttcacccgtctcctctgatgggcggctttggacttaaacacccgcctccacagatcctgatcaagaacacgccggtacctgcggatcctccaacaacgttcagccaggcgaaattggcttccttcatcacgcagtacagcaccggacaggtcagcgtggaaatcgagtgggagctgcagaaggagaacagcaaacgctggaacccagagattcagtacacttcaaactactacaaatctacaaatgtggactttgctgtcaatacagagggaacttattctgagcctcgccccattggtactcgttatctgacacgtaatctgtaa

SEQ ID NO: 19 - DNA, 4087 bp, Dependoparvovirus Adeno-associated virus - 11
  source 1..4087: mol_type = other DNA
atgccgggcttctacgagatcgtgatcaaggtgccgagcgacctggacgagcacctgccgggcatttctgactcgtttgtgaactgggtggccgagaaggaatgggagctgcccccggattctgacatggatcggaatctgatcgagcaggcacccctgaccgtggccgagaagctgcagcgcgacttcctggtccactggcgccgcgtgagtaaggccccggaggccctcttctttgttcagttcgagaagggcgagtcctacttccacctccacgttctcgtcgagaccacgggggtcaagtccatggtcctgggccgcttcctgagtcagatcagagacaggctggtgcagaccatctaccgcggggtcgagcccacgctgcccaactggttcgcggtgaccaagacgcgaaatggcgccggcggggggaacaaggtggtggacgagtgctacatccccaactacctcctgcccaagacccagcccgagctgcagtgggcgtggactaacatggaggagtatataagcgcgtgtctaaacctcgcggagcgtaaacggctcgtggcgcagcacctgacccacgtcagccagacgcaggagcagaacaaggagaatctgaacccgaattctgacgcgcccgtgatcaggtcaaaaacctccgcgcgctacatggagctggtcgggtggctggtggaccggggcatcacctccgagaagcagtggatccaggaggaccaggcctcgtacatctccttcaacgccgcctccaactcgcggtcccagatcaaggccgcgctggacaatgccggaaagatcatggcgctgaccaaatccgcgcccgactacctggtaggcccgtccttacccgcggacattaaggccaaccgcatctaccgcatcctggagctcaacggctacgaccccgcctacgccggctccgtcttcctgggctgggcgcagaaaaagttcggtaaacgcaacaccatctggctgtttgggcccgccaccaccggcaagaccaacatcgcggaagccatagcccacgccgtgcccttctacggctgcgtgaactggaccaatgagaactttcccttcaacgattgcgtcgacaagatggtgatctggtgggaggagggcaagatgaccgccaaggtcgtggagtccgccaaggccattctgggcggaagcaaggtgcgcgtggaccaaaagtgcaagtcctcggcccagatcgaccccacgcccgtgatcgtcacctccaacaccaacatgtgcgccgtgatcgacgggaacagcaccaccttcgagcaccagcagccgctgcaggaccgcatgttcaagttcgagctcacccgccgtctggagcacgactttggcaaggtgaccaagcaggaagtcaaagagttcttccgctgggctcaggatcacgtgactgaggtggcgcatgagttctacgtcagaaagggcggagccaccaaaagacccgcccccagtgacgcggatataagcgagcccaagcgggcctgcccctcagttccggagccatcgacgtcagacgcggaagcaccggtggactttgcggacaggtaccaaaacaaatgttctcgtcacgcgggcatgcttcagatgctgtttccctgcaagacatgcgagagaatgaatcagaatttcaacgtctgcttcacgcacggggtcagagactgctcagagtgcttccccggcgcgtcagaatctcaacccgtcgtcagaaaaaagacgtatcagaaactgtgcgcgattcatcatctgctggggcgggcacccgagattgcgtgttcggcctgcgatctcgtcaacgtggacttggatgactgtgtttctgagcaataaatgacttaaaccaggtatggctgctgacggttatcttccagattggctcgaggacaacctctctgagggcattcgcgagtggtgggacctgaaacctggagccccgaagcccaaggccaaccagcagaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggtactcgaacctctgggcctggttgaagaaggtgctaaaacggctcctggaaagaagagaccgttagagtcaccacaagagcccgactcctcctcgggcatcggcaaaaaaggcaaacaaccagccagaaagaggctcaactttgaagaggacactggagccggagacggaccccctgaaggatcagataccagcgccatgtcttcagacattgaaatgcgtgcagcaccgggcggaaatgctgtcgatgcgggacaaggttccgatggagtgggtaatgcctcgggtgattggcattgcgattccacctggtctgagggcaaggtcacaacaacctcgaccagaacctgggtcttgcccacctacaacaaccacttgtacctgcgtctcggaacaacatcaagcagcaacacctacaacggattctccaccccctggggatattttgacttcaacagattccactgtcacttctcaccacgtgactggcaaagactcatcaacaacaactggggactacgaccaaaagccatgcgcgttaaaatcttcaatatccaagttaaggaggtcacaacgtcgaacggcgagactacggtcgctaataaccttaccagcacggttcagatatttgcggactcgtcgtatgagctcccgtacgtgatggacgctggacaagaggggagcctgcctcctttccccaatgacgtgttcatggtgcctcaatatggctactgtggcatcgtgactggcgagaatcagaaccaaacggacagaaacgctttctactgcctggagtattttccttcgcaaatgttgagaactggcaacaactttgaaatggcttacaactttgagaaggtgccgttccactcaatgtatgctcacagccagagcctggacagactgatgaatcccctcctggaccagtacctgtggcacttacagtcgactacctctggagagactctgaatcaaggcaatgcagcaaccacatttggaaaaatcaggagtggagactttgccttttacagaaagaactggctgcctgggccttgtgttaaacagcagagattctcaaaaactgccagtcaaaattacaagattcctgccagcgggggcaacgctctgttaaagtatgacacccactataccttaaacaaccgctggagcaacatcgcgcccggacctccaatggccacagccggaccttcggatggggacttcagtaacgcccagcttatattccctggaccatctgttaccggaaatacaacaacttcagccaacaatctgttgtttacatcagaagaagaaattgctgccaccaacccaagagacacggacatgtttggccagattgctgacaataatcagaatgctacaactgctcccataaccggcaacgtgactgctatgggagtgctgcctggcatggtgtggcaaaacagagacatttactaccaagggccaatttgggccaagatcccacacgcggacggacattttcatccttcaccgctgattggtgggtttggactgaaacacccgcctccccagatattcatcaagaacactcccgtacctgccaatcctgcgacaaccttcactgcagccagagtggactctttcatcacacaatacagcaccggccaggtcgctgttcagattgaatgggaaattgaaaaggaacgctccaaacgctggaatcctgaagtgcagtttacttcaaactatgggaaccagtcttctatgttgtgggctcctgatacaactgggaagtatacagagccgcgggttattggctctcgttatttgactaatcatttgtaa

SEQ ID NO: 20 - DNA, 4213 bp, Dependoparvovirus Adeno-associated virus - 12
  source 1..4213: mol_type = other DNA
ttgcgacagtttgcgacaccatgtggtcacaagaggtatataaccgcgagtgagccagcgaggagctccattttgcccgcgaagtttgaacgagcagcagccatgccggggttctacgaggtggtgatcaaggtgcccagcgacctggacgagcacctgcccggcatttctgactcctttgtgaactgggtggccgagaaggaatgggagttgcccccggattctgacatggatcagaatctgattgagcaggcacccctgaccgtggccgagaagctgcagcgcgagttcctggtggaatggcgccgagtgagtaaatttctggaggccaagttttttgtgcagtttgaaaagggggactcgtactttcatttgcatattctgattgaaattaccggcgtgaaatccatggtggtgggccgctacgtgagtcagattagggataaactgatccagcgcatctaccgcggggtcgagccccagctgcccaactggttcgcggtcacaaagacccgaaatggcgccggaggcgggaacaaggtggtggacgagtgctacatccccaactacctgctccccaaggtccagcccgagcttcagtgggcgtggactaacatggaggagtatataagcgcctgtttgaacctcgcggagcgtaaacggctcgtggcgcagcacctgacgcacgtctcccagacccaggagggcgacaaggagaatctgaacccgaattctgacgcgccggtgatccggtcaaaaacctccgccaggtacatggagctggtcgggtggctggtggacaagggcatcacgtccgagaagcagtggatccaggaggaccaggcctcgtacatctccttcaacgcggcctccaactcccggtcgcagatcaaggcggccctggacaatgcctccaaaatcatgagcctcaccaaaacggctccggactatctcatcgggcagcagcccgtgggggacattaccaccaaccggatctacaaaatcctggaactgaacgggtacgacccccagtacgccgcctccgtctttctcggctgggcccagaaaaagtttggaaagcgcaacaccatctggctgtttgggcccgccaccaccggcaagaccaacatcgcggaagccatcgcccacgcggtccccttctacggctgcgtcaactggaccaatgagaactttcccttcaacgactgcgtcgacaaaatggtgatttggtgggaggagggcaagatgaccgccaaggtcgtagagtccgccaaggccattctgggcggcagcaaggtgcgcgtggaccaaaaatgcaaggcctctgcgcagatcgaccccacccccgtgatcgtcacctccaacaccaacatgtgcgccgtgattgacgggaacagcaccaccttcgagcaccagcagcccctgcaggaccggatgttcaagtttgaactcacccgccgcctcgaccacgactttggcaaggtcaccaagcaggaagtcaaggactttttccggtgggcggctgatcacgtgactgacgtggctcatgagttttacgtcacaaagggtggagctaagaaaaggcccgccccctctgacgaggatataagcgagcccaagcggccgcgcgtgtcatttgcgcagccggagacgtcagacgcggaagctcccggagacttcgccgacaggtaccaaaacaaatgttctcgtcacgcgggtatgctgcagatgctctttccctgcaagacgtgcgagagaatgaatcagaattccaacgtctgcttcacgcacggtcagaaagattgcggggagtgctttcccgggtcagaatctcaaccggtttctgtcgtcagaaaaacgtatcagaaactgtgcatccttcatcagctccggggggcacccgagatcgcctgctctgcttgcgaccaactcaaccccgatttggacgattgccaatttgagcaataaatgactgaaatcaggtatggctgctgacggttatcttccagattggctcgaggacaacctctctgaaggcattcgcgagtggtgggcgctgaaacctggagctccacaacccaaggccaaccaacagcatcaggacaacggcaggggtcttgtgcttcctgggtacaagtacctcggacccttcaacggactcgacaagggagagccggtcaacgaggcagacgccgcggccctcgagcacgacaaggcctacgacaagcagctcgagcagggggacaacccgtatctcaagtacaaccacgccgacgccgagttccagcagcgcttggcgaccgacacctcttttgggggcaacctcgggcgagcagtcttccaggccaaaaagaggattctcgagcctctgggtctggttgaagagggcgttaaaacggctcctggaaagaaacgcccattagaaaagactccaaatcggccgaccaacccggactctgggaaggccccggccaagaaaaagcaaaaagacggcgaaccagccgactctgctagaaggacactcgactttgaagactctggagcaggagacggaccccctgagggatcatcttccggagaaatgtctcatgatgctgagatgcgtgcggcgccaggcggaaatgctgtcgaggcgggacaaggtgccgatggagtgggtaatgcctccggtgattggcattgcgattccacctggtcagagggccgagtcaccaccaccagcacccgaacctgggtcctacccacgtacaacaaccacctgtacctgcgaatcggaacaacggccaacagcaacacctacaacggattctccaccccctggggatactttgactttaaccgcttccactgccacttttccccacgcgactggcagcgactcatcaacaacaactggggactcaggccgaaatcgatgcgtgttaaaatcttcaacatacaggtcaaggaggtcacgacgtcaaacggcgagactacggtcgctaataaccttaccagcacggttcagatctttgcggattcgacgtatgaactcccatacgtgatggacgccggtcaggaggggagctttcctccgtttcccaacgacgtctttatggttccccaatacggatactgcggagttgtcactggaaaaaaccagaaccagacagacagaaatgccttttactgcctggaatactttccatcccaaatgctaagaactggcaacaattttgaagtcagttaccaatttgaaaaagttcctttccattcaatgtacgcgcacagccagagcctggacagaatgatgaatcctttactggatcagtacctgtggcatctgcaatcgaccactaccggaaattcccttaatcaaggaacagctaccaccacgtacgggaaaattaccactggagactttgcctactacaggaaaaactggttgcctggagcctgcattaaacaacaaaaattttcaaagaatgccaatcaaaactacaagattcccgccagcgggggagacgcccttttaaagtatgacacgcataccactctaaatgggcgatggagtaacatggctcctggacctccaatggcaaccgcaggtgccggggactcggattttagcaacagccagctgatctttgccggacccaatccgagcggtaacacgaccacatcttcaaacaatttgttgtttacctcagaagaggagattgccacaacaaacccacgagacacggacatgtttggacagattgcagataataatcaaaatgccaccaccgcccctcacatcgctaacctggacgctatgggaattgttcccggaatggtctggcaaaacagagacatctactaccagggccctatttgggccaaggtccctcacacggacggacactttcacccttcgccgctgatgggaggatttggactgaaacacccgcctccacagattttcatcaaaaacacccccgtacccgccaatcccaatactacctttagcgctgcaaggattaattcttttctgacgcagtacagcaccggacaagttgccgttcagatcgactgggaaattcagaaggagcattccaaacgctggaatcccgaagttcaatttacttcaaactacggcactcaaaattctatgctgtgggctcccgacaatgctggcaactaccacgaactccgggctattgggtcccgtttcctcacccaccacttgtaa

SEQ ID NO: 21 - protein, 623 aa, Dependoparvovirus Adeno-associated virus - 1
MPGFYEIVIKVPSDLDEHLPGISDSFVSWVAEKEWELPPDSDMDLNLIEQAPLTVAEKLQRDFLVQWRRVSKAPEALFFVQFEKGESYFHLHILVETTGVKSMVLGRFLSQIRDKLVQTIYRGIEPTLPNWFAVTKTRNGAGGGNKVVDECYIPNYLLPKTQPELQWAWTNMEEYISACLNLAERKRLVAQHLTHVSQTQEQNKENLNPNSDAPVIRSKTSARYMELVGWLVDRGITSEKQWIQEDQASYISFNAASNSRSQIKAALDNAGKIMALTKSAPDYLVGPAPPADIKTNRIYRILELNGYEPAYAGSVFLGWAQKRFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLEHDFGKVTKQEVKEFFRWAQDHVTEVAHEFYVRKGGANKRPAPDDADKSEPKRACPSVADPSTSDAEGAPVDFADRYQNKCSRHAGMLQMLFPCKTCERMNQNFNICFTHGTRDCSECFPGVSESQPVVRKRTYRKLCAIHHLLGRAPEIACSACDLVNVDLDDCVSEQ

SEQ ID NO: 22 - protein, 536 aa, Dependoparvovirus Adeno-associated virus - 2
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 23 - protein, 621 aa, Dependoparvovirus Adeno-associated virus - 2
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 24 - protein, 312 aa, Dependoparvovirus Adeno-associated virus - 2
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 25 - protein, 397 aa, Dependoparvovirus Adeno-associated virus - 2
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 26 - protein, 624 aa, Dependoparvovirus Adeno-associated virus - 3
MPGFYEIVLKVPSDLDEHLPGISNSFVNWVAEKEWELPPDSDMDPNLIEQAPLTVAEKLQREFLVEWRRVSKAPEALFFVQFEKGETYFHLHVLIETIGVKSMVVGRYVSQIKEKLVTRIYRGVEPQLPNWFAVTKTRNGAGGGNKVVDDCYIPNYLLPKTQPELQWAWTNMDQYLSACLNLAERKRLVAQHLTHVSQTQEQNKENQNPNSDAPVIRSKTSARYMELVGWLVDRGITSEKQWIQEDQASYISFNAASNSRSQIKAALDNASKIMSLTKTAPDYLVGSNPPEDITKNRIYQILELNGYDPQYAASVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIEPTPVIVTSNTNMCAVIDGNSTTFEHQQPLQDRMFKFELTRRLDHDFGKVTKQEVKDFFRWASDHVTDVAHEFYVRKGGAKKRPASNDADVSEPKRQCTSLAQPTTSDAEAPADYADRYQNKCSRHVGMNLMLFPCKTCERMNQISNVCFTHGQRDCGECFPGMSESQPVSVVKKKTYQKLCPIHHILGRAPEIACSACDLANVDLDDCVSEQ

SEQ ID NO: 27 - protein, 623 aa, Dependoparvovirus Adeno-associated virus - 4
MPGFYEIVLKVPSDLDEHLPGISDSFVSWVAEKEWELPPDSDMDLNLIEQAPLTVAEKLQREFLVEWRRVSKAPEALFFVQFEKGDSYFHLHILVETVGVKSMVVGRYVSQIKEKLVTRIYRGVEPQLPNWFAVTKTRNGAGGGNKVVDDCYIPNYLLPKTQPELQWAWTNMDQYISACLNLAERKRLVAQHLTHVSQTQEQNKENQNPNSDAPVIRSKTSARYMELVGWLVDRGITSEKQWIQEDQASYISFNAASNSRSQIKAALDNASKIMSLTKTAPDYLVGQNPPEDISSNRIYRILEMNGYDPQYAASVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTKRLEHDFGKVTKQEVKDFFRWASDHVTEVTHEFYVRKGGARKRPAPNDADISEPKRACPSVAQPSTSDAEAPVDYADRYQNKCSRHVGMNLMLFPCRQCERMNQNVDICFTHGVMDCAECFPVSESQPVSVVRKRTYQKLCPIHHIMGRAPEVACSACELANVDLDDCDMEQ

SEQ ID NO: 28 - protein, 734 aa, Dependoparvovirus Adeno-associated virus - 4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 29 - protein, 610 aa, Dependoparvovirus Adeno-associated virus - 5
MATFYEVIVRVPFDVEEHLPGISDSFVDWVTGQIWELPPESDLNLTLVEQPQLTVADRIRRVFLYEWNKFSKQESKFFVQFEKGSEYFHLHTLVETSGISSMVLGRYVSQIRAQLVKVVFQGIEPQINDWVAITKVKKGGANKVVDSGYIPAYLLPKVQPELQWAWTNLDEYKLAALNLEERKRLVAQFLAESSQRSQEAASQREFSADPVIKSKTSQKYMALVNWLVEHGITSEKQWIQENQESYLSFNSTGNSRSQIKAALDNATKIMSLTKSAVDYLVGSSVPEDISKNRIWQIFEMNGYDPAYAGSILYGWCQRSFNKRNTVWLYGPATTGKTNIAEAIAHTVPFYGCVNWTNENFPFNDCVDKMLIWWEEGKMTNKVVESAKAILGGSKVRVDQKCKSSVQIDSTPVIVTSNTNMCVVVDGNSTTFEHQQPLEDRMFKFELTKRLPPDFGKITKQEVKDFFAWAKVNQVPVTHEFKVPRELAGTKGAEKSLKRPLGDVTNTSYKSLEKRARLSFVPETPRSSDVTVDPAPLRPLNWNSRYDCKCDYHAQFDNISNKCDECEYLNRGKNGCICHNVTHCQICHGIPPWEKENLSDFGDFDDANKEQ

SEQ ID NO: 30 - protein, 623 aa, Dependoparvovirus Adeno-associated virus - 6
MPGFYEIVIKVPSDLDEHLPGISDSFVNWVAEKEWELPPDSDMDLNLIEQAPLTVAEKLQRDFLVQWRRVSKAPEALFFVQFEKGESYFHLHILVETTGVKSMVLGRFLSQIRDKLVQTIYRGIEPTLPNWFAVTKTRNGAGGGNKVVDECYIPNYLLPKTQPELQWAWTNMEEYISACLNLAERKRLVAHDLTHVSQTQEQNKENLNPNSDAPVIRSKTSARYMELVGWLVDRGITSEKQWIQEDQASYISFNAASNSRSQIKAALDNAGKIMALTKSAPDYLVGPAPPADIKTNRIYRILELNGYDPAYAGSVFLGWAQKRFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLEHDFGKVTKQEVKEFFRWAQDHVTEVAHEFYVRKGGANKRPAPDDADKSEPKRACPSVADPSTSDAEGAPVDFADRYQNKCSRHAGMLQMLFPCKTCERMNQNFNICFTHGTRDCSECFPGVSESQPVVRKRTYRKLCAIHHLLGRAPEIACSACDLVNVDLDDCVSEQ

SEQ ID NO: 31 - protein, 623 aa, Dependoparvovirus Adeno-associated virus - 7
MPGFYEIVIKVPSDLDEHLPGISDSFVNWVAEKEWELPPDSDMDLNLIEQAPLTVAEKLQRDFLVQWRRVSKAPEALFFVQFEKGESYFHLHVLVETTGVKSMVLGRFLSQIREKLVQTIYRGVEPTLPNWFAVTKTRNGAGGGNKVVDECYIPNYLLPKTQPELQWAWTNMEEYISACLNLAERKRLVAQHLTHVSQTQEQNKENLNPNSDAPVIRSKTSARYMELVGWLVDRGITSEKQWIQEDQASYISFNAASNSRSQIKAALDNAGKIMALTKSAPDYLVGPSLPADIKTNRIYRILELNGYDPAYAGSVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLEHDFGKVTKQEVKEFFRWASDHVTEVAHEFYVRKGGASKRPAPDDADISEPKRACPSVADPSTSDAEGAPVDFADRYQNKCSRHAGMIQMLFPCKTCERMNQNFNICFTHGVRDCLECFPGVSESQPVVRKKTYRKLCAIHHLLGRAPEIACSACDLVNVDLDDCVSEQ

SEQ ID NO: 32 - protein, 624 aa, Dependoparvovirus Adeno-associated virus - 8
MPGFYEIVIKVPSDLDEHLPGISDSFVNWVAEKEWELPPDSDMDRNLIEQAPLTVAEKLQRDFLVQWRRVSKAPEALFFVQFEKGESYFHLHVLVETTGVKSMVLGRFLSQIREKLGPDHLPAGSSPTLPNWFAVTKDAVMAPAGGNKVVDECYIPNYLLPKTQPELQWAWTNMEEYISACLNLAERKRLVAQHLTHVSQTQEQNKENLNPNSDAPVIRSKTSARYMELVGWLVDRGITSEKQWIQEDQASYISFNAASNSRSQIKAALDNAGKIMALTKSAPDYLVGPSLPADITQNRIYRILALNGYDPAYAGSVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLEHDFGKVTKQEVKEFFRWASDHVTEVAHEFYVRKGGASKRPAPDDADKSEPKRACPSVADPSTSDAEGAPVDFADRYQNKCSRHAGMLQMLFPCKTCERMNQNFNICFTHGVRDCSECFPGVSESQPVVRKRTYRKLCAIHHLLGRAPEIACSACDLVNVDLDDCVSE

SEQ ID NO: 33 - protein, 622 aa, Dependoparvovirus Adeno-associated virus - 10
MPGFYEIVIKVPSDLDEHLPGISDSFVNWVAEKEWELPPDSDMDRNLIEQAPLTVAEKLQRDFLVHWRRVSKAPEALFFVQFEKGESYFHLHVLVETTGVKSMVLGRFLSQIRDRLVQTIYRGVEPTLPNWFAVTKTRNGAGGGNKVVDECYIPNYLLPKTQPELQWAWTNMEEYISACLNLAERKRLVAQHLTHVSQTQEQNKENLNPNSDAPVIRSKTSARYMELVGWLVDRGITSEKQWIQEDQASYISFNAASNSRSQIKAALDNAGKIMALTKSAPDYLVGPSLPADIKANRIYRILELNGYDPAYAGSVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLEHDFGKVTKQEVKEFFRWAQDHVTEVTHEFYVRKGGATKRPAPSDADISEPKRACPSVAEPSTSDAEAPVDFADRYQNKCSRHAGMLQMLFPCKTCERMNQNFNVCFTHGVRDCSECFPGASESQPVVRKKTYQKLCAIHHLLGRAPEIACSACDLVNVDLDDCVSEQ

SEQ ID NO: 34 - protein, 622 aa, Dependoparvovirus Adeno-associated virus - 11
MPGFYEIVIKVPSDLDEHLPGISDSFVNWVAEKEWELPPDSDMDRNLIEQAPLTVAEKLQRDFLVHWRRVSKAPEALFFVQFEKGESYFHLHVLVETTGVKSMVLGRFLSQIRDRLVQTIYRGVEPTLPNWFAVTKTRNGAGGGNKVVDECYIPNYLLPKTQPELQWAWTNMEEYISACLNLAERKRLVAQHLTHVSQTQEQNKENLNPNSDAPVIRSKTSARYMELVGWLVDRGITSEKQWIQEDQASYISFNAASNSRSQIKAALDNAGKIMALTKSAPDYLVGPSLPADIKANRIYRILELNGYDPAYAGSVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLEHDFGKVTKQEVKEFFRWAQDHVTEVAHEFYVRKGGATKRPAPSDADISEPKRACPSVPEPSTSDAEAPVDFADRYQNKCSRHAGMLQMLFPCKTCERMNQNFNVCFTHGVRDCSECFPGASESQPVVRKKTYQKLCAIHHLLGRAPEIACSACDLVNVDLDDCVSEQ

SEQ ID NO: 35 - protein, 621 aa, Dependoparvovirus Adeno-associated virus - 12
MPGFYEVVIKVPSDLDEHLPGISDSFVNWVAEKEWELPPDSDMDQNLIEQAPLTVAEKLQREFLVEWRRVSKFLEAKFFVQFEKGDSYFHLHILIEITGVKSMVVGRYVSQIRDKLIQRIYRGVEPQLPNWFAVTKTRNGAGGGNKVVDECYIPNYLLPKVQPELQWAWTNMEEYISACLNLAERKRLVAQHLTHVSQTQEGDKENLNPNSDAPVIRSKTSARYMELVGWLVDKGITSEKQWIQEDQASYISFNAASNSRSQIKAALDNASKIMSLTKTAPDYLIGQQPVGDITTNRIYKILELNGYDPQYAASVFLGWAQKKFGKRNTIWLFGPATTGKTNIAEAIAHAVPFYGCVNWTNENFPFNDCVDKMVIWWEEGKMTAKVVESAKAILGGSKVRVDQKCKASAQIDPTPVIVTSNTNMCAVIDGNSTTFEHQQPLQDRMFKFELTRRLDHDFGKVTKQEVKDFFRWAADHVTDVAHEFYVTKGGAKKRPAPSDEDISEPKRPRVSFAQPETSDAEAPGDFADRYQNKCSRHAGMLQMLFPCKTCERMNQNSNVCFTHGQKDCGECFPGSESQPVSVVRKTYQKLCILHQLRGAPEIACSACDQLNPDLDDCQFEQ

SEQ ID NO: 36 - protein, 736 aa, Dependoparvovirus Adeno-associated virus - 1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 37 - protein, 735 aa, Dependoparvovirus Adeno-associated virus - 2 [sequence omitted]

SEQ ID NO: 38 - protein, 598 aa, Dependoparvovirus Adeno-associated virus - 2
MAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 39 - protein, 533 aa, Dependoparvovirus Adeno-associated virus - 2
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 40 - protein, 736 aa, Dependoparvovirus Adeno-associated virus - 3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 41 - protein, 724 aa, Dependoparvovirus Adeno-associated virus - 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 42 - protein, 736 aa, Dependoparvovirus Adeno-associated virus - 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 43 - protein, 737 aa, Dependoparvovirus Adeno-associated virus - 7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 44 - protein, 737 aa, Dependoparvovirus Adeno-associated virus - 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRN

SEQ ID NO: 45 - protein, 736 aa, Dependoparvovirus Adeno-associated virus - 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 46 - protein, 738 aa, Dependoparvovirus Adeno-associated virus - 10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGESESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQANTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 47 - protein, 733 aa, Dependoparvovirus Adeno-associated virus - 11
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 48 - protein, 742 aa, Dependoparvovirus Adeno-associated virus - 12
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 49 - DNA, 1625 bp, synthetic construct
  misc_feature 1..1625: note = WT parkin construct
  source 1..1625: mol_type = other DNA
cctgggcaacgtgctggttattgtgctgtctcatcattttggcaaagaattcctcgaagatccgaaggggttcaagcttatcatcatgatagtgtttgtcaggttcaactccagccatggtttcccagtggaggtcgattctgacaccagcatcttccagctcaaggaggtggttgctaagcgacagggggttccggctgaccagttgcgtgtgattttcgcagggaaggagctgaggaatgactggactgtgcagaattgtgacctggatcagcagagcattgttcacattgtgcagagaccgtggagaaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgacagcaggaaggactcaccaccagctggaagtccagcaggtagatcaatctacaacagcttttatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacatcagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgttgggaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactggttcgacgtgtaaagtcgacaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 50 - DNA, 1625 bp, synthetic construct
  misc_feature 1..1625: note = 183Y construct
  source 1..1625: mol_type = other DNA
cctgggcaacgtgctggttattgtgctgtctcatcattttggcaaagaattcctcgaagatccgaaggggttcaagcttatcatcatgatagtgtttgtcaggttcaactccagccatggtttcccagtggaggtcgattctgacaccagcatcttccagctcaaggaggtggttgctaagcgacagggggttccggctgaccagttgcgtgtgattttcgcagggaaggagctgaggaatgactggactgtgcagaattgtgacctggatcagcagagcattgttcacattgtgcagagaccgtggagaaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgacagcaggaaggactcaccaccagctggaagtccagcaggtagatcaatctacaacagcttttatgtgtattgcaaaggcccctgtcaaagagtgcagccgggaaaactcagggtacagtgcagcacctgcaggcaggcaacgctcaccttgacccagggtccatcttgctacgatgatgttttaattccaaaccggatgagtggtgaatgccaatccccacactgccctgggactagtgcagaatttttctttaaatgtggagcacaccccacctctgacaaggaaacatcagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgttgggaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactggttcgacgtgtaaagtcgacaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 51 - DNA, 1625 bp, synthetic construct
  misc_feature 1..1625: note = 208Y construct
  source 1..1625: mol_type = other DNA
cctgggcaacgtgctggttattgtgctgtctcatcattttggcaaagaattcctcgaagatccgaaggggttcaagcttatcatcatgatagtgtttgtcaggttcaactccagccatggtttcccagtggaggtcgattctgacaccagcatcttccagctcaaggaggtggttgctaagcgacagggggttccggctgaccagttgcgtgtgattttcgcagggaaggagctgaggaatgactggactgtgcagaattgtgacctggatcagcagagcattgttcacattgtgcagagaccgtggagaaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgacagcaggaaggactcaccaccagctggaagtccagcaggtagatcaatctacaacagcttttatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatacttctttaaatgtggagcacaccccacctctgacaaggaaacatcagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgttgggaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactggttcgacgtgtaaagtcgacaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 52 - DNA, 1624 bp, synthetic construct
  misc_feature 1..1624: note = 463Y construct
  source 1..1624: mol_type = other DNA
cctgggcaacgtgctggttattgtgctgtctcatcattttggcaaagaattcctcgaagatccgaaggggttcaagcttatcatcatgatagtgtttgtcaggttcaactccagccatggtttcccagtggaggtcgattctgacaccagcatcttccagctcaaggaggtggttgctaagcgacagggggttccggctgaccagttgcgtgtgattttcgcagggaaggagctgaggaatgactggactgtgcagaattgtgacctggatcagcagagcattgttcacattgtgcagagaccgtggagaaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgacagcaggaaggactcaccaccagctggaagtccagcaggtagatcaatctacaacagcttttatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacatcagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagaccaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgttgggaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactggtacgacgtgtaaagtcgacaacttgtttattgcagcttataatggttacaaataaagcaatagcatcacaaatttcacaaataaagcatttttttcactgcattctagttgtggtttgtccaaactcatcaatgtatcttatcatgtctggatc

(19) United States
(12) Patent Application Publication
(10) Pub. No.: US 2025/0049958 A1
(43) Pub. Date: Feb. 13, 2025

(54) GENE THERAPY DELIVERY OF PARKIN MUTANTS HAVING INCREASED ACTIVITY TO TREAT PARKINSON'S DISEASE

(71) Applicant: NysnoBio GT Neurology, LLC, Mill Valley, CA (US)

(72) Inventor: Jennifer JOHNSTON, Mill Valley, CA (US)

(21) Appl. No.: 18/901,912

(22) Filed: Sep. 30, 2024

Related U.S. Application Data
(63) Continuation of application No. 17/327,562, filed on May 21, 2021, now Pat. No. 12,133,897.
(60) Provisional application No. 63/028,335, filed on May 21, 2020.

(51) Int. Cl. A61K 48/00

(52) U.S. Cl. CPC A61K 48/0058; A61K 48/0075

(57) ABSTRACT

The present disclosure provides novel gene therapy constructs comprising a PARK2 gene or activating variant and methods of administering to treat Parkinson's Disease or symptoms thereof.

### CROSS-REFERENCE TO RELATED APPLICATIONS

This application is a continuation of U.S. application Ser. No. 17/327,562, filed May 21, 2021, which claims priority to U.S. Provisional Application No. 63/028,335 filed May 21, 2020, the contents of each of which are herein incorporated by reference in their entireties.

### DESCRIPTION OF THE TEXT FILE SUBMITTED ELECTRONICALLY

The contents of the electronic sequence listing (NYSN_012_02US_SeqList_ST26.xml; Size: 135,691 bytes; and Date of Creation: Sep. 30, 2024) are herein incorporated by reference in its entirety.

### FIELD OF THE DISCLOSURE

The present disclosure relates generally to gene therapy for treatment of Parkinson's Disease. In particular, the disclosure provides compositions and methods for gene therapy in neurons.

### BACKGROUND OF THE INVENTION

The Ubiquitin-Proteasome Pathway System (UPS) is a critical pathway that regulates key regulator proteins and degrades misfolded or abnormal proteins. UPS is central to multiple cellular processes, and if defective or imbalanced, it leads to pathogenesis of a variety of diseases. Posttranslational modification of proteins by ubiquitin is a fundamental cellular mechanism that regulates protein stability and activity and underlies a multitude of functions, from almost every aspect of biology. The covalent attachment of ubiquitin to specific protein substrates is achieved through the action of E3 ubiquitin ligases. These ligases comprise over 500 different proteins and are categorized into multiple classes defined by the structural element of their E3 functional activity.

Parkin, which has been implicated in disorders of the central nervous system such as Parkinson's Disease (PD), is a RING-between-RING E3 ligase that functions in the covalent attachment of ubiquitin to specific substrates, and mutations in Parkin are linked to Parkinson's disease, cancer and mycobacterial infection. The individual RING domains for Parkin have been the subject of much debate, in regards to the specific residues that coordinate Zn ions, as well as their relationship to canonical RING crossbrace structures defining classical E2-binding domains. R0 is a novel domain structure, but is more similar to Zn-finger domains than to E3 RING domains (Riley et al. 2013. Nat Commun. 4:1982)

There have been few reports of successful gene therapy for Parkinson's Disease, and thus there is a need in the art for new gene therapy compositions and methods of treating and preventing Parkinson's Disease.

### SUMMARY OF THE INVENTION

The present disclosure is directed towards a novel approach of gene therapy to replace or repair a defective parkin gene (PARK2) in a patient with Parkinson's Disease to provide a therapeutic benefit.

In some aspects, the present disclosure provides methods of treating, preventing, or ameliorating a central nervous system disorder or a symptom thereof in a patient in need thereof, comprising administering a pharmaceutical composition comprising an AAV5 vector comprising a gene therapy construct comprising a parkin (PARK2) gene, wherein the method comprises administering the pharmaceutical composition to the brain of the patient. In some embodiments, the PARK2 gene is a variant PARK2 gene having at least 95% identity to the polynucleotide of SEQ ID NO: 1. In some embodiments, the variant PARK2 gene encodes for a variant parkin polypeptide having greater biological activity than a wild type parkin polypeptide. In some embodiments, the expressed variant parkin polypeptide demonstrates increased auto-ubiquitination compared to the wild-type parkin polypeptide of SEQ ID NO: 2. In some embodiments, the variant PARK2 gene encodes a polypeptide with a mutation at the amino acid position of 146, 183 and/or 463 of SEQ ID NO: 2. In some embodiments, the variant PARK2 gene encodes a polypeptide with a mutation at the amino acid position of 146 and/or 183 of SEQ ID NO: 2. In some embodiments, the mutation comprises substitution of the amino acid residue to a tyrosine residue. In some embodiments, the polypeptide comprises SEQ ID NO: 4, 6, or 8. In some embodiments, the variant PARK2 gene comprises the nucleic acid sequence of any one of SEQ ID NOs: 3, 5, and 7.

In some embodiments, expression of the PARK2 gene is under control of a tissue specific promoter. In some embodiments, expression of the PARK2 gene is under control of a neuron-specific promoter. In some embodiments, expression of the PARK2 gene is under control of a ubiquitous promoter. In some embodiments, the PARK2 gene is under control of a promoter selected from the list: chicken-beta-actin (CBA), human beta actin (HuBa), cytomegalovirus (cMV), CAG, PGL, EF1-alpha, GAPDFI, SV40, FIBV, human synapsin (hSYN1), alpha-internexin (INA), nestin (NES), tyrosine hydroxylase (TH), forkhead box A2 (FOXA2), calmodulin-dependent protein kinase II (CAMKII), and neuron-specific enolase (NSE).

In some embodiments, the pharmaceutical composition is administered by intrathecal administration. In some embodiments, the pharmaceutical composition is administered to the substantia nigra of the subject's brain.

In some embodiments, administration of the pharmaceutical composition results in expression of the parkin gene in neurons and glial cells. In some embodiments, the neurons are dopaminergic neurons or oligodendrocytes. In some embodiments, the glial cells are astrocytes. In some embodiments, administration of the pharmaceutical composition increases the number of dopaminergic neurons in the patient.

In some embodiments, the central nervous system disorder is Parkinson's Disease. In some embodiments, the patient displays one or more symptoms of Parkinson's Disease. In some embodiments, the patient is at risk of developing one or more symptoms of Parkinson's Disease. In some embodiments, the one or more symptoms of Parkinson's Disease is selected from the group consisting of: motor deficits, tremors, bradykinesia (slowed movement), rigid muscles, impaired posture and balance, loss of automatic movements, speech changes, writing changes, depression, swallowing problems, decreased cardiac function, sleep disorders, dementia, cognitive problems, emotional changes (e.g. fear, anxiety, or loss of motivation), blood pressure changes, fatigue, pain, involuntary movements, shuffling gait, dizziness, amnesia, confusion, voice box spasms, distorted sense of smell, jaw stiffness or reduced facial expression, and weight loss.

In some aspects, the present disclosure provides a recombinant gene therapy vector comprising a variant parkin (PARK2) gene packaged into an AAV5 capsid for administration to a patient in need thereof.

In some aspects, the present disclosure provides a recombinant nucleic acid molecule comprising a WT or mutant parkin (PARK2) sequence, and a sequence encoding an AAV capsid protein or portion thereof. In some embodiments, the sequence encodes an AAV capsid protein selected from AAV1, AAV2, AAV3, AAV4, AAV5, AAV6, AAV7, AAV8, AAV9, AAV10, AAV11, AAV12, or AAV13 capsid protein, or a mutant AAV capsid protein at least 95% identical to an AAV capsid protein selected from AAV1, AAV2, AAV3, AAV4, AAV5, AAV6, AAV7, AAV8, AAV9, AAV10, AAV11, AAV12, or AAV13 capsid protein. In some embodiments, the sequence encoding an AAV capsid protein is selected from any one of SEQ ID NOs: 9-20. In some embodiments, the sequence encoding an AAV capsid protein or portion thereof encodes a vp1-3 capsid protein or fragment thereof. In some embodiments, the sequence encoding a vp1-3 capsid protein is selected from any one of SEQ ID NOs: 36-48. In some embodiments, the sequence encodes an AAV capsid protein at least 95% identical to an AAV5 capsid protein. In some embodiments, the sequence encodes SEQ ID NO: 13

In some embodiments, the recombinant nucleic acid molecule comprises a functional rep gene. In some embodiments, the rep gene is AAV2 rep gene.

In some embodiments, he WT or variant parkin (PARK2) sequence encodes for SEQ ID NO: 2 or a polypeptide with at least 95% sequence identity to SEQ ID NO: 2. In some embodiments, the PARK2 sequence encodes a polypeptide with a mutation at the amino acid position of 146, 183 and/or 463 of SEQ ID NO: 2. In some embodiments, the PARK2 sequence encodes a polypeptide with a mutation at the amino acid position of 146 or 183 of SEQ ID NO: 2. In some embodiments, the PARK2 sequence encodes for SEQ ID NO: 2.

In some aspects the present disclosure provides a cultured host cell comprising the recombinant nucleic acid molecule or gene therapy vector of the disclosure. In some embodiments, the recombinant nucleic acid molecule is a plasmid.

### BRIEF DESCRIPTION OF THE DRAWINGS

FIG. 1 shows an exemplary schematic of the gene therapy constructs disclosed herein.

FIG. 2 shows a plasmid map of a gene therapy construct comprising a wild type Parkin cDNA sequence.

FIG. 3 shows a plasmid map of a gene therapy construct comprising a W183Y Parkin mutant cDNA sequence.

FIG. 4 shows a plasmid map of a gene therapy construct comprising a F208Y Parkin mutant cDNA sequence.

FIG. 5 shows a plasmid map of a gene therapy construct comprising a F463Y Parkin mutant cDNA sequence.

FIG. 6 shows the structure of the parkin polypeptide in both the “OFF” and “ON” state, and that the W183Y, F208Y, and F463Y parkin mutants increase activity of the parkin polypeptide.

FIG. 7A-C. Panels A & C show the purified W183Y, F208Y, and F463Y mutant polypeptides demonstrate increased auto-ubiquitination. Panel B shows that cells expressing this mutant exhibit increased functional mitophagy activity as determined by a Tom20 assay compared to wild type.

FIG. 8 shows the crystal structure of the Parkin polypeptide as a ribbon diagram demonstrating the R0 and R2 hydrophobic interface and amino acid residues described herein.

FIG. 9 shows an exemplary vector construct encoding a Parkin gene.

FIG. 10A-B shows expression of wild type Parkin in Hela and Hela S3 cells.

FIG. 11 shows treatment with epoximicin restores the variant parkin polypeptides to wild type levels.

FIG. 12 shows the expression of wild type Parkin in rat neuronal cells.

FIG. 13A-C shows expression of Parkin variants in the rat neuronal cells. Panel A shows the expression of Parkin 5C3 across the W183Y, F463Y, and F208Y mutants administered at Dose 1, Dose 2, and Dose 3. Panel B shows the histology of Dose 2 (5.9−7.2×1013 gc/mL) for the variants. Panel C shows a comparison between expression of Parkin 5C3 and Prk8 across the mutants.

FIG. 14 shows the presence of perivascular cuffing with Dose 3 of the Parkin W183Y vector.

FIG. 15A-B shows the CV intact side of one of the animals injected with the wild type Parkin construct. Panel A and B show different views of case #4 (animal BV156).

FIG. 16 shows that the wild type Parkin construct transduced both neuronal and glial-like profiles. This sample is from Case #1 (BO935).

FIG. 17 shows sparse coverage of SN dopaminergic cells after administration with the wild type Parkin construct. This sample is from Case #3 (BO803).

FIG. 18 shows a visible tissue reaction detected in Case #2 (BL926) after administration with the wild type Parkin construct.

FIG. 19A-D show the histology of each of the four animals injected with Dose two of the wild type Parkin construct. Panel A is Case #1 (BO935); Panel B is Case #2 (BL926); Panel C is Case #3 (BO803); and Panel D is Case #4 (BV156).

### DETAILED DESCRIPTION

### Definitions

While the following terms are believed to be well understood by one of ordinary skill in the art, the following definitions are set forth to facilitate explanation of the presently disclosed subject matter.

Throughout the present specification, the terms “about” and/or “approximately” may be used in conjunction with numerical values and/or ranges. The term “about” is understood to mean those values near to a recited value. For example, “about 40 [units]” may mean within ±25% of 40 (e.g., from 30 to 50), within ±20%, ±15%, ±10%, ±9%, ±8%, ±7%, ±6%, ±5%, ±4%, ±3%, ±2%, ±1%, less than ±1%, or any other value or range of values therein or therebelow. Furthermore, the phrases “less than about [a value]” or “greater than about [a value]” should be understood in view of the definition of the term “about” provided herein. The terms “about” and “approximately” may be used interchangeably.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features. Although the open-ended term “comprising,” as a synonym of terms such as including, containing, or having, is used herein to describe and claim the disclosure, the present technology, or embodiments thereof, may alternatively be described using more limiting terms such as “consisting of” or “consisting essentially of” the recited ingredients.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “a” or “an” refers to one or more of that entity; for example, “a kinase inhibitor” refers to one or more kinase inhibitors or at least one kinase inhibitor. As such, the terms “a” (or “an”), “one or more” and “at least one” are used interchangeably herein. In addition, reference to “an inhibitor” by the indefinite article “a” or “an” does not exclude the possibility that more than one of the inhibitors is present, unless the context clearly requires that there is one and only one of the inhibitors.

As used herein, the verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “treating” means one or more of relieving, alleviating, delaying, reducing, reversing, improving, or managing at least one symptom of a condition in a subject. The term “treating” may also mean one or more of arresting, delaying the onset (i.e., the period prior to clinical manifestation of the condition) or reducing the risk of developing or worsening a condition.

An “effective amount” means the amount of a formulation according to the invention that, when administered to a patient for treating a state, disorder or condition is sufficient to effect such treatment. The “effective amount” will vary depending on the active ingredient, the state, disorder, or condition to be treated and its severity, and the age, weight, physical condition and responsiveness of the mammal to be treated.

The term “therapeutically effective” applied to dose or amount refers to that quantity of a compound or pharmaceutical formulation that is sufficient to result in a desired clinical benefit after administration to a patient in need thereof.

All weight percentages (i.e., “% by weight” and “wt. %” and w/w) referenced herein, unless otherwise indicated, are measured relative to the total weight of the pharmaceutical composition.

“Ubiquitin Proteasome Pathway System (UPS)” as used herein relates to the ubiquitin proteasome pathway, conserved from yeast to mammals, and is required for the targeted degradation of most short-lived proteins in the eukaryotic cell. Targets include cell cycle regulatory proteins, whose timely destruction is vital for controlled cell division, as well as proteins unable to fold properly within the endoplasmic reticulum. Ubiquitin modification is an ATP-dependent process carried out by three classes of enzymes. An “ubiquitin activating enzyme” (E1) forms a thio-ester bond with ubiquitin, a highly conserved 76-amino acid protein. This reaction allows subsequent binding of ubiquitin to a “ubiquitin conjugating enzyme” (E2), followed by the formation of an isopeptide bond between the carboxy-terminus of ubiquitin and a lysine residue on the substrate protein. The latter reaction requires a “ubiquitin ligase” (E3). E3 ligases can be single- or multi-subunit enzymes. In some cases, the ubiquitin-binding and substrate binding domains reside on separate polypeptides brought together by adaptor proteins or culling. Numerous E3 ligases provide specificity in that each can modify only a subset of substrate proteins. Further specificity is achieved by post-translational modification of substrate proteins, including, but not limited to, phosphorylation. Effects of mono-ubiquitination include changes in subcellular localization. However, multiple ubiquitination cycles resulting in a poly-ubiquitin chain are required for targeting a protein to the proteasome for degradation. The multisubunit 26S proteasome recognizes, unfolds, and degrades poly-ubiquitinated substrates into small peptides. The reaction occurs within the cylindrical core of the proteasome complex, and peptide bond hydrolysis employs a core threonine residue as the catalytic nucleophile. It has been shown that an additional layer of complexity, in the form of multi-ubiquitin chain receptors, may lie between the poly-ubiquitination and degradation steps. These receptors react with a subset of poly-ubiquitinated substrates, aiding in their recognition by the 26S proteasome, and thereby promoting their degradation. This pathway is not only important in cellular homeostasis, but also in human disease. Because ubiquitin/proteasome-dependent degradation is often employed in control of the cell division cycle and cell growth, researchers have found that proteasome inhibitors hold some promise of being developed into potential cancer therapeutic agents.

Protein degradation through the ubiquitin-proteasome system is the major pathway of non-lysosomal proteolysis of intracellular proteins. It plays important roles in a variety of fundamental cellular processes such as regulation of cell cycle progression, division, development and differentiation, apoptosis, cell trafficking, and modulation of the immune and inflammatory responses. The central element of this system is the covalent linkage of ubiquitin to targeted proteins, which are then recognized by the 26S proteasome, an adenosine triphosphate-dependent, multi-catalytic protease. Damaged, oxidized, or misfolded proteins as well as regulatory proteins that control many critical cellular functions are among the targets of this degradation process. Aberration of this system leads to the dysregulation of cellular homeostasis and the development of multiple diseases (Wang et al. Cell Mol Immunol. 2006 August; 3 (4): 255-61).

“Parkin ligase” or “Parkin” as used herein relates to a protein which in humans is encoded by the PARK2 gene. (Kitada T, Asakawa S, Hattori N, Matsumine H, Yamamura Y, Minoshima S, Yokochi M, Mizuno Y, Shimizu N (April 1998). “Mutations in the parkin gene cause autosomal recessive juvenile parkinsonism”. Nature 392 (6676): 605-608. doi: 10.1038/33416. PMID 9560156. Matsumine H, Yamamura Y, Hattori N, Kobayashi T, Kitada T, Yoritaka A, Mizuno Y (April 1998). “A microdeletion of D6S305 in a family of autosomal recessive juvenile parkinsonism (PARK2)”. Genomics 49 (1): 143-146. doi: 10.1006/geno.1997.5196. PMID 9570960. The protein is a component of a multiprotein E3 ubiquitin ligase complex which in turn is part of the ubiquitin-proteasome system that mediates the targeting of proteins for degradation. Mutations in the PARK2 gene are known to cause a familial form of Parkinson's disease known as autosomal recessive juvenile Parkinson's disease (AR-JP).

“Ligase” as used herein, is an enzyme that can catalyze the joining of two or more compounds or biomolecules by bonding them together with a new chemical bond. The “ligation” of the two usually with accompanying hydrolysis of a small chemical group dependent to one of the larger compounds or biomolecules, or the enzyme catalyzing the linking together of two compounds, e.g., enzymes that catalyze joining of groups C—O, C—S, C—N, etc. Ubiquitin-protein (E3) ligases are a large family of highly diverse enzymes selecting proteins for ubiquitination.

“Ub Ligases” are involved in disease pathogenesis for oncology, inflammation & infectious disease. E3 ligase belonging to the RING-between-RING (RBR) family of E3 ligases containing both canonical RING domains and a catalytic cysteine residue usually restricted to HECT E3 ligases; termed ‘RING/HECT hybrid’ enzymes. Mutations in Parkin linked to Parkinson's disease, cancer and mycobacterial infection. Parkin is recognized as a neuroprotective protein with a role in mitochondrial integrity. Human genetic data implicate loss of Parkin activity as a mechanism for pathogenesis of Parkinson's disease (PD).

“Zinc Finger (ZnF) Domain” as used herein relates to a protein structure characterized by coordinating zinc ions to stabilize the functional activity. ZnF stabilize the binding of Ub, Deubiquitinating Enzymes (DUBs), and Ligases (E3) in the UPS.

The present disclosure provides, in part, compositions and methods for treating, preventing, inhibiting, or delaying central nervous system degeneration. In particular, the present disclosure provides a gene therapy construct comprising a parkin (E3 ubiquitin protein ligase (PARK2)) gene or functional fragment or variant thereof, and methods of delivering the construct to a subject.

### Parkin

Parkin consists of a ubiquitin-like (Ubl) domain and a 60-amino acid linker followed by RING0, a zinc finger unique to parkin (13), and three additional zinc finger domains characteristic of the RBR family. Parkin plays a role in the machinery that breaks down and degrades proteins in the cells by tagging damaged and excess proteins with ubiquitin. Parkin may also trigger the destruction of dysfunctional mitochondria, act as a tumor suppressor protein, regulate synaptic vesicles from nerve cells, and enhance cell survival by suppressing both mitochondria-dependent and mitochondria-independent apoptosis.

In some embodiments, a parkin (PARK2) variant of the present disclosure comprises one or more nucleotide mutations. In some embodiments, the parkin (PARK2) variant comprising one or more nucleotide mutations encodes a polypeptide comprising one or more amino acid substitutions, deletions, or additions. In some embodiments, the one or more amino acid substitutions replaces the wild type residue with a tyrosine.

In some embodiments, the present disclosure provides a parkin (PARK2) variant comprising one or more nucleotide mutations in the ubiquitin-like domain. In some embodiments, the present disclosure provides a parkin (PARK2) variant comprising one or more nucleotide mutations in a domain that disrupts a zinc-finer. In some embodiments, the present disclosure provides a parkin (PARK2) variant comprising one or more nucleotide mutations in an area that disrupts the interface between two zinc-finger domains. In some embodiments, the present disclosure provides a parkin (PARK2) variant comprising one or more nucleotide mutations in the RING0, RING1, and/or RING2 domains. In some embodiments, the present disclosure provides a parkin (PARK2) variant comprising one or more nucleotide mutations in the Ubl, RING0, RING1, and/or RING2 domains.

In some embodiments, the parkin (PAK2) variant demonstrates increased activity compared to a wild type parkin (e.g. a polypeptide encoded by SEQ ID NO: 1). In some embodiments, the parkin (PARK2) variant demonstrates increased ubiquitination compared to ubiquitination by a wild type parkin. In some embodiments, the variant demonstrates increased auto-ubiquitination compared to a wild type parkin. In some embodiments, the variant demonstrates increased inhibition of parkin polypeptide activity compared to a wild type parkin. In some embodiments, the variant demonstrates increased auto-inhibition of parkin polypeptide activity compared to a wild type parkin.

In some embodiments, the variant parkin that demonstrates increased activity compared to a wild type parkin is a polypeptide having the amino acid sequence of any one of SEQ ID NOs: 4, 6, and 8. In some embodiments, the parkin variant that demonstrates increased auto-ubiquitination compared to wild type parkin is a polypeptide having the amino acid sequence of any one of SEQ ID NOs: 4, 6, and 8. In some embodiments, the PARK2 variant encoding a variant parkin polypeptide having increased activity compared to a wild type parkin has a polynucleotide sequence of any one of SEQ ID NOs: 3, 4, and 7. In some embodiments, the PARK2 variant encoding a variant parkin polypeptide having increased auto-ubiquitination compared to wild type parkin has a polynucleotide sequence of any one of SEQ ID NOs: 3, 5, and 7.

In some embodiments, the present disclosure provides a polynucleotide sequence at least 75% identical to any one of SEQ ID NOs: 1, 3, 5, and/or 7. In some embodiments, the present disclosure provides a polynucleotide sequence about 75% to about 99.9% identical to any one of SEQ ID NOs: 1, 3, 5, and/or 7. In some embodiments, the present disclosure provides a polynucleotide sequence about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 98%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% to any one of SEQ ID NOs: 1, 3, 5, and/or 7. In some embodiments, the polynucleotide is DNA, cDNA, or RNA. In some embodiments, the polynucleotide is codon-optimized.

In some embodiments, the present disclosure provides gene therapy constructs comprising a polynucleotide sequence of one or more of SEQ ID NOs: 1, 3, 5, and/or 7. In some embodiments, the present disclosure provides gene therapy constructs comprising a polynucleotide sequence at least 75% identical to one or more of SEQ ID NOs: 1, 3, 5, and/or 7. In some embodiments, the present disclosure provides a gene therapy construct comprising a polynucleotide sequence about 75% to about 99.9% identical to one or more of SEQ ID NOs: 1, 3, 5, and/or 7. In some embodiments, the present disclosure provides a gene therapy construct comprising a polynucleotide sequence about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 98%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% to one or more of SEQ ID NOs: 1, 3, 5, and/or 7. In some embodiments, the polynucleotide is DNA, cDNA, or RNA. In some embodiments, the polynucleotide is codon-optimized.

In some embodiments, the present disclosure provides a polynucleotide sequence of any one of SEQ ID NOs: 1, 3, 5, and/or 7 comprising one or more nucleotide substitutions, mutations, deletions, additions, and/or truncations. In some embodiments, between about 1-20 or more nucleotides are mutated, substituted, deleted, added, and/or truncated. In some embodiments, about 1, about 2, about 3, about 4, about 5, about 6, about 7, about 8, about 9, about 10, about 11, about 12, about 13, about 14, about 15, about 16, about 17, about 18, about 19, or about 20 or more nucleotides are mutated, substituted, deleted, added, and/or truncated. In some embodiments, the polynucleotide sequence is extended or truncated at only one end. In some embodiments, the polynucleotide sequence is extended or truncated at both ends. In some embodiments, the polynucleotide sequence is extended at one end and truncated at the other.

In some embodiments, the present disclosure provides a gene therapy construct comprising a polynucleotide sequence of one or more of SEQ ID NOs: 1, 3, 5, and/or 7 comprising one or more nucleotide substitutions, mutations, deletions, additions, and/or truncations. In some embodiments, between about 1-20 or more nucleotides are mutated, substituted, deleted, added, and/or truncated. In some embodiments, about 1, about 2, about 3, about 4, about 5, about 6, about 7, about 8, about 9, about 10, about 11, about 12, about 13, about 14, about 15, about 16, about 17, about 18, about 19, or about 20 or more nucleotides are mutated, substituted, deleted, added, and/or truncated. In some embodiments, the polynucleotide sequence is extended or truncated at only one end. In some embodiments, the polynucleotide sequence is extended or truncated at both ends. In some embodiments, the polynucleotide sequence is extended at one end and truncated at the other.

In some embodiments, the present disclosure provides a polynucleotide sequence of any of SEQ ID NOs: 49-52. In some embodiments, the present disclosure provides a polynucleotide sequence at least 75% identical to any one of SEQ ID NOs: 49-52. In some embodiments, the present disclosure provides a polynucleotide sequence about 75% to about 99.9% identical to any one of SEQ ID NOs: 49-52. In some embodiments, the present disclosure provides a polynucleotide sequence about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 98%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% to any one of SEQ ID NOs: 49-52. In some embodiments, the polynucleotide is DNA, cDNA, or RNA. In some embodiments, the polynucleotide is codon-optimized.

In some embodiments, the present disclosure provides gene therapy constructs comprising a polynucleotide sequence of one or more of SEQ ID NOs: 49-52. In some embodiments, the present disclosure provides gene therapy constructs comprising a polynucleotide sequence at least 75% identical to one or more of SEQ ID NOs: 49-52. In some embodiments, the present disclosure provides a gene therapy construct comprising a polynucleotide sequence about 75% to about 99.9% identical to one or more of SEQ ID NOs: 49-52. In some embodiments, the present disclosure provides a gene therapy construct comprising a polynucleotide sequence about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 98%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% to one or more of SEQ ID NOs: 49-52. In some embodiments, the polynucleotide is DNA, cDNA, or RNA. In some embodiments, the polynucleotide is codon-optimized.

In some embodiments, the present disclosure provides a polypeptide having the amino acid sequence of any one of SEQ ID NOs: 2, 4, 6, and/or 8. In some embodiments, the present disclosure provides a polypeptide having an amino acid sequence at least 75% identical to any one of SEQ ID NOs: 2, 4, 6, and/or 8. In some embodiments, the present disclosure provides a polypeptide having an amino acid sequence about 75% to about 99.9% identical to any one of SEQ ID NOs: 2, 4, 6, and/or 8. In some embodiments, the present disclosure provides a polypeptide having an amino acid sequence about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 98%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% to any one of SEQ ID NOs: 2, 4, 6, and/or 8.

In some embodiments, the present disclosure provides a gene therapy construct comprising a parkin gene encoding the amino acid sequence of one or more of SEQ ID NOs: 2, 4, 6, and/or 8. In some embodiments, the present disclosure provides a gene therapy construct comprising a parkin gene encoding an amino acid sequence at least 75% identical to one or more of SEQ ID NOs: 2, 4, 6, and/or 8. In some embodiments, the present disclosure provides a gene therapy construct comprising a parkin gene encoding an amino acid sequence about 75% to about 99.9% identical to one or more of SEQ ID NOs: 2, 4, 6, and/or 8. In some embodiments, the present disclosure provides a gene therapy construct comprising a parkin gene encoding an amino acid sequence about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 98%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% to one or more of SEQ ID NOs: 2, 4, 6, and/or 8. In some embodiments, the present disclosure provides a polypeptide having the amino acid sequence of any one of SEQ ID NOs: 2, 4, 6, and/or 8 comprising one or more amino acid substitutions, mutations, deletions, additions, and/or truncations. In some embodiments, between about 1 and 7 or more amino acid residues are substituted, mutated, deleted, added, and/or truncated. In some embodiments, about 1, about 2, about 3, about 4, about 5, about 6, about 7, or more amino acid residues are substituted, mutated, deleted, added, and/or truncated. In some embodiments, the amino acid sequence is extended or truncated at the C-terminus. In some embodiments, the amino acid sequence is extended or truncated at the N-terminus. In some embodiments, the amino acid sequence is extended or truncated at both the N- and the C-terminus. In some embodiments, the amino acid sequence is extended at the N-terminus and truncated at both the C-terminus. In some embodiments, the amino acid sequence is extended at the C-terminus and truncated at both the N-terminus.

In some embodiments, the present disclosure provides a gene therapy construct comprising a parkin gene encoding the amino acid sequence of one or more of SEQ ID NOs: 2, 4, 6, and/or 8 comprising one or more amino acid substitutions, mutations, deletions, additions, and/or truncations. In some embodiments, between about 1 and 7 or more amino acid residues are substituted, mutated, deleted, added, and/or truncated. In some embodiments, about 1, about 2, about 3, about 4, about 5, about 6, about 7, or more amino acid residues are substituted, mutated, deleted, added, and/or truncated. In some embodiments, the amino acid sequence is extended or truncated at the C-terminus. In some embodiments, the amino acid sequence is extended or truncated at the N-terminus. In some embodiments, the amino acid sequence is extended or truncated at both the N- and the C-terminus. In some embodiments, the amino acid sequence is extended at the N-terminus and truncated at both the C-terminus. In some embodiments, the amino acid sequence is extended at the C-terminus and truncated at both the N-terminus.

In some embodiments, the present disclosure provides a parkin (PARK2) variant polypeptide comprising a mutation altering at least one amino acid residue to a tyrosine of wild type Parkin (e.g. the polypeptide encoded by SEQ ID NO: 1). In some embodiments, the present disclosure provides a parkin (PARK2) variant polypeptide comprising at least one phenylanine and/or tryptophan residue substituted with a tyrosine of wild type Parkin. In some embodiments, the present disclosure provides a parkin (PARK2) variant polypeptide comprising mutations at one or more of the 146, 208, 183, 403, 457, and/or 463 residues of wild type Parkin. In some embodiments, the present disclosure provides a parkin (PARK2) variant polypeptide comprising mutations at F146, W183, F208, W403, C457, W462, and/or F463 of wild type Parkin. In some embodiments, the present disclosure provides a parkin (PARK2) variant polypeptide comprising one or more of F146A or F146Y, W183Y, F208Y, W403A, and/or F463Y of wild type Parkin. In some embodiments, the present disclosure provides a parkin (PARK2) variant polypeptide comprising one or more of W183Y, F208Y, and/or F463Y of wild type Parkin.

In some embodiments, the present disclosure provides a parkin (PARK2) variant polypeptide comprising W183A, W183R, W183N, W183D, W183C, W183E, W183Q, W183G, W183H, W183I, W183L, W183K, W183M, W183F, W183P, W183S, W183T, W183Y or W183V of wild type Parkin.

In some embodiments, the present disclosure provides a parkin (PARK2) variant polypeptide comprising F208A, F208R, F208N, F208D, F208C, F208E, F208Q, F208G, F208H, F2081, F208L, F208K, F208M, F208P, F208S, F208T, F208W, F208Y or F208V of wild type Parkin. In some embodiments, the present disclosure provides a parkin (PARK2) variant polypeptide comprising F463A, F463R, F463N, F463D, F463C, F463E, F463Q, F463G, F463H, F463I, F463L, F463K, F463M, F463P, F463S, F463T, F463W, F463Y or F463V of wild type Parkin.

### Recombinant Gene Therapy Constructs

The recombinant gene therapy construct of the present disclosure may include gene regulatory elements. In some embodiments, the recombinant gene therapy construct comprises a polynucleotide comprising, in the following 5′ to 3′ order, a eukaryotically active promoter sequence and the sequence encoding the wild-type parkin, or functional fragment or variant thereof. The sequence encoding the wild-type parkin, or functional fragment or variant thereof, is operably linked to the eukaryotically active promoter sequence.

In some embodiments, the present disclosure provides polynucleotide sequences comprising a polynucleotide sequence of one or more of SEQ ID NOs: 49-52 or variants thereof as disclosed herein. In some embodiments, the present disclosure provides gene therapy constructs comprising a polynucleotide sequence of one or more of SEQ ID NOs: 49-52 or variants thereof as disclosed herein.

In some embodiments, the recombinant gene therapy construct comprises a minigene. In some embodiments, the minigene comprises, at a minimum, a parkin or variant parkin as disclosed herein, and its regulatory sequences, and AAV inverted terminal repeats (ITRs).

In some embodiments, the recombinant gene therapy construct comprises one or more regulatory elements, which may be operably linked to the parkin or variant parkin nucleotide sequence to permit transcription, translation, and/or expression in a cell transfected with the plasmid vector or infected with the virus produced using the methods disclosed herein.

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. Expression control sequences, including promoters which are native, constitutive, inducible and/or tissue-specific, are known in the art and may be utilized.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer, the SV40 promoter, the dihydrofolate reductase promoter, the B-actin promoter, the phosphoglycerol kinase (PGK) promoter, and the EF1α promoter. In some embodiments, the recombinant gene therapy construct comprises a ubiquitous promotor selected from the group including but not limited to, CMV, CAG, UBC, PGK, EF1-alpha, GAPDH, SV40, HBV, and chicken beta-actin (CBA) promoters.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system; the ecdysone insect promoter, the tetracycline-repressible system, the tetracycline-inducible system, the RU486-inducible system and the rapamycin-inducible system. Still other types of inducible promoters which may be useful in this context are those which are regulated by a specific physiological state, e.g., temperature, acute phase, a particular differentiation state of the cell, or in replicating cells only.

In some embodiments, the native promoter for the parkin gene is used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the promoter is a tissue-specific promoter. In some embodiments, the promoter is a neuronal or glial specific promoter. In some embodiments, the recombinant gene therapy construct comprises one or more of a neuron-specific promoter, including but not limited to, hSYN1 (human synapsin), INA (alpha-internexin), NES (nestin), TH (tyrosine hydroxylase), FOXA2 (Forkhead box A2), CaMKII (calmodulin-dependent protein kinase II), and NSE (neuron-specific enolase) promoters.

In some embodiments, the recombinant gene therapy construct comprises an enhancer. In some embodiments, the enhancer is derived from a virus or bacteria. In some embodiments, the enhancer is derived from cytomegalovirus (CMV). In some embodiments, the enhancer is CMV.IE.

In some embodiments, the recombinant gene therapy construct comprises an intron. In some embodiments, the intron is derived from a beta-actin. In some embodiments, the intron is a CBA intron. In some embodiments, the recombinant gene therapy construct comprises a poly-A signal. In some embodiments, the poly-A signal is an early poly-A sequence. In some embodiments, poly-A sequence is an SV40 early poly-A sequence.

In some embodiments, the recombinant gene therapy construct of the present disclosure comprises one or more selectable markers or reporter genes may include sequences encoding geneticin, hygromicin or purimycin resistance, among others.

The minigene can be carried on any suitable vector, e.g., a plasmid, which is delivered to a host cell. The plasmids useful in this invention may be engineered such that they are suitable for replication and, optionally, integration in prokaryotic cells, mammalian cells, or both. These plasmids (or other vectors carrying the 5′ AAV ITR-heterologous molecule-3′ITR) contain sequences permitting replication of the minigene in eukaryotes and/or prokaryotes and selection markers for these systems. Selectable markers or reporter genes may include sequences encoding geneticin, hygromicin or purimycin resistance, among others. The plasmids may also contain certain selectable reporters or marker genes that can be used to signal the presence of the vector in bacterial cells, such as ampicillin resistance. Other components of the plasmid may include an origin of replication and an amplicon, such as the amplicon system employing the Epstein Barr virus nuclear antigen. This amplicon system, or other similar amplicon components permit high copy episomal replication in the cells. Preferably, the molecule carrying the minigene is transfected into the cell, where it may exist transiently. Alternatively, the minigene may be stably integrated into the genome of the host cell, either chromosomally or as an episome. In some embodiments, the minigene may be present in multiple copies, optionally in head-to-head, head-to-tail, or tail-to-tail concatamers. Suitable transfection techniques are known and may readily be utilized to deliver the minigene to the host cell.

Generally, when delivering the vector comprising the minigene by transfection, the vector is delivered in an amount from about 5 μg to about 100 μg DNA, and preferably about 10 to about 50 μg DNA to about 1×10⁴ cells to about 1×10¹³ cells, and preferably about 10⁵ cells. However, the relative amounts of vector DNA to host cells may be adjusted, taking into consideration such factors as the selected vector, the delivery method and the host cells selected.

In some embodiments, the present disclosure provides host cell containing a polynucleotide sequence of one or more of SEQ ID NOs: 49-52 or variants thereof as disclosed herein. In some embodiments, the present disclosure provides host cell containing a polynucleotide sequence of one or more of SEQ ID NOs: 49-52 or variants thereof as disclosed herein.

### Adeno-Associated Virus Vectors

Adeno-associated virus (AAV) is a replication-deficient parvovirus possessing a single-stranded DNA genome that is about 4.7 kb in length and includes two 145-nucleotide inverted terminal repeat (ITRs). Cis-acting sequences directing viral DNA replication (rep), encapsidation/packaging and host cell chromosome integration are contained within the AAV ITRs. Three AAV promoters (named p5, p19, and p40) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep78, rep68, rep52, and rep40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells (e.g. gene therapy). AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA. To generate AAV vectors, the rep and cap proteins may be provided in trans. Moreover, AAV is extremely stable, including at conditions used to inactivate adenovirus (56° to 65° C. for several hours), thus making cold preservation of AAV less critical. AAV may even be lyophilized. Finally, AAV-infected cells are not resistant to superinfection.

AAV vectors are especially suited as a CNS gene therapy vector as they can mediate gene transfer to both mitotic and post-mitotic cells, are neurotropic, can exist stably in an episomal state, with little or no pathogenicity or cytotoxicity. AAV vectors are, advantageously, non-replicating vectors that rarely integrates into the host genome, and thus are generally the safest viral vector of choice for gene therapy to the brain. Further, the different serotypes of AAV vectors allow targeting of the gene therapy to the desired portions of the brain. For example, AAV 8 and AAV9 primarily target the neurons, and AAV4 preferentially infects astrocytes and ependymal cells.

In some embodiments, the gene therapy construct of the present disclosure comprise AAV sequences. In some embodiments, the gene therapy construct of the present disclosure is an AAV vector. In some embodiments, the AAV vector is of the serotype AAV1, AAV2, AAV4, AAV5, AAV6, AAV7, AAV8, AAV9, AAV10, AAV11, AAV12, AAV13, or AAVrhIO. In some embodiments, the AAV comprises a capsid protein having at least 95% identity to a wild-type VP1, VP2, or VP3 capsid protein. In some embodiments, the AAV vector or portion thereof is encoded by any one of SEQ ID NOs: 9-20 or a variant or fragment thereof. In some embodiments, the AAV vector or portion thereof is encoded by SEQ ID NO: 13 or a variant or fragment thereof.

In some embodiments, the present disclosure provides a polynucleotide sequence at least 75% identical to any one of SEQ ID NOs: 9-20 or a variant or fragment thereof. In some embodiments, the present disclosure provides a polynucleotide sequence about 75% to about 99.9% identical to any one of SEQ ID NOs: 9-20. In some embodiments, the present disclosure provides a polynucleotide sequence about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 98%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% to any one of SEQ ID NOs: 19-20. In some embodiments, the polynucleotide is DNA, cDNA, or RNA. In some embodiments, the polynucleotide is codon-optimized.

In some embodiments, the polynucleotide sequence encodes an AAV rep protein or a variant or fragment thereof. In some embodiments, the AAV rep protein is rep 40, rep 58, rep 68, or rep 78 or a variant or fragment thereof. In some embodiments, the rep protein is a polypeptide having the amino acid sequence of any one of SEQ ID NOs: 21-35 or a variant or fragment thereof. In some embodiments, the rep protein is a polypeptide having the amino acid sequence of SED ID NO: 29, or a variant or fragment thereof. In some embodiments, the present disclosure provides a polypeptide having an amino acid sequence at least 75% identical to any one of SEQ ID NOs: 21-35. In some embodiments, the present disclosure provides a polypeptide having an amino acid sequence about 75% to about 99.9% identical to any one of SEQ ID NOs: 21-35. In some embodiments, the present disclosure provides a polypeptide having an amino acid sequence about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 98%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% to any one of SEQ ID NOs: 21-35.

In some embodiments, the polynucleotide sequence encodes an AAV VP protein or a variant or fragment thereof. In some embodiments, the AAV VP protein is VP1 or VP3 or a variant or fragment thereof. In some embodiments, the VP protein is a polypeptide having the amino acid sequence of any one of SEQ ID NOs: 36-48 or a variant or fragment thereof. In some embodiments, the rep protein is a polypeptide having the amino acid sequence of SED ID NO: 41, or a variant or fragment thereof. In some embodiments, the present disclosure provides a polypeptide having an amino acid sequence at least 75% identical to any one of SEQ ID NOs: 36-48. In some embodiments, the present disclosure provides a polypeptide having an amino acid sequence about 75% to about 99.9% identical to any one of SEQ ID NOs: 36-48. In some embodiments, the present disclosure provides a polypeptide having an amino acid sequence about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 98%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% to any one of SEQ ID NOs: 36-48.

In some embodiments, the AAV vector is AAV5. In some embodiments, the AAV5 vector transduces both neurons and glial cells in the brain. In some embodiments, administration of an AAV5 vector comprising a parkin nucleotide sequence disclosed herein allows expression of the parkin or parkin variant polypeptide in both neurons and glial cells. In some embodiments, expression of the parkin or parkin variant polypeptide in both neurons and glial cells increases the therapeutic effect of the gene therapy compared with expression of parkin or a parkin variant polypeptide in neurons or glial cells alone. In some embodiments, the neurons are dopaminergic neurons. In some embodiments, the neurons are oligodendrocytes. In some embodiments, the glial cells are astrocytes.

### Methods of Treatment

Ubiquitination is crucial for a plethora of physiological processes, including cell survival and differentiation and innate and adaptive immunity. Proteins are built-up to cater for the structural and biochemical requirements of the cell and they are also broken-down in a highly regulated process serving more purposes than just destruction and space management. Proteins have different half-lives, determined by properties inherent to each protein substrate. Some will be long-lived, while other will rapidly be degraded. Proteolysis not only enables the cell to dispose of misfolded or damaged proteins, but also to fine-tune the concentration of essential proteins within the cell, such as the proteins involved in the cell cycle. This rapid, highly specific degradation can be achieved through the addition of at least four ubiquitin molecules to a target protein. Protein degradation is dependent on the post-translational addition of ubiquitin to the epsilon amino-group of specific lysine residues in a process called ubiquitination.

In recent years, considerable progress has been made in the understanding of the molecular action of ubiquitin in signaling pathways and how alterations in the ubiquitin system lead to the development of distinct human diseases. It has been shown that ubiquitination plays a role in the onset and progression of cancer, metabolic syndromes, neurodegenerative diseases, autoimmunity, inflammatory disorders, infection and muscle dystrophies (Popovic et al. Nature Medicine 20, 1242-1253 (2014)).

Ubiquitin-protein (E3) ligases are a large family of enzymes that select various proteins for ubiquitination. These ubiquitin ligases, called “Ub ligases” are known to have a role in various diseases and conditions, including but not limited to, cancer, inflammation and infectious diseases.

One specific Ub ligase is parkin ligase. Parkin ligase is a component of a multiprotein “E3” ubiquitin ligase complex, which in turn is part of the ubiquitin-proteasome system that mediates the targeting of proteins for degradation. Mutations in parkin ligase are linked to various diseases, such as Parkinson's disease, cancer and mycobacterial infection. Parkin ligase is thus an attractive target for therapeutic intervention.

In some aspects, the disclosure provides a method of inhibiting degeneration or death of a dopaminergic neuron comprising a mutation in a PARK2 gene associated with a Parkinson's Disease (PD). In some aspects, the method comprises contacting a neuron with a recombinant gene therapy construct comprising a polynucleotide encoding a wild-type parkin protein (e.g. SEQ ID NO: 2) expressed by a wild-type version of the PARK2 gene (e.g. SEQ ID NO: 1), or a functional variant or fragment thereof. Following contact with the recombinant gene therapy vector, the neuron expresses the wild-type protein, or functional variant or fragment thereof. In some embodiments, the functional variant of the wild type PARK2 gene is one or more of the polynucleotide sequences of SEQ ID NOs: 3, 5, and/or 7. In some embodiments, the functional variant of the wild type PARK2 gene is one or more of the variants of the polynucleotide sequences of any of SEQ ID NOs: 1, 3, 5, and 7 as disclosed herein. In some embodiments, the functional variant of the wild type PARK2 gene encodes a variant polypeptide having the amino acid sequence of any one of SEQ ID NOs: 2, 4, 6, and 8 or variants thereof as disclosed herein.

In some embodiments, administration of a gene construct of the present disclosure to a subject treats, prevents, delays, ameliorates, or reduces the incidence of one or more diseases or ailments selected from the group consisting of Alzheimer's Dementia, Parkinson's disease, Huntington Disease, Amyotrophic Lateral Sclerosis (ALS), Freidreich's ataxia, Spinocerebellar Ataxia, Multiple Systems Atrophy, PSP, Tauopathy, Diffuse Lewy Body Disease, Lewy Body dementia, any disorder characterized by abnormal accumulation of α-synuclein, disorders of the aging process, stroke, bacterial infection, viral infection, Mitochondrial related disease, mental retardation, deafness, blindness, diabetes, obesity, cardiovascular disease, multiple sclerosis, Sjogrens syndrome, lupus, glaucoma, including pseudoexfoliation glaucoma, Leber's Hereditary Optic Neuropathy, and rheumatoid arthritis.

In some embodiments, after transfection with a gene therapy construct of the instant disclosure, the transfected neuron and/or glial cell expresses a parkin or variant parkin polypeptide as disclosed herein. In some embodiments, after transfection with a gene therapy construct of the instant disclosure, the transfected neuron and/or glial cell expresses more parkin polypeptide as disclosed herein compared to that of a neuron and/or glial cell in a PARK2-deficient patient, or the neuron of the same patient before transfection. In some embodiments, the transfected neuron and/or glial cell expresses between about 1% to about 300% or more parkin or a functional variant thereof compared to that of a neuron and/or glial cell in a PARK2-deficient patient, or the neuron and/or glial cell of the same patient before transfection. In some embodiments, the transfected neuron and/or glial cell expresses about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 90%, about 95%, about 99%, about 100%, about 200%, about 300% or more parkin or a functional variant thereof compared to that of a neuron and/or glial cell in a PARK2-deficient patient, or the neuron and/or glial cell of the same patient before transfection. In some embodiments, the transfected neuron and/or glial cell expresses more parkin or a functional variant thereof compared to that of a neuron and/or glial cell in a PARK2-deficient patient, or the neuron and/or glial cell of the same patient before transfection for about 1 week to about 10 years or more. In some embodiments, the transfected neuron and/or glial cell expresses more parkin or a functional variant thereof compared to that of a neuron and/or glial cell in a PARK2-deficient patient, or the neuron and/or glial cell of the same patient before transfection for about 1 week, about 1 month, about 2 months, about 3 months, about 4 months, about 5 months, about 6 months, about 8 months, about 1 year, about 2 years, about 5 years, and/or about 10 years or more. In some embodiments, the transfected neuron and/or glial cell expresses about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 90%, about 95%, about 99%, about 100%, about 200%, about 300% or more parkin or a functional variant thereof compared to that of a neuron and/or glial cell in a PARK2-deficient patient, or the neuron and/or glial cell of the same patient before transfection for about 1 week, about 1 month, about 2 months, about 3 months, about 4 months, about 5 months, about 6 months, about 8 months, about 1 year, about 2 years, about 5 years, and/or about 10 years or more.

In some embodiments, after transfection with a gene therapy construct of the instant disclosure, the neuron and/or glial cell expressing a parkin polypeptide or a functional variant thereof as disclosed herein comprises a reduced amount of Lewy bodies compared to that of a neuron and/or glial cell in a PARK2-deficient patient, or the neuron and/or glial cell of the same patient before transfection. In some embodiments, the transfected neuron and/or glial cell demonstrates a reduction in the production of Lewy bodies between about 1% to about 300% or more compared to that of a neuron and/or glial cell in a PARK2-deficient patient, or the neuron and/or glial cell of the same patient before transfection. In some embodiments, production of Lewy bodies in the transfected neuron and/or glial cell decreases by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 90%, about 95%, about 99%, about 100%, about 200%, about 300% or more compared to that of a neuron and/or glial cell in a PARK2-deficient patient, or the neuron and/or glial cell of the same patient before transfection. In some embodiments, production of Lewy bodies in the transfected neuron and/or glial cell decreases compared to that of a neuron and/or glial cell in a PARK2-deficient patient, or the neuron and/or glial cell of the same patient before transfection for about 1 week to about 10 years or more. In some embodiments, production of Lewy bodies in the transfected neuron and/or glial cell decreases compared to that of a neuron and/or glial cell in a PARK2-deficient patient, or the neuron and/or glial cell of the same patient before transfection for about 1 week, about 1 month, about 2 months, about 3 months, about 4 months, about 5 months, about 6 months, about 8 months, about 1 year, about 2 years, about 5 years, and/or about 10 years or more. In some embodiments, production of Lewy bodies in the transfected neuron and/or glial cell decreases by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 90%, about 95%, about 99%, about 100%, about 200%, about 300% or more compared to that of a neuron and/or glial cell in a PARK2-deficient patient, or the neuron and/or glial cell of the same patient before transfection for about 1 week, about 1 month, about 2 months, about 3 months, about 4 months, about 5 months, about 6 months, about 8 months, about 1 year, about 2 years, about 5 years, and/or about 10 years or more.

In some embodiments, after transfection with a gene therapy construct of the instant disclosure, the neuron and/or glial cell expressing a parkin polypeptide or functional variant thereof as disclosed herein expresses a reduced amount of alpha-synuclein compared to that of a neuron and/or glial cell in a PARK2-deficient patient, or the neuron and/or glial cell of the same patient before transfection. In some embodiments, expression of alpha-synuclein in the transfected neuron and/or glial cell decreases between about 1% to about 300% or more compared to that of a neuron and/or glial cell in a PARK2-deficient patient, or the neuron and/or glial cell of the same patient before transfection. In some embodiments, expression of alpha-synuclein in the transfected neuron and/or glial cell decreases by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 90%, about 95%, about 99%, about 100%, about 200%, about 300% or more compared to that of a neuron and/or glial cell in a PARK2-deficient patient, or the neuron and/or glial cell of the same patient before transfection. In some embodiments, expression of alpha-synuclein in the transfected neuron and/or glial cell decreases compared to that of a neuron and/or glial cell in a PARK2-deficient patient, or the neuron and/or glial cell of the same patient before transfection for about 1 week to about 10 years or more. In some embodiments, expression of alpha-synuclein in the transfected neuron and/or glial cell decreases compared to that of a neuron and/or glial cell in a PARK2-deficient patient, or the neuron and/or glial cell of the same patient before transfection for about 1 week, about 1 month, about 2 months, about 3 months, about 4 months, about 5 months, about 6 months, about 8 months, about 1 year, about 2 years, about 5 years, and/or about 10 years or more. In some embodiments, the expression of alpha-synuclein in the transfected neuron and/or glial cell decreases by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 90%, about 95%, about 99%, about 100%, about 200%, about 300% or more compared to that of a neuron and/or glial cell in a PARK2-deficient patient, or the neuron and/or glial cell of the same patient before transfection for about 1 week, about 1 month, about 2 months, about 3 months, about 4 months, about 5 months, about 6 months, about 8 months, about 1 year, about 2 years, about 5 years, and/or about 10 years or more.

In some embodiments, after transfection with a gene therapy construct of the instant disclosure, the transfected neuron and/or glial cell expressing a parkin polypeptide or functional variant thereof as disclosed herein expresses a reduced amount of monoamine oxidases compared to that of a neuron and/or glial cell in a PARK2-deficient patient, or the neuron and/or glial cell of the same patient before transfection. In some embodiments, expression of monoamine oxidases in the transfected neuron and/or glial cell decreases between about 1% to about 300% or more compared to that of a neuron and/or glial cell in a PARK2-deficient patient, or the neuron and/or glial cell of the same patient before transfection. In some embodiments, expression of monoamine oxidases decreases by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 90%, about 95%, about 99%, about 100%, about 200%, about 300% or more compared to that of a neuron and/or glial cell in a PARK2-deficient patient, or the neuron and/or glial cell of the same patient before transfection. In some embodiments, expression of monoamine oxidases in the transfected neuron and/or glial cell decreases compared to that of a neuron and/or glial cell in a PARK2-deficient patient, or the neuron and/or glial cell of the same patient before transfection for about 1 week to about 10 years or more. In some embodiments, expression of monoamine oxidases in the transfected neuron and/or glial cell decreases compared to that of a neuron and/or glial cell in a PARK2-deficient patient, or the neuron and/or glial cell of the same patient before transfection for about 1 week, about 1 month, about 2 months, about 3 months, about 4 months, about 5 months, about 6 months, about 8 months, about 1 year, about 2 years, about 5 years, and/or about 10 years or more. In some embodiments, the expression of monoamine oxidases in the transfected neuron and/or glial cell decreases by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 90%, about 95%, about 99%, about 100%, about 200%, about 300% or more compared to that of a neuron and/or glial cell in a PARK2-deficient patient, or the neuron and/or glial cell of the same patient before transfection for about 1 week, about 1 month, about 2 months, about 3 months, about 4 months, about 5 months, about 6 months, about 8 months, about 1 year, about 2 years, about 5 years, and/or about 10 years or more.

In some embodiments, after transfection with a gene therapy construct of the instant disclosure, the transfected neuron and/or glial cell produces and/or releases an increased amount of dopamine compared to that of a neuron and/or glial cell in a PARK2-deficient patient, or the neuron and/or glial cell of the same patient before transfection. In some embodiments, the transfected neuron and/or glial cell produces and/or releases an increased amount of dopamine due to increased Tyrosine Hydroxylase (TH) levels due to expression of PARK-2 in the transfected neuron and/or glial cell. In some embodiments, production of dopamine in the transfected neuron and/or glial cell increases between about 1% to about 300% or more compared to that of a neuron and/or glial cell in a PARK2-deficient patient, or the neuron and/or glial cell of the same patient before transfection. In some embodiments, production of dopamine in the transfected neuron and/or glial cell increases by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 90%, about 95%, about 99%, about 100%, about 200%, about 300% or more compared to that of a neuron and/or glial cell in a PARK2-deficient patient, or the neuron and/or glial cell of the same patient before transfection. In some embodiments, production of dopamine in the transfected neuron and/or glial cell increases compared to that of a neuron and/or glial cell in a PARK2-deficient patient, or the neuron and/or glial cell of the same patient before transfection for about 1 week to about 10 years or more. In some embodiments, production of dopamine in the transfected neuron and/or glial cell increases in the transfected neuron and/or glial cell compared to that of a neuron and/or glial cell in a PARK2-deficient patient, or the neuron and/or glial cell of the same patient before transfection for about 1 week, about 1 month, about 2 months, about 3 months, about 4 months, about 5 months, about 6 months, about 8 months, about 1 year, about 2 years, about 5 years, and/or about 10 years or more. In some embodiments, production of dopamine in the transfected neuron and/or glial cell increases by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 90%, about 95%, about 99%, about 100%, about 200%, about 300% or more compared to that of a neuron and/or glial cell in a PARK2-deficient patient, or the neuron and/or glial cell of the same patient before transfection for about 1 week, about 1 month, about 2 months, about 3 months, about 4 months, about 5 months, about 6 months, about 8 months, about 1 year, about 2 years, about 5 years, and/or about 10 years or more.

In some embodiments, after transfection with a gene therapy construct of the instant disclosure, the transfected neuron and/or glial cell produces an increased amount of Tyrosine Hydroxylase (TH) compared to that of a neuron and/or glial cell in a PARK2-deficient patient, or the neuron of the same patient before transfection. See for example, Manfredsson et al. (2007), the contents of which are incorporated herein in their entireties for all purposes. In some embodiments, production of TH in the transfected neuron and/or glial cell increases between about 1% to about 300% or more compared to that of a neuron and/or glial cell in a PARK2-deficient patient, or the neuron and/or glial cell of the same patient before transfection. In some embodiments, production of TH in the transfected neuron increases by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 90%, about 95%, about 99%, about 100%, about 200%, about 300% or more compared to that of a neuron and/or glial cell in a PARK2-deficient patient, or the neuron and/or glial cell of the same patient before transfection. In some embodiments, production of TH in the transfected neuron and/or glial cell increases compared to that of a neuron and/or glial cell in a PARK2-deficient patient, or the neuron and/or glial cell of the same patient before transfection for about 1 week to about 10 years or more. In some embodiments, production of TH in the transfected neuron and/or glial cell increases in the transfected neuron compared to that of a neuron and/or glial cell in a PARK2-deficient patient, or the neuron and/or glial cell of the same patient before transfection for about 1 week, about 1 month, about 2 months, about 3 months, about 4 months, about 5 months, about 6 months, about 8 months, about 1 year, about 2 years, about 5 years, and/or about 10 years or more. In some embodiments, production of TH in the transfected neuron and/or glial cell increases by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 90%, about 95%, about 99%, about 100%, about 200%, about 300% or more compared to that of a neuron and/or glial cell in a PARK2-deficient patient, or the neuron and/or glial cell of the same patient before transfection for about 1 week, about 1 month, about 2 months, about 3 months, about 4 months, about 5 months, about 6 months, about 8 months, about 1 year, about 2 years, about 5 years, and/or about 10 years or more

In some embodiments, after transfection with a gene therapy construct of the instant disclosure, the transfected neuron and/or glial cell demonstrates increased autophagy compared to that of a neuron and/or glial cell in a PARK2-deficient patient, or the neuron and/or glial cell of the same patient before transfection. In some embodiments, autophagy in the transfected neuron and/or glial cell increases between about 1% to about 300% or more compared to that of a neuron and/or glial cell in a PARK2-deficient patient, or the neuron and/or glial cell of the same patient before transfection. In some embodiments, autophagy in the transfected neuron and/or glial cell increases by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 90%, about 95%, about 99%, about 100%, about 200%, about 300% or more compared to that of a neuron and/or glial cell in a PARK2-deficient patient, or the neuron and/or glial cell of the same patient before transfection. In some embodiments, autophagy in the transfected neuron and/or glial cell increases compared to that of a neuron and/or glial cell in a PARK2-deficient patient, or the neuron and/or glial cell of the same patient before transfection for about 1 week to about 10 years or more. In some embodiments, autophagy in the transfected neuron and/or glial cell increases in the transfected neuron compared to that of a neuron and/or glial cell in a PARK2-deficient patient, or the neuron and/or glial cell of the same patient before transfection for about 1 week, about 1 month, about 2 months, about 3 months, about 4 months, about 5 months, about 6 months, about 8 months, about 1 year, about 2 years, about 5 years, and/or about 10 years or more. In some embodiments, autophagy in the transfected neuron increases by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 90%, about 95%, about 99%, about 100%, about 200%, about 300% or more compared to that of a neuron and/or glial cell in a PARK2-deficient patient, or the neuron and/or glial cell of the same patient before transfection for about 1 week, about 1 month, about 2 months, about 3 months, about 4 months, about 5 months, about 6 months, about 8 months, about 1 year, about 2 years, about 5 years, and/or about 10 years or more.

In some aspects the constructs of the present disclosure are administered to a patient displaying symptoms of and/or diagnosed with a central nervous system disease or disorder. In some aspects the constructs of the present disclosure are administered to a patient at risk of developing symptoms of and/or being diagnosed with a central nervous system disease or disorder. In some embodiments, the central nervous system disease or disorder is Parkinson's Disease. In some embodiments, the Parkinson's Disease is an early-onset Parkinson's Disease. In some embodiments, the patient bears a mutation or deletion in PARK2 gene. In some embodiments, the patient bears a heterozygous mutation or deletion in the PARK2 gene. In some embodiments, the patient bears a homozygous mutation or deletion in the PARK2 gene. In some embodiments, the mutation in the PARK2 gene results in a loss of function or a decrease in function of the expressed parkin polypeptide. In some embodiments, the mutation is an exon mutation, an exon deletion, stop codon, an intron mutation, an intron deletion, a splice variant, a point mutation, a frameshift mutation, a deletion mutation, a disrupter mutation, a mutation that decreases copy number, and/or a mutation that increases protein turnover.

In some embodiments, the patient bears a non-PARK2 mutation associated with the development of Parkinson's Disease. In some embodiments, the patient bears a mutation in one or more of the SNCA, PARK7, PINK1, and/or LRK2 genes. In some embodiments, the patient bears a mutation in PARK2 and in one or more of the SNCA, PARK7, PINK1, and/or LRK2 genes. In some embodiments, the mutations in the SNCA, PARK7, PINK1, and/or LRK2 genes are loss of function mutations. In some embodiments, the patient is heterozygous for the mutation in one or more of the SNCA, PARK7, PINK1, and/or LRK2 genes. In some embodiments, the patient is homozygous for the mutation in one or more of the SNCA, PARK7, PINK1, and/or LRK2 genes. In some embodiments, the mutation in one or more of the SNCA, PARK7, PINK1, and/or LRK2 genes is an exon mutation, an exon deletion, stop codon, an intron mutation, an intron deletion, a splice variant, a point mutation, a frameshift mutation, a deletion mutation, a disrupter mutation, a mutation that decreases copy number, and/or a mutation that increases protein turnover.

In some embodiments, administration of the pharmaceutical compositions and constructs of the present disclosure replaces one PARK2 gene in the patient's cell. In some embodiments, administration of the pharmaceutical compositions and constructs of the present disclosure replaces both PARK2 genes in the patient's cell. In some embodiments, administration of the pharmaceutical compositions and constructs of the present disclosure adds additional copies of the PARK2 gene in the patient's cell. In some embodiments, administration of the pharmaceutical compositions and constructs of the present disclosure adds between 1 and 10 additional PARK2 genes in the patient's cell. In some embodiments, administration of the pharmaceutical compositions and constructs of the present disclosure adds about 1, about 2, about 3, about 4, about 5, about 6, about 7, about 8, about 9, or about 10 additional PARK2 genes in the patient's cell.

In some aspects, administration of a construct of the present disclosure to a patient treats, improves, prevents, ameliorates, and/or delays one or more symptoms of Parkinson's Disease. In some embodiments, the one or more symptoms of Parkinson's Disease includes, but is not limited to, motor deficits, tremors, bradykinesia (slowed movement), rigid muscles, impaired posture and balance, loss of automatic movements, speech changes, writing changes, depression, swallowing problems, decreased cardiac function, sleep disorders, dementia, cognitive problems, emotional changes (e.g. fear, anxiety, or loss of motivation), blood pressure changes, fatigue, pain, involuntary movements, shuffling gait, dizziness, amnesia, confusion, voice box spasms, distorted sense of smell, jaw stiffness or reduced facial expression, and weight loss.

In some embodiments, after transfection with a gene therapy construct of the instant disclosure, treated patient demonstrates reduction in one or more symptoms of Parkinson's Disease compared to an untreated Parkinson's Disease patient or the same patient before treatment. In some embodiments, one or more symptoms of Parkinson's Disease are reduced between about 1% to about 300% or more compared to an untreated Parkinson's Disease patient or the same patient before treatment. In some embodiments, one or more symptoms of Parkinson's Disease is reduced by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 90%, about 95%, about 99%, about 100%, about 200%, about 300% or more compared to an untreated Parkinson's Disease patient or the same patient before treatment. In some embodiments, one or more symptoms of Parkinson's Disease are decreased compared to compared to an untreated Parkinson's Disease patient or the same patient before treatment for about 1 week to about 10 years or more. In some embodiments, one or more symptoms of Parkinson's Disease are decreased compared to an untreated Parkinson's Disease patient or the same patient before treatment for about 1 week, about 1 month, about 2 months, about 3 months, about 4 months, about 5 months, about 6 months, about 8 months, about 1 year, about 2 years, about 5 years, and/or about 10 years or more. In some embodiments, one or more symptoms of Parkinson's Disease is decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 90%, about 95%, about 99%, about 100%, about 200%, about 300% or more compared to an untreated Parkinson's Disease patient or the same patient before treatment for about 1 week, about 1 month, about 2 months, about 3 months, about 4 months, about 5 months, about 6 months, about 8 months, about 1 year, about 2 years, about 5 years, and/or about 10 years or more.

In some embodiments, administration of a gene therapy construct of the present disclosure increases the number of dopaminergic neurons in the treated patient compared to an untreated Parkinson's Disease patient or the same patient before treatment. In some embodiments, the number of dopaminergic neurons in the treated patient increases between about 1% to about 300% or more compared to an untreated Parkinson's Disease patient or the same patient before treatment. In some embodiments, the number of dopaminergic neurons increases by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 90%, about 95%, about 99%, about 100%, about 200%, about 300% or more in a treated patient compared to an untreated Parkinson's Disease patient or the same patient before treatment. In some embodiments, the number of dopaminergic neurons increases compared to an untreated Parkinson's Disease patient or the same patient before treatment for about 1 week to about 10 years or more. In some embodiments, the number of dopaminergic neurons increases in an untreated Parkinson's Disease patient or the same patient before treatment for about 1 week, about 1 month, about 2 months, about 3 months, about 4 months, about 5 months, about 6 months, about 8 months, about 1 year, about 2 years, about 5 years, and/or about 10 years or more. In some embodiments, the number of dopaminergic neurons increases by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 90%, about 95%, about 99%, about 100%, about 200%, about 300% or more compared to an untreated Parkinson's Disease patient or the same patient before treatment for about 1 week, about 1 month, about 2 months, about 3 months, about 4 months, about 5 months, about 6 months, about 8 months, about 1 year, about 2 years, about 5 years, and/or about 10 years or more.

In some embodiments, administration of a gene therapy construct of the present disclosure strengthens connections among dopaminergic neurons in the treated patient compared to an untreated Parkinson's Disease patient or the same patient before treatment. The strength or numbers of connections among dopaminergic neurons can be determined using the metabolic network analysis disclosed in Niethammer et al. (2018), the contents of which are incorporated herein in their entireties for all purposes. In some embodiments, the connection strength of dopaminergic neurons in the treated patient increases between about 1% to about 300% or more compared to an untreated Parkinson's Disease patient or the same patient before treatment. In some embodiments, the connection strength dopaminergic neurons increases by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 90%, about 95%, about 99%, about 100%, about 200%, about 300% or more in a treated patient compared to an untreated Parkinson's Disease patient or the same patient before treatment. In some embodiments, the connection strength of dopaminergic neurons increases compared to an untreated Parkinson's Disease patient or the same patient before treatment for about 1 week to about 10 years or more. In some embodiments, the connection strength of dopaminergic neurons increases in an untreated Parkinson's Disease patient or the same patient before treatment for about 1 week, about 1 month, about 2 months, about 3 months, about 4 months, about 5 months, about 6 months, about 8 months, about 1 year, about 2 years, about 5 years, and/or about 10 years or more. In some embodiments, the connection strength of dopaminergic neurons increases by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 90%, about 95%, about 99%, about 100%, about 200%, about 300% or more compared to an untreated Parkinson's Disease patient or the same patient before treatment for about 1 week, about 1 month, about 2 months, about 3 months, about 4 months, about 5 months, about 6 months, about 8 months, about 1 year, about 2 years, about 5 years, and/or about 10 years or more.

### Pharmaceutical Compositions & Methods of Administration

The present disclosure provides pharmaceutical compositions comprising an AAV capsid incorporating a gene therapy construct of the present disclosure and one or more diluents, preservatives, stabilizers, carriers, and/or pharmaceutical excipients. In some embodiments, the preservatives include chlorobutanol, potassium sorbate, sorbic acid, sulfur dioxide, propyl gallate, the parabens, ethyl vanillin, glycerin, phenol, and parachlorophenol. In some embodiments, the stabilizers include gelatin and albumin. In some embodiments, the pharmaceutical composition is free of DNA and/or other cellular material.

The present gene therapy constructs are administered in sufficient amounts to transfect the cells and to provide sufficient levels of gene transfer and expression to provide a therapeutic benefit without undue adverse effects, or with medically acceptable physiological effects, which can be determined by those skilled in the medical arts.

The gene therapy constructs of the present disclosure may be administered using any appropriate route. In some embodiments, the gene therapy constructs of the present disclosure are administered directly to the brain. In some embodiments, the administration to the brain is local administration. In some embodiments, the gene therapy constructs of the present disclosure are administered to the substantia nigra.

In some embodiments, the gene therapy constructs of the present disclosure are administered to the cells in the brain that produce tyrosine hydroxylase (TH).

In some embodiments, local administration to the brain requires craniotomy and injection of a gene therapy construct of the present disclosure. In some embodiments, administration includes, but is not limited to, direct injection, injection into the cerebrospinal fluid (CSF), intrathecal injection, intravascular administration, cerebral, intrascisternal, inraputaminal, Intranigral, intrahippocampal, intrastiatal, intracerebroventricular, and intramuscular injection targeting motor and sensory neurons.

In some embodiments, the gene therapy construct injected is the naked polynucleotide. In some embodiments, the gene therapy construct injected is associated with one or more molecules (e.g. lipids, micelles, cationic polymers, etc.). In some embodiments, the gene therapy construct is associated with one or more proteins. In some embodiments, the one or more proteins includes a ferrying protein. In some embodiments, the one or more proteins includes a viral protein. In some embodiments, the viral protein is a capsid protein. In some embodiments, the gene therapy construct is packaged into a viral vector. In some embodiments, the viral vector is a herpes simplex vector, a lentiviral vector, an adeno-associated viral vector, or an adeno-associated vector. In some embodiments, the adeno-associated vector is AAV5.

In some embodiments, 1×10⁶-1×10¹⁴ vector genomes per kilogram body mass of the subject (vg/kg) of the gene therapy construct of the present disclosure are administered to the subject. In some embodiments, 1×10⁶-1×10¹⁴ vector genomes per kilogram body mass of the subject (vg/kg) of the gene therapy construct of the present disclosure are injected to the subject's brain. In some embodiments, 1×10⁶-1×10¹⁴ vector genomes per kilogram body mass of the subject (vg/kg) of the gene therapy construct of the present disclosure are administered to the subject's CSF. In some embodiments, 1×10⁷-1×10⁸ vector genomes per kilogram body mass of the subject (vg/kg) of the gene therapy construct of the present disclosure are administered to the subject. In some embodiments, 1×10⁷-1×10⁹ vector genomes per kilogram body mass of the subject (vg/kg) of the gene therapy construct of the present disclosure are administered to the subject. In some embodiments, 1×10⁶-1×10¹⁴, 1×10⁷-1×10¹³, 1×10⁸-1×10¹², 1×10⁹-1×10¹¹, 1×10¹⁰-1×10¹¹, 1×10¹¹-1×10¹², 1×10¹²-1×10¹³, 1×10¹³-1×10¹⁴ vector genomes per kilogram body mass of the subject (vg/kg) of the gene therapy construct of the present disclosure are administered to the subject.

In some embodiments, 1×10⁶-1×10¹⁴ gc/mL of the gene therapy construct of the present disclosure are administered to the subject. In some embodiments, 1×10⁶-1×10¹⁴ gc/mL of the gene therapy construct of the present disclosure are injected to the subject's brain. In some embodiments, 1×10⁶-1×10¹⁴ gc/mL of the gene therapy construct of the present disclosure are administered to the subject's CSF. In some embodiments, 1×10⁷-1×10⁸ gc/mL of the gene therapy construct of the present disclosure are administered to the subject. In some embodiments, 1×10⁷-1×10⁹ gc/mL of the gene therapy construct of the present disclosure are administered to the subject. In some embodiments, 1×10⁶-1×10¹⁴, 1×10⁷-1×10¹³, 1×10⁸-1×10¹², 1×10⁹-1×10¹¹, 1×10¹⁰-1×10¹¹, 1×10¹¹-1×10¹², 1×10¹²-1×10¹³, 1×10¹³ gc/mL of the gene therapy construct of the present disclosure are administered to the subject. In some embodiments, 1×10¹³ gc/mL of the gene therapy construct of the present disclosure are administered to the subject.

In some embodiments, the dose administered to the patient is informed by the type of genetic mutation the patient bears. In some embodiments, patients bearing a heterozygous mutation (e.g. patients with one wild type PARK2 gene) will be administered a lower dose of the gene therapy construct (e.g. less than 1×10¹³ gc/mL). In some embodiments, patient bearing a homozygous mutation (e.g. patients with no wild type PARK2 gene) will be administered a higher dose (e.g. 1×10¹³ gc/mL or higher). In some embodiments, patients bearing a mutation in a non-PARK2 gene (e.g. one or more of the SNCA, PARK7, PINK1, and/or LRRK2 genes) will be administered a lower dose (e.g. less than 1×10¹³ gc/mL).

The dose administered to a patient may also be informed by the age of the patient (e.g. pediatric, adolescent, adult) and severity of the disease. In some embodiments, a pediatric patient will be administered a lower dose than that administered to an adolescent or an adult. In some embodiments, an adolescent patient will be administered a lower dose than that administered to an adult. In some embodiments, patients displaying more severe symptoms of the disease will be administered a greater dose than patients with more mild symptoms, or patients in the early stage of the disease. In some embodiments, patients at risk of developing a disease (e.g. those bearing one or more PARK2 mutations) will be administered a lower dose than that administered to patients displaying symptoms.

The pharmaceutical compositions may be administered to a patient as many times as appropriate. In some embodiments, the pharmaceutical composition is administered to the patient daily, weekly, monthly, or yearly. In some embodiments, the pharmaceutical composition is administered between 1 and 50 times. In some embodiments, the pharmaceutical composition is administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 times. In some embodiments, the dose of the pharmaceutical composition remains the same for one or more administrations. In some embodiments, the dose of the pharmaceutical composition remains the same for each administration. In some embodiments, the dose of the pharmaceutical composition changes for one or more administrations.

As taught herein, variant parkin polypeptides may contain activating mutations that result in increased polypeptide activity compared to wild type parkin. In these cases, lower levels of expression of the activated parkin variant may demonstrate activity at wild type levels or near wild type levels. Thus, in some embodiments, administration of lower doses of the activated parkin variant demonstrates an effective treatment while also demonstrating fewer deleterious side effects associated with AAV vector delivery (e.g. inflammation, edema, hemorrhage, etc.).

Unless defined otherwise, all technical and scientific terms herein have the same meaning as commonly understood by one of ordinary skill in the art to which this disclosure belongs. Although any methods and materials, similar or equivalent to those described herein, can be used in the practice or testing of the present disclosure, the preferred methods and materials are described herein.

### Examples of Non-Limiting Embodiments of the Disclosure

Embodiments of the present subject matter disclosed herein may be beneficial alone or in combination with one or more other embodiments. Without limiting the foregoing description, certain non-limiting embodiments of the disclosure, numbered 1 to 65, are provided below. As will be apparent to those of skill in the art upon reading this disclosure, each of the individually numbered embodiments may be used or combined with any of the preceding or following individually numbered embodiments. This is intended to provide support for all such combinations of embodiments and is not limited to combinations of embodiments explicitly provided below.

Embodiment 1. A method of treating, preventing, or ameliorating a central nervous system disorder or a symptom thereof comprising administering a construct comprising a parkin (PARK2) gene to a patient in need thereof.

Embodiment 2. The method of embodiment 1, wherein the PARK2 gene is a mutant PARK2 gene.

Embodiment 3. The method of embodiment 1 or 2, wherein the PARK2 gene is a mutant PARK2 gene that encodes for a protein that increases activity of parkin.

Embodiment 4. The method of any of embodiments 1-3, wherein the activating mutant PARK2 gene codes for a protein displaying increased auto-ubiquitination compared to the wild type PARK2 gene.

Embodiment 5. The method of any of embodiments 1-4, wherein the activating mutant PARK2 gene comprises the nucleic acid sequence of SEQ ID NO; 3, 5, or 7.

Embodiment 6. The method of any one of embodiments 1-5, wherein the construct is encased in a viral vector or portion thereof.

Embodiment 7. The method of any of embodiments 1-6, wherein the viral vector is an adeno-associated viral vector or portion thereof.

Embodiment 8. The method of any of embodiments 1-7, wherein the adeno-associated viral vector or portion thereof is AAV1, AAV2, AAV3, AAV4, AAV5, AAV6, AAV7, AAV8, AAV9, AAV10, AAV11, AAV12, or AAV13.

Embodiment 9. The method of any of embodiments 1-8, wherein the adeno-associated viral vector of portion thereof is AAV9.

Embodiment 10. The method of any one of embodiments 1-9, wherein the PARK2 gene is under control of a tissue specific promoter.

Embodiment 11. The method of any of embodiments 1-10, wherein the PARK2 gene is under control of a neuron-specific promoter.

Embodiment 12. The method of any of embodiments 1-11, wherein the PARK2 gene is under control of a ubiquitous promoter.

Embodiment 13. The method of any one of embodiments 1-12, wherein the PARK2 gene is under control of a promoter selected from the list: chicken-beta-actin (CBA), human beta actin (HuBa), cytomegalovirus (cMV), CAG, PGL, EF1-alpha, GAPDFI, SV40, FIBV, human synapsin (hSYN1), alpha-internexin (INA), nestin (NES), tyrosine hydroxylase (TH), forkhead box A2 (FOXA2), calmodulin-dependent protein kinase II (CAMKII), and neuron-specific enolase (NSE).

Embodiment 14. The method of any one of embodiments 1-13, wherein the construct comprises an enhancer.

Embodiment 15. The method of any of embodiments 1-14, wherein the enhancer is a CMV enhancer.

Embodiment 16. The method of any of embodiments 1-15, wherein the construct comprises an AAV inverted terminal repeat (ITR).

Embodiment 17. The method of any of embodiments 1-16, wherein the construct comprises two AAV inverted terminal repeats (ITRs) flanking the expression cassette.

Embodiment 18. The method of any one of embodiments 1-17, wherein the construct is administered directly into the brain.

Embodiment 19. The method of any one of embodiments 1-18, wherein the construct is administered by intrathecal administration.

Embodiment 20. The method of any one of embodiments 1-19, wherein the central nervous system disorder is Parkinson's Disease.

Embodiment 21. The method of any of embodiments 1-20, wherein the patient displays one or more symptoms of Parkinson's Disease.

Embodiment 22. The method of any of embodiments 1-20, wherein the patient is at risk of developing one or more symptoms of Parkinson's Disease.

Embodiment 23. The method of any of embodiments 1-22, wherein the one or more symptoms of Parkinson's Disease is selected from the group consisting of: motor deficits, tremors, bradykinesia (slowed movement), rigid muscles, impaired posture and balance, loss of automatic movements, speech changes, writing changes, depression, swallowing problems, decreased cardiac function, sleep disorders, dementia, cognitive problems, emotional changes (e.g. fear, anxiety, or loss of motivation), blood pressure changes, fatigue, pain, involuntary movements, shuffling gait, dizziness, amnesia, confusion, voice box spasms, distorted sense of smell, jaw stiffness or reduced facial expression, and weight loss.

Embodiment 24. The method of any of embodiments 1-23, wherein administration of the construct increases the number of dopaminergic neurons in the patient.

Embodiment 25. A recombinant gene therapy vector comprising a mutant parkin (PARK2) gene.

Embodiment 26. The vector of embodiment 25, wherein the PARK2 gene is a mutant PARK2 gene that encodes for a protein that increases activity of parkin.

Embodiment 27. The vector of any of embodiments 25-36, wherein the activating mutant PARK2 gene codes for a protein displaying increased auto-ubiquitination compared to the wild type PARK2 gene.

Embodiment 28. The vector of any of embodiments 25-27, wherein the mutant PARK2 gene codes for a polypeptide with a mutation at the amino acid position of 146, 183 and/or 463 of parkin.

Embodiment 29. The vector of any of embodiments 25-28, wherein the mutant PARK2 gene codes for a polypeptide with a mutation at the amino acid position of 146 and/or 183 of parkin.

Embodiment 30. The vector of any of embodiments 25-29, wherein the mutation of an amino acid is to a tyrosine residue.

Embodiment 31. The vector of any of embodiments 25-30, wherein the polypeptide comprises SEQ ID NO: 4, 6, or 8.

Embodiment 32. The vector of any of embodiments 25-31, wherein the polypeptide comprises SED ID NO: 4 or 6.

Embodiment 33. The vector of any of embodiments 25-32, wherein the construct is encased in a viral vector or portion thereof.

Embodiment 34. The vector of any of embodiments 25-33, wherein the viral vector is an adeno-associated viral vector or portion thereof.

Embodiment 35. The vector of any of embodiments 25-34, wherein the adeno-associated viral vector or portion thereof is AAV1, AAV2, AAV3, AAV4, AAV5, AAV6, AAV7, AAV8, AAV9, AAV10, AAV11, AAV12, or AAV13.

Embodiment 36. The vector of any of embodiments 25-35, wherein the adeno-associated viral vector of portion thereof is AAV9.

Embodiment 37. The vector of any of embodiments 25-36, wherein the PARK2 gene is under control of a tissue specific promoter.

Embodiment 38. The vector of any of embodiments 25-37, wherein the PARK2 gene is under control of a neuron-specific promoter.

Embodiment 39. The vector of any of embodiments 25-38, wherein the PARK2 gene is under control of a ubiquitous promoter.

Embodiment 40. The vector of any of embodiments 25-39, wherein the PARK2 gene is under control of a promoter selected from the list: chicken-beta-actin (CBA), human beta actin (HuBa), cytomegalovirus (cMV), CAG, PGL, EF1-alpha, GAPDFI, SV40, FIBV, human synapsin (hSYN1), alpha-internexin (INA), nestin (NES), tyrosine hydroxylase (TH), forkhead box A2 (FOXA2), calmodulin-dependent protein kinase II (CAMKII), and neuron-specific enolase (NSE).

Embodiment 41. The vector of any of embodiments 25-40, wherein the construct comprises an enhancer.

Embodiment 42. The vector of claim 41, wherein the enhancer is a CMV enhancer.

Embodiment 43. The vector of any of embodiments 25-42, wherein the construct comprises an AAV inverted terminal repeat (ITR).

Embodiment 44. The vector of any of embodiments 25-43, wherein the construct comprises two AAV inverted terminal repeats (ITRs) flanking the expression cassette.

Embodiment 45. An isolated polypeptide comprising SEQ ID NO: 4 or 6.

This disclosure is further illustrated by the following non-limiting examples.

### EXAMPLES

### Example 1—Design of Gene Therapy Constructs for AAV9 Delivery

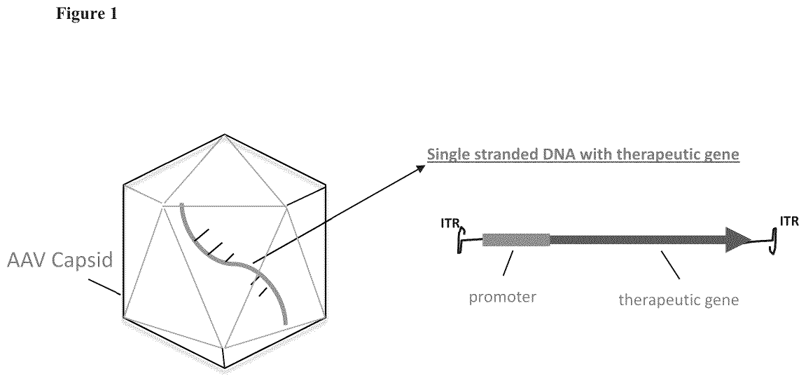

The overall design of the gene therapy constructs used in the present disclosure is shown in FIG. 1 The PARK2 gene is represented as the cDNA human sequence, and several variants were tested: W183Y, F208Y, and F463 Y along with a wild type PARK2. The expression of these cDNA sequences was controlled by a CBA promoter for maximum expression. The single-stranded DNA containing the PARK2 cDNA sequence is flanked by inverted terminal repeats (ITR) for packaging into an AAV9 capsid for intrathecal deliver to parenchymal tissue in the brain. The spread of the virus and the expression of the PARK2 transgene will be measured.

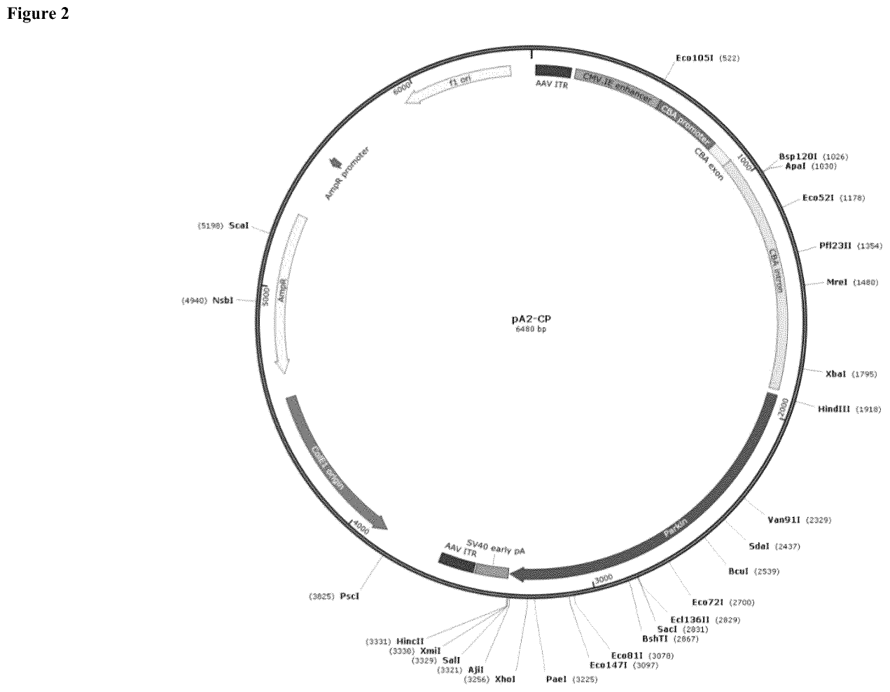

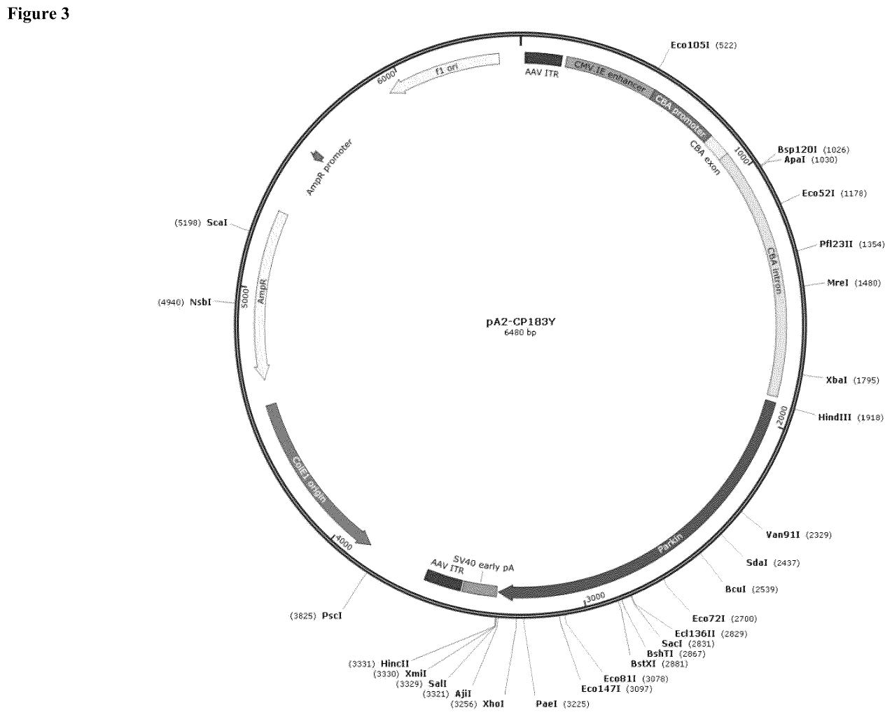

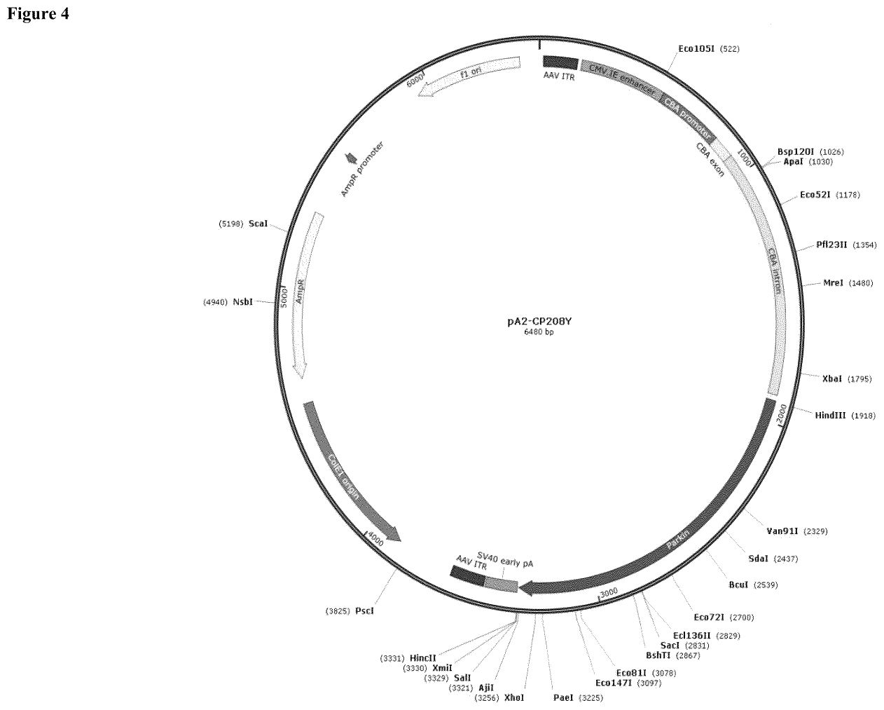

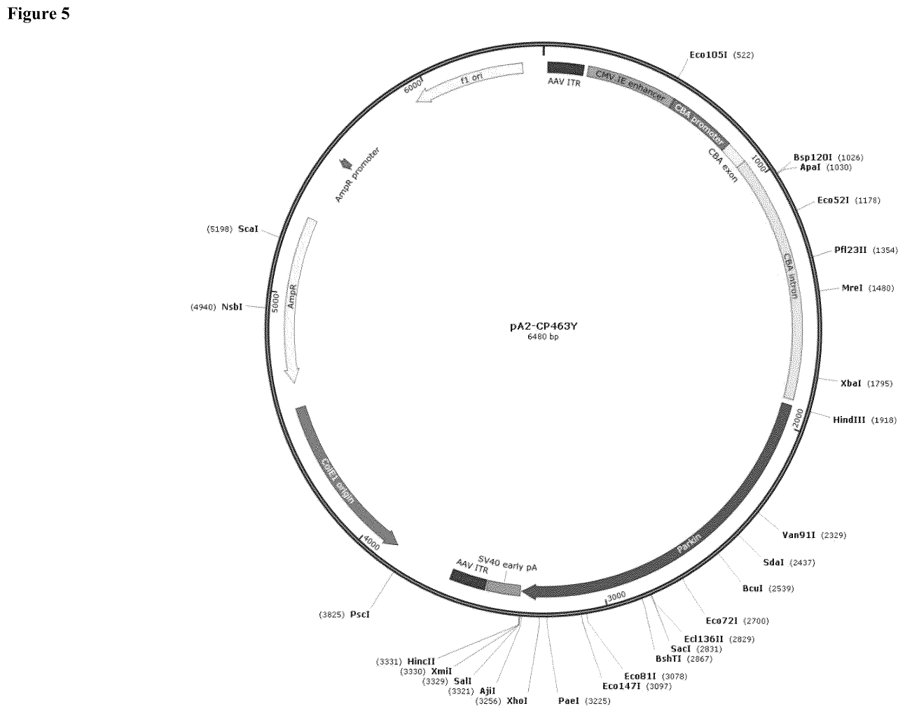

FIGS. 2-4 show the AAV9 constructs for gene therapy constructs containing the wild type parkin (PARK2), W183Y mutant, F208Y mutant, and F463Y mutants respectively.

### Example 2—PARK2 Mutants Increase the Activity of Parkin

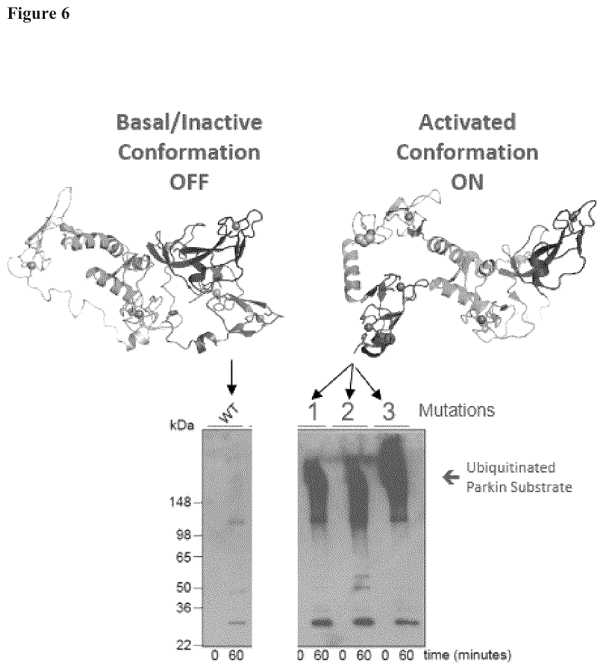

The parkin protein is in the “OFF” state until activated with stimuli. (FIG. 6). However, the W183Y, F208Y, and F463Y mutations change this state and are activating mutations that increase activity for the “ON” state.

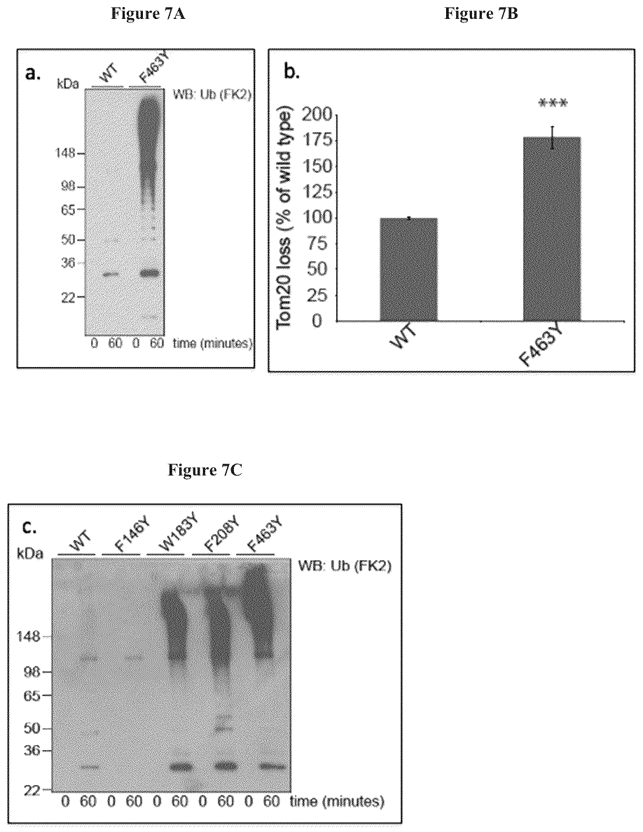

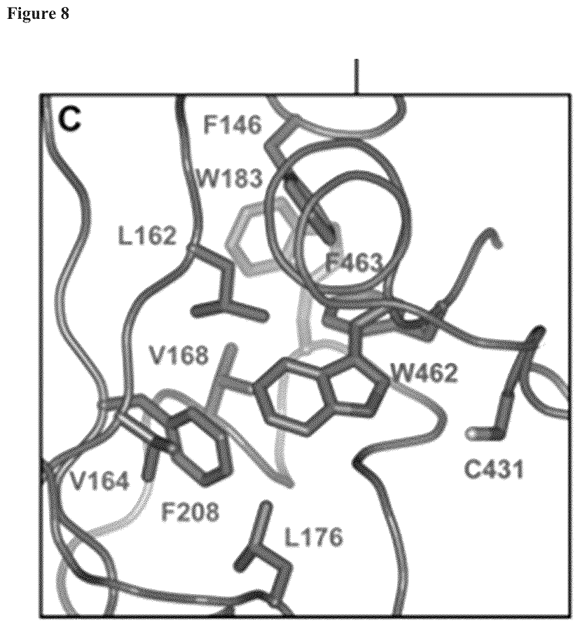

As shown in FIG. 7, the F463Y PARK2 mutant increases the activity of parkin in cells. This mutation is in the R2 residue (FIG. 7C), and contains a change from a phenylalanine (F) to tyrosine (Y) at residue 463. The crystal structure (FIG. 8) shows that the F463 residue is proximal to the active site cysteine of Parkin C431, and buried within the R0/R2 interface. Residues F146, W183, and F208 lie in the R0 region, and residues W462 and F463 lie in the R2 region.

An auto-ubiquitination assay was performed using the mutants and wild type parkin polypeptides. The principle of this assay is that the E3 Ligase Parkin catalyzes the transfer of Ubiquitin to target proteins, but also has the ability to auto-ubiquitinate. The phospho-Ubiquitin (pUb) is a purified cellular cofactor for Parkin and is added to the assay to decrease the energy barrier to activation of Parkin to auto-ubiquitinate though the E1-E2 cascade reactions. The Parkin sequences are tagged with a His marker, and the use of a Eu cryptate Ubiquitin and anti 6His-d2 that binds to the His tagged Parkin will give a signal when the Eu cryptate-Ubiquitin is auto-ubiquitinated onto the Parkin which can be monitored by TR-FRET. Mutations that increase parkin activation can be identified by an increase of the 0% activation (e.g. pUb) TR-FRET signal.

### Assay Conditions:

| Enzyme Reaction (15min pre-incubation with Parkin, pUb and activator only) | |
|---|---|
| Parkin: | 196 nM |
| pUb: | 196 nM |
| DMSO: | 1% DMSO |
| E1: | 5 nM |
| E2: | 50 nM |
| Ubiquitin Eu: | 8.8 nM |
| Reaction time: | 120 minutes |
| Temperature: | 22° C. |
| Total volume: | 10 μl reaction |

Reactions may be terminated by the addition of SDS-loading buffer. Western Blot detection was performed with Anti-ubiquitin Ab (FK2) with Ab used at 1:1000 dilution.
Reaction also be detected by taking 10 μl of Enzyme Reaction above and add 10 μl detection Reagent Z under the following conditions:

| | |
|---|---|
| Reaction time: | 60 minutes |
| Temperature: | 22° C. |
| Total volume: | 20 μl |

Data Analysis: The Data was read in CSV files. There are two tables in those CSV files, which are the values of 655 nm (channel 1) and 615 nm (channel 2) wavelengths respectively. The data was converted to an HTRF Ratio=(Channel 1/Channel 2)*10,000

The average of all the 0 uM controls (DMSO only)=BKGD (Background-0% activation). Subtract BKGD from each HTRF Ratio value=HTRF-BKGD. The average of all the 100 uM control activator in DMSO controls=Max (100% activation). The following equation is then used to calculate % Activation for each well/candidate as follows: % Activation=(HTRF−BKGD/Max)*100.

The % Activation of compound titration is then used to find activation EC50 or highest % activation if less than 75% activation is seen for the candidate compound.

XLFIT5 model 205 was applied for the data analysis. EC50 fit model (4 Parameter Logistic Model/Sigmoidal dose-Response Model); fit=(A+((B−A)/(1+((C/x)−D)))); res=(y-fit). The parameters are:

| | |
|---|---|
| A: | Bottom |
| B: | Top |
| C: | Relative EC50 |
| D: | Hill Slope |

### Constrains set to Bottom=0 and Top=100.

FIGS. 7A & C show that the W183Y, F208Y, and F463Y mutant purified proteins demonstrate increased autoubiquitination.

### Tomm20 Assay

The Tomm20 loss assay screens for the ability for cells to augment mitophagy, the process of removing damaged mitochondria which is compromised in Parkinson's Disease patients and in animal and cellular model systems for PD. Here, HeLa cells were transfected with Parkin cDNA to assess Parkin-dependent induction of mitophagy. Cells were transfected with a protein comprising one of: wild type Parkin; the W183Y parkin mutant; the F208Y parkin mutant; or the F463Y parkin mutant. 4000 cells of each transformant were seeded in each well of a 96 well plate (Parkin Elmer ViewPlate-96 F TC, cat. N. 6005182) and left to grow for 24 hours. HeLa cells were chosen for these experiments as HeLa cells do not express any endogenous Parkin protein due to genetic deletion.

Subsequently cells were incubated with vehicle (DMSO) or 6 μM CCCP for each condition run in replicate of five. After 20 hours cells were processed for immunofluorescence.

Immunofluorescence: Samples were fixed in 4% PFA for 25 minutes RT and permeabilized with PBS 0.1% Triton-X100 for 3 minutes on ice, blocked with PBS 3% BSA, 0.3% Triton-X100 for 2 hours RT, followed by overnight incubation with primary antibody at 4° C. (0.5 μg/ml rabbit Tomm20 antibody FL-145; Santa Cruz Biotechnology) diluted in PBS 0.1% BSA, 0.3% Triton-X100. The secondary goat anti-rabbit antibody conjugated with DyLight 649 (Jackson ImmunoResearch) was applied for 1 hour at room temperature at a concentration of 2.8 μg/ml in conjunction with 1 μg/ml Hoechst33342.

Cells were imaged using an Olympus ScanR automated microscope equipped with motorized stage and 20×APO planar objective. 18 images were acquired for each well using the following combination of excitation/emission filters: Hoechst33342 was excited through a 350/50 nm band pass filter and fluorescence intensity was collected through a 460/30 band pass filter. DyLight 649 was excited through a 640/30 nm band pass filter and fluorescence intensity was collected through 700/75 band pass filter. Images were processed and analyzed as described in the Image Analysis section.

Image analysis: Images were processed and analyzed using Columbus HCS Analysis software (Version 2.5.0., PerkinElmer) as follows:

Tomm20 fluorescence intensity was corrected using the parabola algorithm. Hoechst 33342 fluorescence was used to identify and count cells. Cells were segmented according to Tomm20 fluorescence intensity. Spot detection was optimized to recognize number and total cellular area of Tomm20 stained clusters (mitochondria).

Tomm20 staining intensity, spot numbers and spot area were used to train a linear classifier algorithm that discriminated between Tomm20 positive (high intensity, spot numbers and spot area) and Tomm20 negative cells (low intensity, spot numbers and spot area).

Bar graphs were generated reporting the number of Tomm20 negative cells expressed as percentage of total cells imaged for each well. Results were shown as mean±SD of a representative experiment performed in triplicate.). Results were shown as mean±SD of a representative experiment performed in triplicate. +++ indicates >70% effect at 10 μM; ++ indicates 69%-31% effect at 10 μM; + indicates <30% effect at 10 μM; NA=not available.

FIG. 7B shows that the expression of the F463Y parkin mutant exhibits increased functional mitophagy activity compared to wild type parkin as screened using the Tomm20 loss assay.

### Example 3—Production of AAV5 Constructs for Gene Therapy Delivery of Wild Type Parkin and Mutants

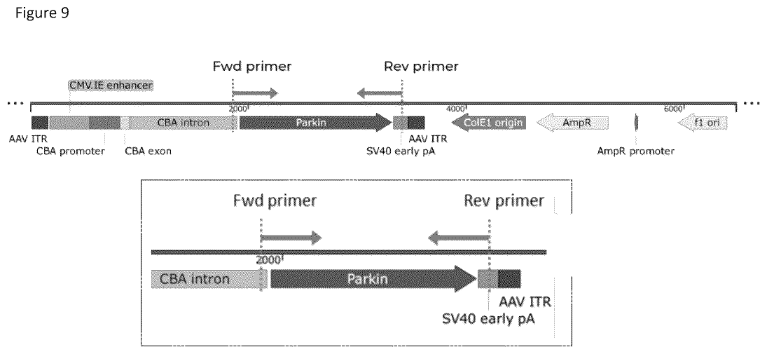

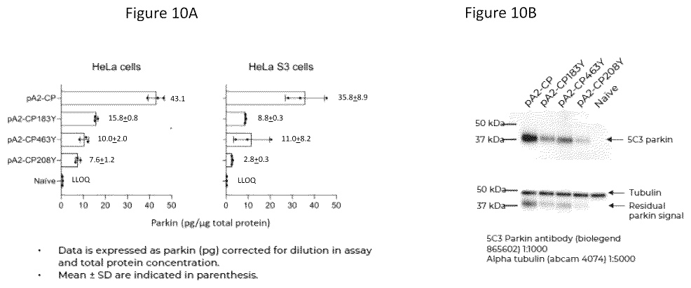

FIG. 9 shows plasmids used encode the recombinant AAV genome (AAV2 ITRs, chicken beta-actin promoter including a CMV enhancer element (CBA), transcript variant 1 of the human wild type Parkin gene or activating variants of the Parkin gene with single amino acid mutations at position W183Y, F208Y and F463Y, and the early poly A from SV40), essential adenoviral helper genes, AAV2 Rep and AAV5 Cap genes (Grimm et al 1998). The expression of the cDNA was controlled by a CBA promoter for maximum expression. The insertion of the Parkin gene was verified by amplifying the Parkin gene using primers outside of the Parkin sequence.

To detect expression levels, each of the vectors was transfected into HeLa and HeLa S3 cells. (FIG. 10)

HeLa S3 cells were grown in ten 175 cm² flasks and were co-transfected at a confluency of 70-80% using the calcium-phosphate precipitation method. Three days after transfection, cells were harvested using DPBS with 5 mM EDTA, resuspended in 30 ml buffer (50 mM Tris, 150 mM NaCl, pH 8.5) after centrifugation (1000 RCF for 5 minutes) and lysed by freezing in a bath of dry ice and ethanol. The lysate was thawed in a 37° C. water bath and treated with 11 units/ml Benzonase for 30 minutes. The preparation was clarified by centrifugation (3220 RCF for 25 minutes) and AAV purified using a discontinuous Iodixanol gradient, followed by anion exchange chromatography (Zolotukhin et al 1999) using a Mustang Q adsorption device (Pall). Buffer exchange to DPBS and concentration was done using a 100 kDa molecular weight cut-off centrifugation filter unit. Titers of the stock solution were determined by quantitative PCR using primers and hydrolysis probe targeting the ITR sequence (Aurnhammer et al 2012). Before being used in an experiment, vectors were diluted in DPBS and re-titered. Glass capillaries used for animal injections were pre-coated with the same AAV vector, before injection.

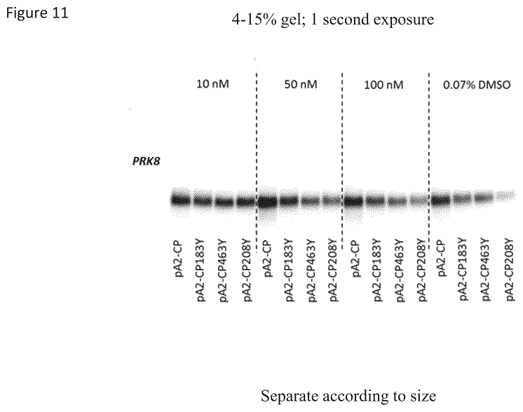

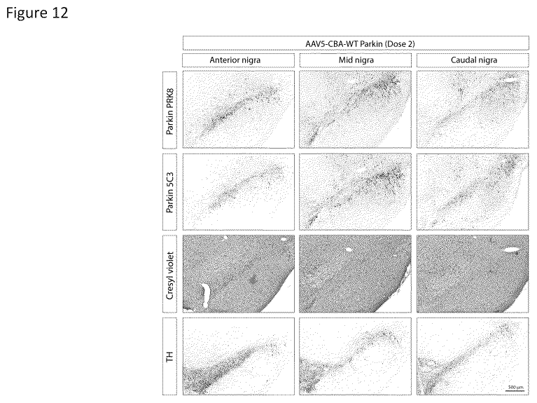

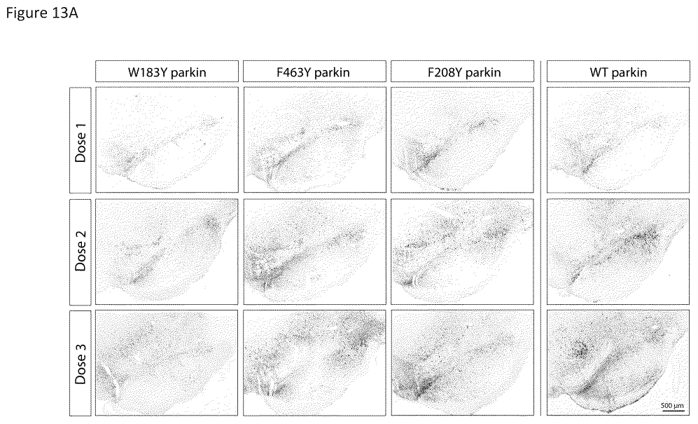

To confirm activity of the variant parkin polypeptides, HeLa S3 cells were transfected by seeding 0.8 million cells/mL in a 6 well plate, and after 24 hours, transfecting the cells with 2500 ng of DNA per well in medium containing 10% FBS. Cells were transfected with: pA2-CP (wt parkin); pA2-CP183Y; pA2-CP208Y; pA2-CP463Y; a naïve control, or a pA2-CG (GFP control for visualization). 6 hours after transfection, the medium was changed to 4% FBS, and the cells were incubated for 50 hours. On day 4, the cells were treated with 10, 50, or 100 nM the proteasome inhibitor epoxomicin or vehicle (0.07% DMSO). The cells were incubated for 16 hours, and on day 5 the cells were harvested and processed. All floating cells were collected. The protein (10 μg protein/well) was visualized using anti-PRK8 and anti-FX2 antibodies. The parkin variants are synthesized at the same rate, but because they are more active, they are turned-over at a higher rate, and can appear less abundant. However, after epoxomicin treatment, the protein levels are returned to the same level as wild type parkin. (FIG. 11).

### Example 4—Transduction of Rat Neuronal Cells with Construct Containing Wild Type Parkin Gene

### Stereotaxic Surgery:

Adult, female Sprague-Dawley rats were anesthetized by i.p. injection of 6 mL/kg of a 20:1 mixture of Fentanyl and Dormitor (Apoteksbolaget, Sweden). After placing the animal into a stereotaxic frame (Stoelting, Wood Dale, USA), rats were unilaterally injected with 2 μl of the rAAV5-CBA-Parkin (WT) vector in the substantia nigra according to the following coordinates: 5.2 mm posterior from bregma, 2.0 mm lateral from bregma, 7.2 mm ventral to dura while the tooth bar was adjusted to obtain a flat skull position. The injection rate of the viral vector was 0.1 μl every 15 sec and the capillary was held in place for 5 min after the injection before the needle was slowly retracted. Rats were killed 4 weeks after vector injections by an overdose of sodium pentobarbital and perfused via the ascending aorta first with 50 mL of 0.9% NaCl followed by 250 mL of ice-cold 4% paraformaldehyde for 5 min. Brains were removed and post-fixed in 4% PFA for 24 h and then transferred into 25% sucrose for cryoprotection. The brains were then cut into 35 μm thick coronal sections in six series and processed for histology.

Parkin and TH immunohistochemistry was performed on free-floating sections. Brain sections were washed with tris-buffered saline before the endogenous peroxidase activity was blocked by quenching with 3% H₂O₂ and 10% Methanol in TBS buffer for 30 min. After three washes with TBS buffer, sections were incubated in 0.05% Triton X-100 in TBS buffer (TBS-T) containing 5% of normal serum matching the species used to raise the secondary antibody for that protocol for 1 hour after which the anti-Parkin antibody [PRK8] (ab77924, 1:1000, Abcam), purified anti-Parkin antibody (5C3, 1:1000, Biolegend) or TH (Pel freeze, 1:5000) was incubated with the sections for 24 hours overnight on an orbital shaker. The next sections were rinsed with TBS-T and incubated with the corresponding biotinylated secondary antibodies (1:200, Vector Laboratories Inc, USA) in 1% BSA in TBS-T for 1 h. Sections were again washed with TBS-T and incubated with an avidin-biotin-peroxidase complex solution (Vectastain ABC kit, Vector Laboratories Inc, USA) for 1 h. Immunoreactivity was revealed with 3,3′-diaminobenzidine (DAB Safe, Saveen Werner, Sweden) and 0.01% H₂O₂. Sections were mounted on chromatin-gelatin coated glass slides, dehydrated in increasing alcohol solutions, cleared in xylene and coverslipped with DPX (06522, Sigma-Aldrich, Sweden).

### Cresyl Violet Staining:

One series of sections was processed for cresyl violet staining. Sections were mounted on chromatin-gelatin coated glass slides and dried overnight, hydrated in decreasing alcohol solutions and stained for 30 sec in pre-filtered 0.5% cresyl violet (Sigma-Aldrich, Sweden) containing 0.1% acidic acid. After a wash in H₂O, sections were dehydrated in increasing alcohol solutions, cleared in xylene and coverslipped using DPX.

### Discussion

Three different doses of the WT Parkin vector were tested in this experiment. Dose 1:1.1×10¹³ gc/mL; Dose 2:5.7×10¹³ gc/mL; and Dose 3:2.0×10¹⁴ gc/mL. Histology was performed 2.5 weeks after administration. FIG. 12 shows staining of the anterior nigra, mid nigra, and caudal nigra using Parkin PRK8 (1:2000 dilution), Parkin 5C3 (1:1000 dilution), Cresyl violet staining, and TH (Pelfreeze) (1:5000 dilution).

Dose 2 was selected as the optimal dose, based on no overt toxicity and good biodistribution and targeting of the substantia nigra.

This experiment confirmed that the AAV5 capsid injected into the ventral midbrain results in a predominantly neuronal transduction and high efficiency transduction of dopaminergic neurons in the rat. Dose 2 was selected to conduct in vivo experiments in Parkin knock-out rats.

### Example 5—Transduction of Rat Neuronal Cells with Parkin Gene Variants

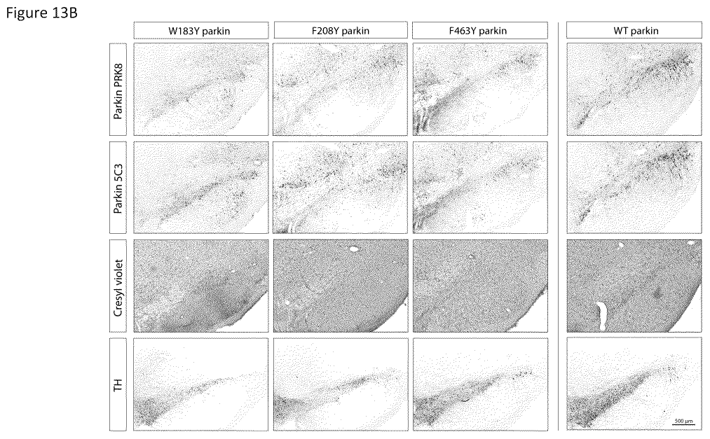

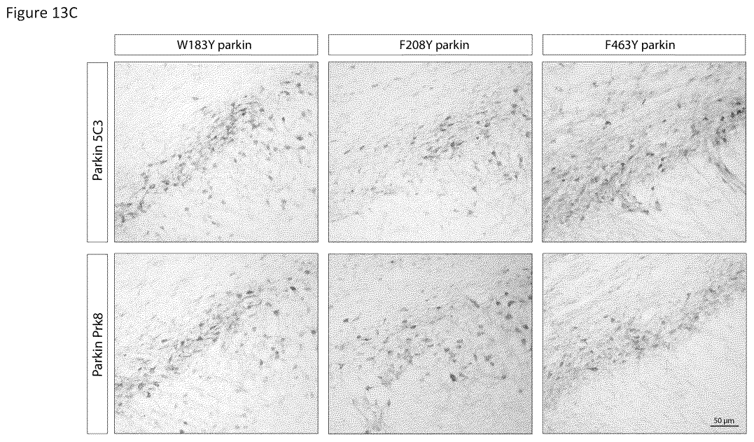

The W183Y, F208Y, and F463Y Parkin variant vectors were prepared as described in Example 3. Transduction of rat neuronal cells was carried out as in Example 4. Three different concentrations of each variant was administered-Dose 1:1.4-1.5×10¹³ gc/mL; Dose 2:6.4-7.2×10¹³ gc/mL; and Dose 3:3.19-2.1×10¹⁴ gc/mL. The vectors were administered into female SD rats (3/group; each weighing ˜200-250 g) by injecting into the unilateral SN: AP (bregma): −5.2 mm; ML (bregma): −2.0 mm; and DV (dura): −7.2 mm. Three weeks after administration, histological analysis was performed as described in Example 4 using Parkin PRK8 (1:2000 dilution), Parkin 5C3 (1:1000 dilution), Cresyl violet staining, and TH (Pelfreeze) (1:5000 dilution). FIGS. 13A-C.

### Discussion

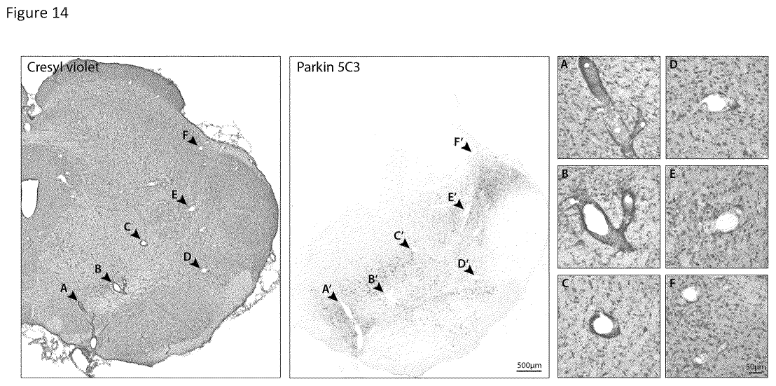

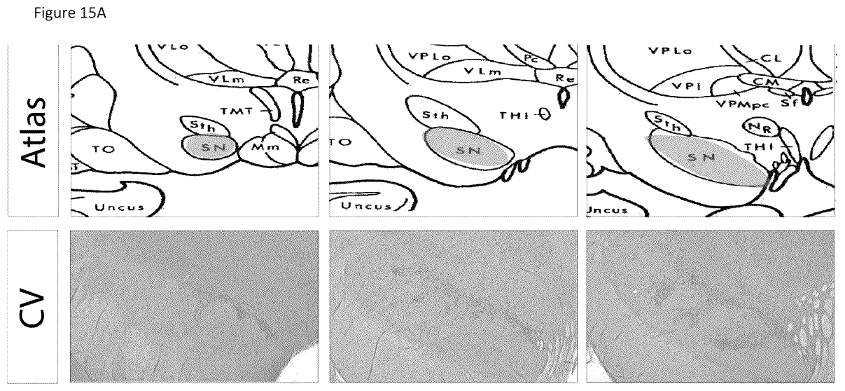

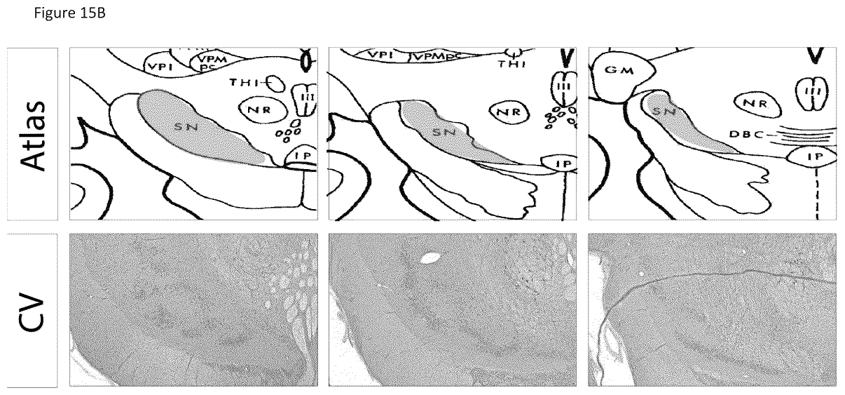

Some signs of tissue damage was observed for all mutants with different frequency and severity. FIG. 14 shows the presence of perivascular cuffing with Dose 3 of the Parkin W183Y vector.

As shown in Table 1 and Table 2, dose 2 appears to generate an immunological response and in some cases loss of TH neurons. This immunological response is not observed until administration of the highest dose of the wild type parkin, yet can be seen in lower doses of the variant parkins. Knowing the role of parkin in the immune system, the increased immunological response after administration of a variant parkin suggests that the variant parkin polypeptides are more active than the wild type polypeptide.

TABLE 1
Severity of perivascular cuffing visible with CV stain

| | | | | | WT |
|---|---|---|---|---|---|
| Dose | Case No. | W183Y | F208Y | F463Y | Parkin |
| Dose 1 | Case #1 | − | − | − | − |
| | Case #2 | − | − | − | − |
| | Case #3 | − | − | − | − |
| Dose 2 | Case #1 | − | − | − | − |
| | Case #2 | ++ | − | +++ | − |
| | Case #3 | − | ++ | − | − |
| Dose 3 | Case #1 | +++ | − | − | + |
| | Case #2 | +++ | − | ++ | + |
| | Case #3 | +++ | − | ++ | + |

Scale refers to perivascular cuffing/reaction: − not detected; + little; +++ severe

TABLE 2
Reduction/loss of TH immunoreactivity

| | | | | | WT |
|---|---|---|---|---|---|
| Dose | Case No. | W183Y | F208Y | F463Y | Parkin |
| Dose 1 | Case #1 | − | ++ | + | +/++ |
| | Case #2 | ++ | + | + | + |
| | Case #3 | ++ | + | ++ | − |
| Dose 2 | Case #1 | + | + | ++ | − |
| | Case #2 | ++ | − | ++ | − |
| | Case #3 | − | +++ | ++ | − |
| Dose 3 | Case #1 | +++ | +++ | +++ | − |
| | Case #2 | +++ | ++ | +++ | ++ |
| | Case #3 | +++ | ++ | ++ | − |

Scale refers to the extent of TH loss: − no or limited seen only at the site of injection; + mild (visible on 2 levels); ++ moderate (visible on 3 or 4 levels); +++ severe (detected on 5 or more levels)

### Example 6—Intra-Nigral Injection of Parkin Constructs in Nonhuman Primates

Four female non-human primates were included in this study. On the day of surgery, a baseline MRI scan was performed to avoid repositioning the animal. The MRI-corrected targets are shown in Table 3.

TABLE 3

| Animal | MRI-corrected targets from AC | | |
|---|---|---|---|
| ID | Antero-posterior | Lateral | Ventral |
| BO953 | −8 mm | 3.5 mm | −28.5 mm |
| BL926 | −10 mm | 3.5 mm | −27 mm |
| BV156 | −7 mm | 3.3 mm | −29 mm |
| BO803 | −8 mm | 3 mm | −27 mm |

### Animal BO953

The viral vector was produced at a titer of 7×10¹³ gc/mL, and injected at a rate of 1 μl/min into the unilateral left SNpc. A sample of the virus was used to coat the needed and syringe before loading the volume to be injected, and the remaining virus sample was kept at 4° C.

On the day of surgery (baseline), 500 μl aliquots of blood (dry tubes, serum) and 250 μl aliquots of CSF were collected as listed in Table 4. All samples were centrifuged and kept at −80° C.

TABLE 4

| Animal ID | CSF aliquots | Serum aliquots |
|---|---|---|
| BO935 | 3 | 8 |
| BL926 | 3 | 7 |
| BV156 | 3 | 8 |
| BO803 | 3 | 7 |

After surgery, the animals were observed daily for 2 weeks and weekly until euthanasia. All displayed good general appearance throughout the observation period. MRI images taken 20 days and 25 days after injection showed that the anteroposterior and lateral SN were targeted correctly in the animals. Further, there was no adverse events, such as obvious edema, hemorrhage, or inflammation, observed three of the animals (BO935, BL926, and BV156). At 25-days post injection, animal BO803 showed that, while the anteroposterior was correctly targeted, there was slight deviation targeting the lateral SN (deviation after crossing ventricle). Further, while there was no obvious edema or inflammation, hemorrhage was observed. This bleeding could have affected cell transduction by the virus.

At the end-point (euthanization) 1 blood and 1 CSF aliquots were collected. Animals were euthanized by transcardial perfusion with NaCl, extracting the brain and rinsing in ice-cold NaCl. The brain was placed in ice-cold matrix (on ice) and sliced into 4 slabs. The central slab (striatum) was sliced again, and punches were taken from the caudate and putamen bilaterally (and the rest of the sections were post-fixed). The peripheral organs (lung, heart, spleen, liver, kidney, and ovaries) were collected for biodistribution and preliminary toxicology studies. Post-mortem analysis of neutralizing antibodies was performed.

### Example 7—Injection of Construct Containing WT Parkin in Non-Human Primates

Dose 2 (1×10¹³ gc/mL) was injected into the unilateral SN in 4 non-human primates: Antero-posterior: −8 mm (from anterior commissure); Lateral: 3.5 mm (from venous sinus); Ventral: −228.5 mm (from cortex). After a survival time of 6 weeks, both biochemical and histological assays were performed on samples.

For the biochemical assays, 3 mm tissue punches from the caudate and putamen were tested for dopamine levels with HPLC, and TH levels were analyzed with Western blotting. For the histology analysis, the SN (intact; see FIG. 15A-B) and punched tissue from the caudate and putamen were visualized using Parkin PRK8 (1:2000 dilution), Parkin 5C3 (1:1000 dilution), Cresyl violet staining, and TH (Pelfreeze) (1:5000 dilution).

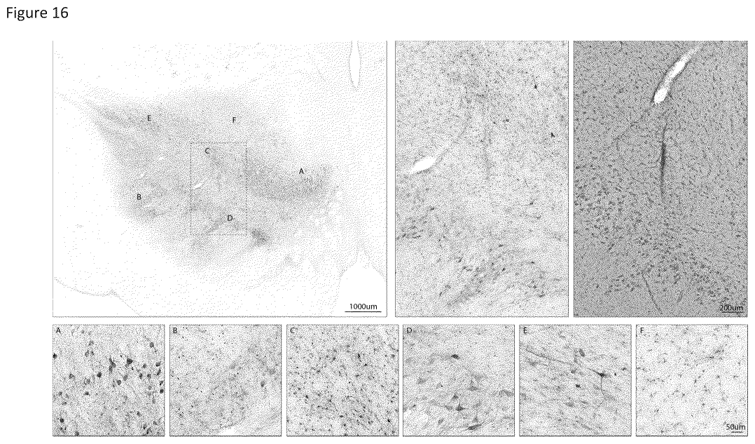

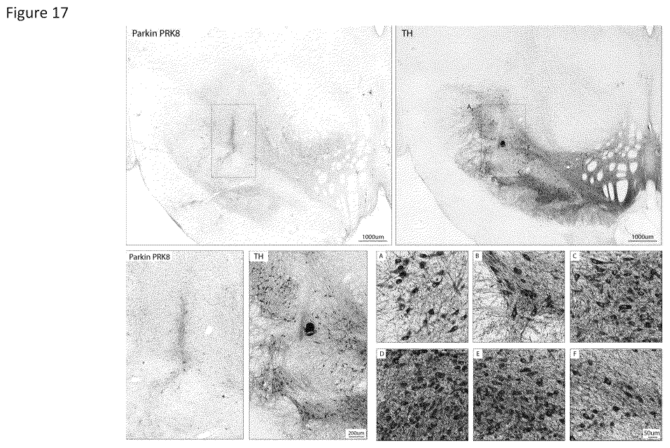

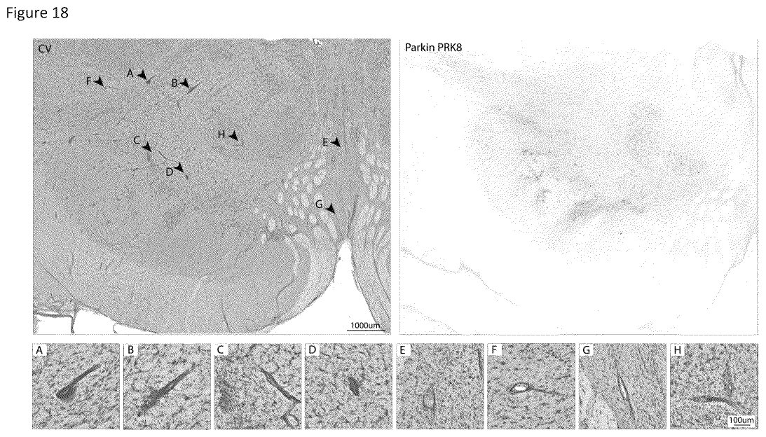

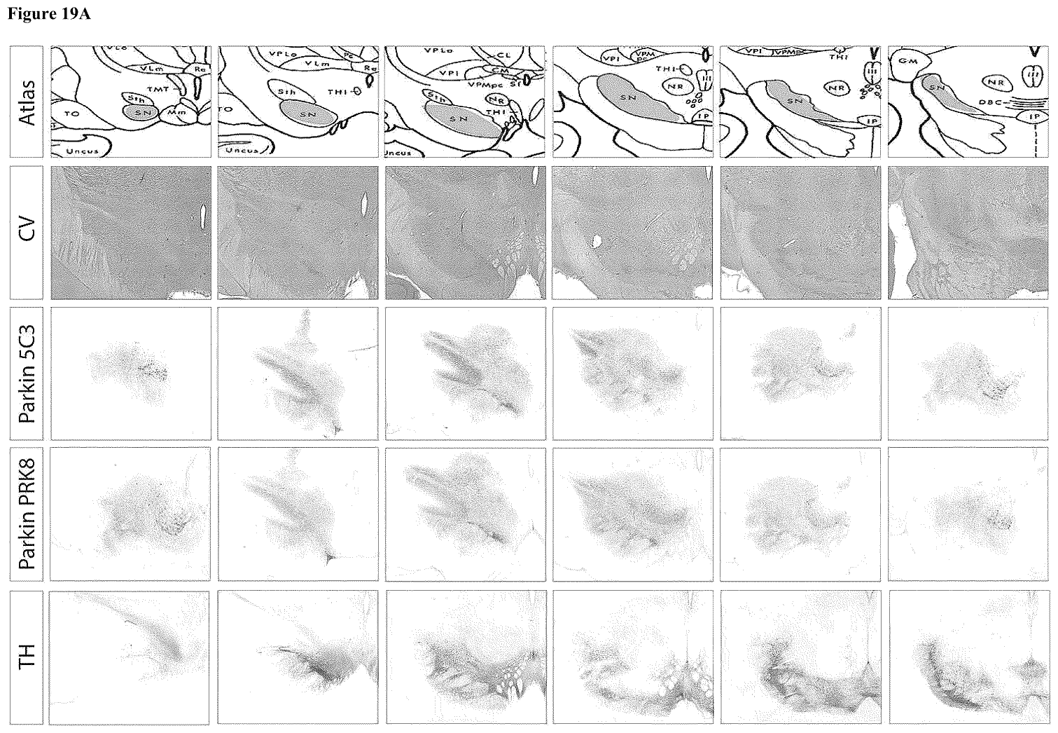

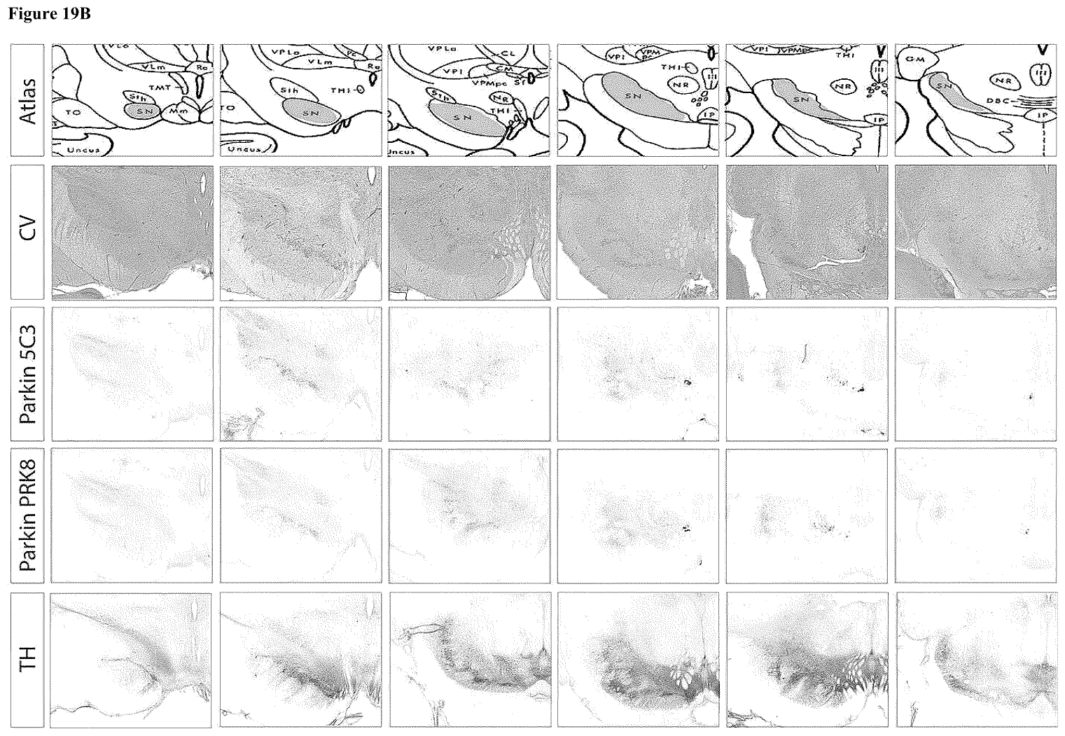

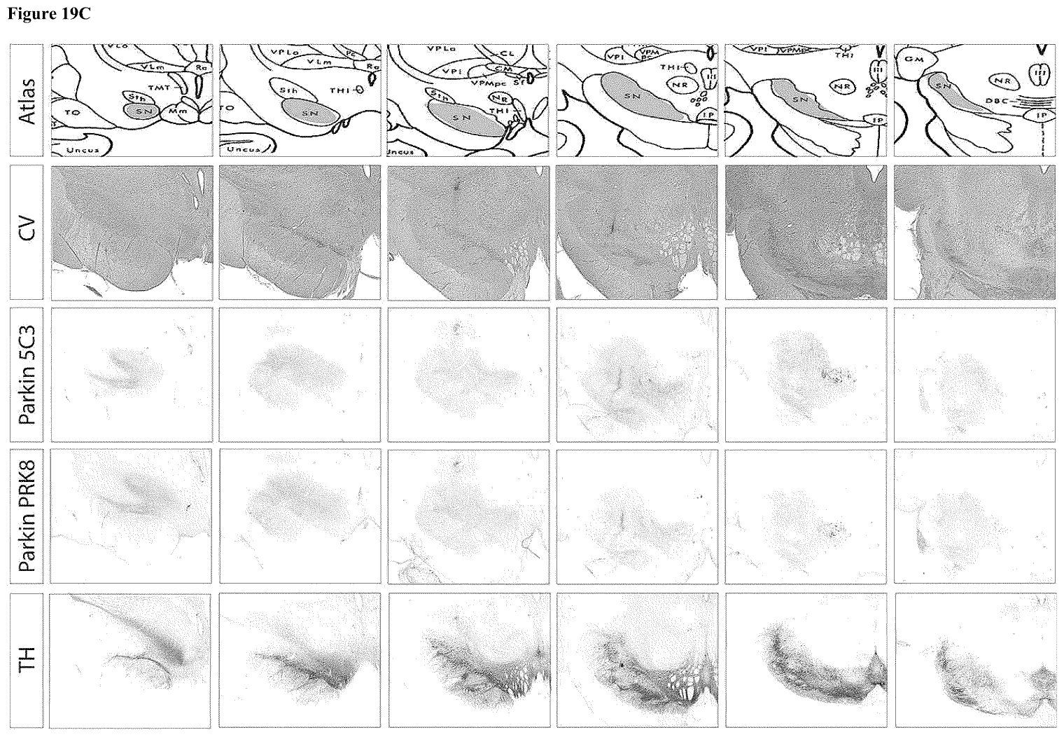

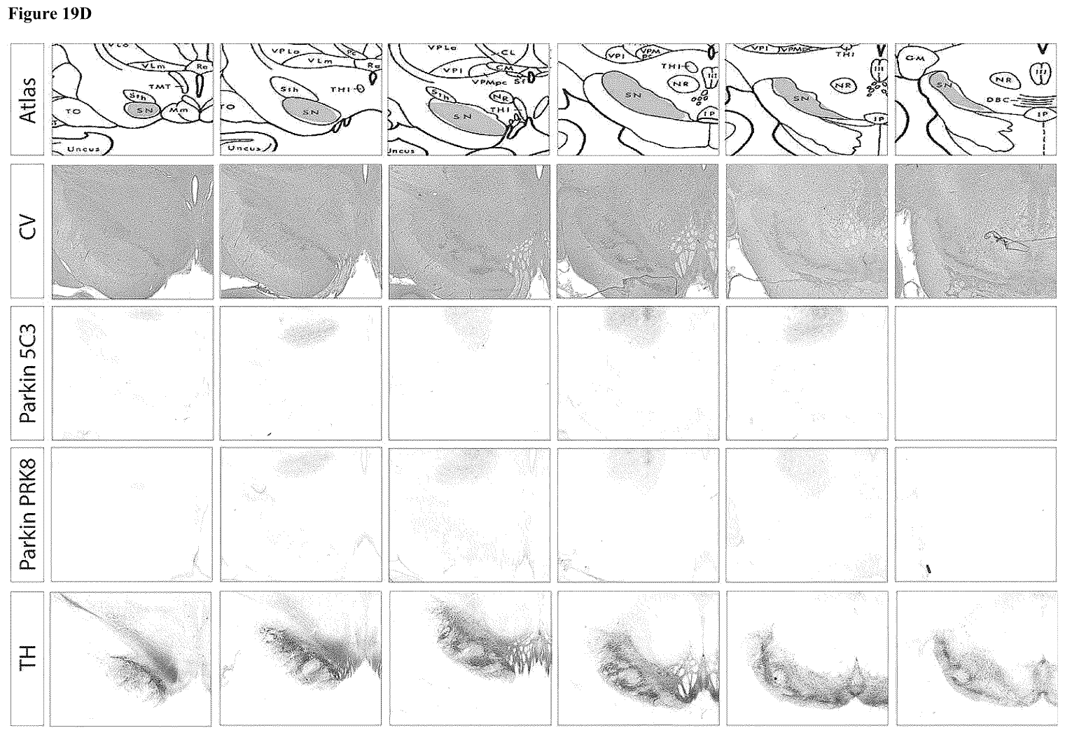

The AAV5 vector transduced both neuronal ang glial-like cells (FIG. 16), with sparse coverage of SN dopaminergic cells (FIG. 17). A tissue reaction was detected in once case (FIG. 18). The histological analysis of each of the four animals is found in FIG. 19A-D.

### INCORPORATION BY REFERENCE

All publications, patents, and patent publications cited are incorporated by reference herein in their entirety for all purposes.

### REFERENCES

Manfredsson et al. (2007) rAAV-medicated nigral human parkin over-expression partially ameliorates motor deficits via enhanced dopamine neurotransmission in a rat model of Parkinson's disease. Experimental Neurology. 207:289-301.
Niethammer et al. (2018) Gene therapy reduces Parkinson's disease symptoms by reorganizing functional brain connectivity. Sci. Transl. Med 10: eaau0713.

1. A composition comprising an AAV5 vector wherein the AAV5 vector comprises from 5′-3′:
a. an AAV2 ITR;
b. a CBA promoter;
c. a wild-type PARK2 gene comprising a polynucleotide sequence that encodes SEQ ID NO: 2;
d. a SV40 poly A tail; and
e. an AAV2 ITR;
wherein the AAV also comprises an AAV2 Rep and AAV5 cap genes.

2. The composition of claim 1, comprising a lipid or a lipid nanoparticle.

3. A pharmaceutical composition comprising the composition of claim 1, and pharmaceutically acceptable carrier.

4. A method of modifying a cell comprising administrating the pharmaceutical composition of claim 3.

5. A method of treating Parkinson's disease comprising administering the pharmaceutical composition of claim 3.

6. The method of claim 5, wherein the pharmaceutical composition is administered intrathecally.

7. The method of claim 6, wherein the administration is to the substantia nigra of a subject's brain.

8. The method of claim 7, wherein the administration results in expression of the PARK2 gene in neurons and glial cells.

9. The method of claim 8, wherein the neurons are dopaminergic neurons or oligodendrocytes.

10. The method of claim 8, wherein the glial cells are astrocytes.

11. A method of inhibiting degeneration or death of a dopaminergic neuron in a subject in need thereof, comprising administering the pharmaceutical composition of claim 3 to the brain of the subject.

12. The method of claim 11, wherein the subject is dosed with 1×10⁶-1×10¹⁴ viral genomes (vg).

13. The method of claim 12, wherein the subject is dosed with 1×10¹¹ vg.

14. The method of claim 13, wherein the dose is administered 1-30 times.
